(19) United States
(12) Patent Application Publication
(10) Pub. No.: US 2023/0382920 A1
(43) Pub. Date: Nov. 30, 2023

(54) SHMT Inhibitors

(71) Applicant: The Trustees of Princeton University, Princeton, NJ (US)

(72) Inventors: Hahn Kim, Princeton, NJ (US); Joshua D. Rabinowitz, Princeton, NJ (US)

(21) Appl. No.: 18/027,648

(22) PCT Filed: Sep. 23, 2021
(86) PCT No.: PCT/US2021/051691

§ 371 Date: Mar. 22, 2023

Related U.S. Application Data
(60) Provisional application No. 63/082,164, filed on Sep. 23, 2020.

(51) Int. Cl. C07D 491/052; A61K 45/06

(52) U.S. Cl. CPC C07D 491/052; A61K 45/06

(57) ABSTRACT

Provided herein is a compound of the following structural formula: or a pharmaceutically acceptable salt thereof, wherein values for the variables are as described herein. Also provided herein are compositions comprising a compound of structural formula (I), or a pharmaceutically acceptable salt thereof, and methods of treating a disease, disorder or condition associated with serine hydroxymethyl transferase (SHMT) activity, e.g., cancer, an autoimmune disorder, fibrosis and/or a fibrotic disease, in a subject in need thereof with a compound of structural formula (I), or a pharmaceutically acceptable salt thereof.

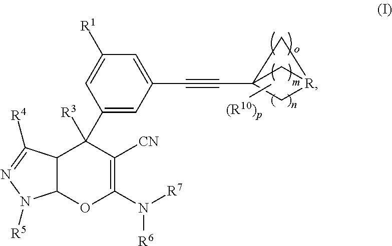

### RELATED APPLICATION

This application claims the benefit of U.S. Provisional Application No. 63/082,164, filed on Sep. 23, 2020. The entire teachings of the above application is incorporated herein by reference.

### GOVERNMENT SUPPORT

This invention was made with government support under Grant No. DK113643 awarded by the National Institutes of Health. The government has certain rights in the invention.

### BACKGROUND

U.S. Patent Application Publication Nos. US 2018/0117010 and US 2018/0072751 disclose serine hydroxymethyltransferase (SHMT) inhibitors.

Despite the discovery of SHMT inhibitors that inhibit cancer growth in in vitro growth inhibition assays and even animal models of cancer, many SHMT inhibitors lack metabolic stability and, thus, are expected to be metabolized and cleared by the body very rapidly. To overcome the lack of stability, relatively large doses of the inhibitors are needed for in vivo efficacy.

Accordingly, there is a need for SHMT inhibitors with improved metabolic stability, e.g., to be dosed on a once-daily basis and, ideally, improved potency, e.g., to decrease the dose required for in vivo efficacy.

### SUMMARY

Provided herein is a compound of the following structural formula:

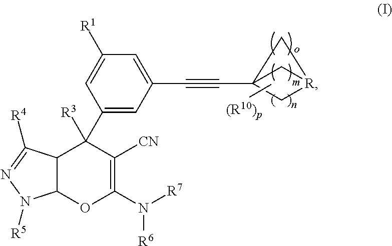

or a pharmaceutically acceptable salt thereof, wherein values for the variables (e.g., m, n, o, p, R, R¹, R³, R⁴, R⁵, R⁶, R⁷ and R¹⁰) are as described herein.

Also provided herein is a composition (e.g., pharmaceutical composition) comprising a compound described herein (e.g., a compound of any of the structural formulas depicted herein), or a pharmaceutically acceptable salt thereof, and a pharmaceutically acceptable carrier.

Also provided herein is a method of treating a disease, disorder or condition associated with serine hydroxymethyl transferase (SHMT) activity or expression in a subject in need thereof, comprising administering to the subject a therapeutically effective amount of a compound described herein (e.g., a compound of any of the structural formulas depicted herein), or a pharmaceutically acceptable salt thereof. Also provided herein is a compound for use in the treatment of a disease, disorder or condition associated with SHMT activity or expression, wherein the compound is a compound described herein (e.g., a compound of any of the structural formulas depicted herein), or a pharmaceutically acceptable salt thereof. Also provided herein is use of a compound described herein (e.g., a compound of any of the structural formulas depicted herein), or a pharmaceutically acceptable salt thereof, for the manufacture of a medicament for the treatment of a disease, disorder or condition associated with SHMT activity or expression.

Also provided herein is a method of treating a cancer in a subject in need thereof, comprising administering to the subject a therapeutically effective amount of a compound described herein (e.g., a compound of any of the structural formulas depicted herein), or a pharmaceutically acceptable salt thereof. Also provided herein is a compound for use in the treatment of a cancer, wherein the compound is a compound described herein (e.g., a compound of any of the structural formulas depicted herein), or a pharmaceutically acceptable salt thereof. Also provided herein is use of a compound described herein (e.g., a compound of any of the structural formulas depicted herein), or a pharmaceutically acceptable salt thereof, for the manufacture of a medicament for the treatment of a cancer.

Also provided herein is a method of treating an autoimmune disease in a subject in need thereof, comprising administering to the subject a therapeutically effective amount of a compound described herein (e.g., a compound of any of the structural formulas depicted herein), or a pharmaceutically acceptable salt thereof. Also provided herein is a compound for use in the treatment of an autoimmune disease, wherein the compound is a compound described herein (e.g., a compound of any of the structural formulas depicted herein), or a pharmaceutically acceptable salt thereof. Also provided herein is use of a compound described herein (e.g., a compound of any of the structural formulas depicted herein), or a pharmaceutically acceptable salt thereof, for the manufacture of a medicament for the treatment of an autoimmune disease.

Also provided herein is a method of treating fibrosis and/or a fibrotic disease in a subject in need thereof, comprising administering to the subject a therapeutically effective amount of a compound described herein (e.g., a compound of any of the structural formulas depicted herein), or a pharmaceutically acceptable salt thereof. Also provided herein is a compound for use in the treatment of fibrosis, wherein the compound is a compound described herein (e.g., a compound of any of the structural formulas depicted herein), or a pharmaceutically acceptable salt thereof. Also provided herein is use of a compound described herein (e.g., a compound of any of the structural formulas depicted herein), or a pharmaceutically acceptable salt thereof, for the manufacture of a medicament for the treatment of fibrosis.

### BRIEF DESCRIPTION OF THE DRAWINGS

The foregoing will be apparent from the following more particular description of example embodiments.

FIG. 1 is a mean plasma concentration versus time profile, and shows the mean plasma concentration of KDG-0320-0A (also referred to herein as KDG-0320) after administration of 1 mg/kg intravenous (IV), 50 mg/kg per os (PO) or 30 mg/kg intraperitoneal (IP) KDG-0320-0A to CD1 mice.

FIG. 2 is a mean plasma concentration versus time profile, and shows the mean plasma concentration of KDG-0388 after administration of 1 mg/kg IV, 200 mg/kg PO or 200 mg/kg IP KDG-0388 to CD1 mice.

FIG. 3 is a mean plasma concentration versus time profile, and shows the mean plasma concentration of KDG-0271 after administration of 1 mg/kg IV, 200 mg/kg PO or 200 mg/kg IP KDG-0271 to CD1 mice.

FIG. 4 is a mean plasma concentration versus time profile, and shows the mean plasma concentration of KDG-0271 after administration of 1 mg/kg IV, 50 mg/kg PO or 30 mg/kg IP KDG-0271 to CD1 mice.

### DETAILED DESCRIPTION

A description of example embodiments follows.

### Definitions

Compounds described herein include those described generally, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the relevant contents of which are incorporated herein by reference.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by reference herein for its chemical structure names and rules on naming chemical structures. Optionally, a name of a compound may be generated using a chemical naming program (e.g., CHEMDRAW®, version 17.0.0.206, PerkinElmer Informatics, Inc.).

“Alkyl” refers to a saturated, aliphatic, branched or straight-chain, monovalent, hydrocarbon radical having the specified number of carbon atoms. Thus, “(C₁-C₆)alkyl” means a radical having from 1-6 carbon atoms in a linear or branched arrangement. Examples of alkyl groups include methyl, ethyl, n-propyl, isopropyl, n-butyl, isobutyl, sec-butyl, t-butyl, n-pentyl, isopentyl, neopentyl, 2-methylpentyl, n-hexyl, and the like. In some embodiments, alkyl is (C₁-C₁₅)alkyl. In some embodiments, alkyl is (C₁-C₁₀)alkyl. In some embodiments, alkyl is (C₁-C₆)alkyl. In some embodiments, alkyl is (C₁-C₅)alkyl.

“Alkenyl” refers to an aliphatic, branched or straight-chain, monovalent, hydrocarbon radical having at least one carbon-carbon double bond and the specified number of carbon atoms. Thus, “(C₂-C₆)alkenyl” means a radical having at least one carbon-carbon double bond and from 2-6 carbon atoms in a linear or branched arrangement. Examples of alkenyl groups include ethenyl, 2-propenyl, 1-propenyl, 2-methyl-1-propenyl, 1-butenyl, 2-butenyl, 1-pentenyl, 2-pentenyl, 3-pentenyl, allyl, 1,3-butadienyl, 1,3-dipentenyl, 1,4-dipentenyl, 1-hexenyl, 1,3-hexenyl, 1,4-hexenyl, 1,3,5-trihexenyl, 2,4-dihexenyl, and the like. In some embodiments, alkenyl is (C₂-C₁₅)alkenyl. In some embodiments, alkenyl is (C₂-C₁₀)alkenyl. In some embodiments, alkenyl is (C₂-C₆)alkenyl. In some embodiments, alkenyl is (C₂-C₅)alkenyl.

“Alkynyl” refers to an aliphatic, branched or straight-chain, monovalent, hydrocarbon radical having at least one carbon-carbon triple bond and the specified number of carbon atoms. Thus, “(C₂-C₆)alkynyl” means a radical having at least one carbon-carbon triple bond and from 1-6 carbon atoms in a linear or branched arrangement. Examples of alkynyl groups include ethynyl, 1-propynyl, 2-propynyl, 1-butynyl, 2-butynyl, 2-methyl-1-butynyl, 1-pentynyl, 2-pentynyl, 3-pentynyl, 3-methyl-1-pentynyl, 2-methyl-1-pentynyl, 1-hexynyl, 2-hexynyl, 3-hexynyl, and the like. In some embodiments, alkynyl is (C₂-C₁₅)alkynyl. In some embodiments, alkynyl is (C₂-C₁₀)alkynyl. In some embodiments, alkynyl is (C₂-C₆)alkynyl. In some embodiments, alkynyl is (C₂-C₅)alkynyl.

“Heteroalkyl” refers to a saturated, branched or straight-chain, monovalent, hydrocarbon radical having the specified number of atoms in the chain, wherein at least one carbon atom in the chain has been replaced with a heteroatom selected from N, S and O. A heteroalkyl can contain 1, 2, 3 or 4 (e.g., 1) heteroatoms independently selected from N, S and O. When one heteroatom is S, it can be optionally mono- or di-oxygenated (i.e., —S(O)— or —S(O)₂). Thus, “(C₁-C₆)heteroalkyl” means a radical having from 1-6 carbon atoms in a linear or branched arrangement. In some embodiments, heteroalkyl is (C₁-C₁₅)heteroalkyl. In some embodiments, heteroalkyl is (C₁-C₁₀)heteroalkyl. In some embodiments, heteroalkyl is (C₁-C₆)heteroalkyl. In some embodiments, heteroalkyl is (C₁-C₅)heteroalkyl. In some embodiments, heteroalkyl is (C₁-C₃)heteroalkyl.

“Aryl” refers to a monocyclic or polycyclic (e.g., bicyclic, tricyclic), carbocyclic, aromatic ring system having the specified number of ring atoms, and includes aromatic rings fused to non-aromatic rings, as long as one of the fused rings is an aromatic hydrocarbon. Thus, “(C₆-C₁₅)aryl” means an aromatic ring system having from 6-15 ring atoms. Examples of aryl include phenyl and naphthyl. In some embodiments, aryl is (C₆-C₁₅)aryl. In some embodiments, aryl is (C₆-C₁₂)aryl. In some embodiments, aryl is (C₆-C₁₀)aryl.

“Heteroaryl” refers to a monocyclic or polycyclic (e.g., bicyclic, tricyclic), aromatic, hydrocarbon ring system having the specified number of ring atoms, wherein at least one carbon atom in the ring system has been replaced with a heteroatom selected from N, S and O. “Heteroaryl” includes heteroaromatic rings fused to non-aromatic rings, as long as one of the fused rings is a heteroaromatic hydrocarbon. Thus, “(C₅-C₁₅)heteroaryl” means a heterocyclic aromatic ring system having from 5-15 ring atoms consisting of carbon, nitrogen, sulfur and oxygen. A heteroaryl can contain 1, 2, 3 or 4 (e.g., 1 or 2) heteroatoms independently selected from N, S and O. In one embodiment, heteroaryl has 5 or 6 ring atoms (e.g., five ring atoms). Monocyclic heteroaryls include, but are not limited to, furan, oxazole, thiophene, triazole, triazene, thiadiazole, oxadiazole, imidazole, isothiazole, isoxazole, pyrazole, pyridazine, pyridine, pyrazine, pyrimidine, pyrrole, tetrazole and thiazole. Bicyclic heteroaryls include, but are not limited to, indolizine, indole, isoindole, indazole, benzimidazole, benzofuran, benzothiazole, purine, quinoline, isoquinoline, cinnoline, phthalazine, quinazoline, quinoxaline, naphthyridine and pteridine. In some embodiments, heteroaryl is (C₅-C₁₅)heteroaryl. In some embodiments, heteroaryl is (C₅-C₂)heteroaryl. In some embodiments, heteroaryl is (C₅-C₁₀)heteroaryl. In some embodiments, heteroaryl is (C₅-C₆)heteroaryl. In some embodiments, heteroaryl (e.g., (C₅-C₆)heteroaryl) is oxadiazole, such as 1,3,4-oxadiazole.

“Cycloalkyl” refers to a saturated, aliphatic, monovalent, monocyclic or polycyclic, hydrocarbon ring radical having the specified number of ring atoms. Thus, “(C₃-C₆)cycloalkyl” means a ring radical having from 3-6 ring carbons. Typically, cycloalkyl is monocyclic. Cycloalkyl includes, but is not limited to, cyclopropyl, cyclobutyl, cyclopentyl, cyclohexyl, and the like. In some embodiments, cycloalkyl is (C₃-C₁₅)cycloalkyl. In some embodiments, cycloalkyl is (C₃-C₁₂)cycloalkyl. In some embodiments, cycloalkyl is (C₃-C₈)cycloalkyl. In some embodiments, cycloalkyl is (C₃-C₆)cycloalkyl.

“Heterocyclyl” or “heterocycloalkyl” refers to a saturated, aliphatic, monocyclic or polycyclic (e.g., bicyclic, tricyclic), monovalent, hydrocarbon ring system having the specified number of ring atoms, wherein at least one carbon atom in the ring system has been replaced with a heteroatom selected from N, S and O. Thus, “(C₃-C₆)heterocyclyl” means a heterocyclic ring system having from 3-6 ring atoms. A heterocyclyl can be monocyclic, fused bicyclic, bridged bicyclic or polycyclic, but is typically monocyclic. A heterocyclyl can contain 1, 2, 3 or 4 (e.g., 1) heteroatoms independently selected from N, S and O. When one heteroatom is S, it can be optionally mono- or di-oxygenated (i.e., —S(O)— or —S(O)₂). A heterocyclyl can be saturated (i.e., contain no degree of unsaturation). Examples of monocyclic heterocyclyls include, but are not limited to, aziridine, azetidine, pyrrolidine, piperidine, piperazine, azepane, tetrahydrofuran, tetrahydropyran, morpholine, thiomorpholine, dioxide, oxirane. In some embodiments, heterocycloalkyl is (C₃-C₁₅)heterocycloalkyl. In some embodiments, heterocycloalkyl is (C₃-C₁₂)heterocycloalkyl. In some embodiments, heterocycloalkyl is (C₃-C₈)heterocycloalkyl. In some embodiments, heterocycloalkyl is (C₃-C₆)heterocycloalkyl.

“Halogen” and “halo” are used interchangeably herein and each refers to fluorine, chlorine, bromine, or iodine. In some embodiments, halogen is selected from fluoro, bromo or chloro. In some embodiments, halogen is selected from fluoro or chloro.

“Cyano” or “nitrile” means —CN.

“Hydroxy” and “hydroxyl” are used interchangeably herein and each refers to —OH.

“Hydroxyalkyl” refers to an alkyl radical wherein at least one hydrogen of the alkyl radical is replaced with hydroxy, and alkyl is as described herein. “Hydroxyalkyl” includes mono, poly, and perhydroxyalkyl groups.

“Alkoxy” refers to an alkyl radical attached through an oxygen linking atom, wherein alkyl is as described herein. Examples of alkoxy include, but are not limited to, methoxy, ethoxy, propoxy, isopropoxy, and the like.

“Haloalkyl” refers to an alkyl radical wherein at least one hydrogen of the alkyl radical is replaced with a halo, and alkyl is as described herein. Haloalkyl includes mono, poly, and perhaloalkyl groups, wherein each halogen is independently selected from fluorine, chlorine, bromine and iodine (e.g., fluorine, chlorine and bromine), and alkyl is as described herein. In one aspect, haloalkyl is perhaloalkyl (e.g., perfluoroalkyl). Haloalkyl includes, but is not limited to, fluoromethyl, difluoromethyl, trifluoromethyl and pentafluoroethyl.

“Haloalkoxy” refers to a haloalkyl radical attached through an oxygen linking atom, wherein haloalkyl is as described herein.

Groups described herein having two or more points of attachment (i.e., divalent, trivalent, or polyvalent) within the compound of the present technology are designated by use of the suffix, “ene.” For example, divalent alkyl groups are alkylene groups, divalent heteroalkyl groups are heteroalkylene, divalent aryl groups are arylene groups, divalent heteroaryl groups are heteroarylene groups, and so forth. Substituted groups having a single point of attachment to the compound of the present technology are not referred to using the “ene” designation. Thus, e.g., trifluoromethyl is not referred to herein as trifluoromethylene.

It is understood that substituents on the compounds of the invention can be selected by one of ordinary skill in the art to provide compounds that are chemically stable and that can be readily synthesized by techniques known in the art, as well as those methods set forth below. The term “stable,” as used herein, refers to compounds that are not substantially altered when subjected to conditions to allow for their production, detection and, in certain embodiments, recovery, purification and use for one or more of the purposes disclosed herein.

Combinations of substituents envisioned by this invention are preferably those that result in the formation of stable or chemically feasible compounds.

A designated group is unsubstituted, unless otherwise indicated, e.g., by provision of a variable that denotes allowable substituents for a designated group. For example, R¹⁰ in Structural Formula I denotes allowable substituents for the ring system to which R¹⁰ is attached. However, when the term “substituted” precedes a designated group, it means that one or more hydrogens of the designated group are replaced with a suitable substituent. Unless otherwise indicated, an “optionally substituted” group or “substituted or unsubstituted” group can have a suitable substituent at each substitutable position of the group and, when more than one position in any given structure may be substituted with more than one substituent selected from a specified group, the substituent can be the same or different at every position. Alternatively, an “optionally substituted” group or “substituted or unsubstituted” group can be unsubstituted. An “optionally substituted” group is, in some embodiments, substituted with 0-5 (e.g., 0-5, 0-3, 0, 1, 2, 3, 4, 5) substituents.

Suitable substituents for a substituted or optionally substituted group include, but are not limited to, for example, halogen, hydroxyl, carbonyl (such as carboxyl, alkoxycarbonyl, formyl, or acyl), thiocarbonyl (such as thioester, thioacetate, or thioformate), alkyl, alkoxy, alkylthio, acyloxy, phosphoryl, phosphate, phosphonate, amino, amido, amidino, imino, cyano, nitro, azido, sulfhydryl, alkylthio, sulfate, sulfonate, sulfamoyl, sulfonamido, sulfonyl, cycloalkyl, heterocyclyl, aryl or heteroaryl. It will be understood by those skilled in the art that substituents can themselves be substituted, if appropriate and where indicated. For instance, substituent(s) of a substituted alkyl may include substituted and unsubstituted forms of hydroxyl, amino, imino, amido, phosphoryl (including phosphonate and phosphinate), sulfonyl (including sulfate, sulfonamido, sulfamoyl and sulfonate) and carbonyls (including ketones, aldehydes, carboxylates, and esters), and the like.

In some embodiments, a substituted group or optionally substituted group is substituted or optionally substituted, respectively, with one or more (e.g., one, two, three, four or five) substituents independently selected from halo, hydroxy, cyano, azido, nitro, —N(R¹⁴)C(O)N(R¹⁴)(R¹⁵), —OC(O)N(R¹⁴)(R¹⁵), —N(R¹⁴)C(O)O(R¹⁵), —N(R¹⁴)(R¹⁵), —C(O)N(R¹⁴)(R¹⁵), —N(R¹⁴)C(O)(R¹⁵), —C(O)OR¹⁴, —OC(O)R¹⁴, alkyl, alkenyl, alkynyl, haloalkyl, hydroxyalkyl, cycloalkyl, heterocycloalkyl, alkoxy, haloalkoxy, aryl and heteroaryl, wherein R¹⁴ and R¹⁵ are each independently selected from hydrogen or optionally substituted (e.g., unsubstituted) alkyl, alkenyl, alkynyl, haloalkyl, hydroxyalkyl, cycloalkyl, heterocycloalkyl, aryl or heteroaryl (e.g., alkyl). In some embodiments, a substituted group or optionally substituted group is substituted or optionally substituted, respectively, with one or more (e.g., one, two, three, four or five) substituents independently selected from halo, hydroxy, cyano, azido, nitro, —N(R¹⁴)C(O)N(R¹⁴)(R¹⁵), —OC(O)N(R¹⁴)(R¹⁵), —N(R¹⁴)C(O)O(R¹⁵), —N(R¹⁴)(R¹⁵), —C(O)N(R¹⁴)(R¹⁵), —N(R¹⁴)C(O)(R¹⁵), —C(O)OR¹⁴, —OC(O)R¹⁴, alkyl, alkenyl, alkynyl, haloalkyl, hydroxyalkyl, cycloalkyl, heterocycloalkyl, alkoxy, haloalkoxy, aryl and heteroaryl, wherein R¹⁴ and R¹⁵ are each independently selected from hydrogen or optionally substituted (e.g., unsubstituted) alkyl, alkenyl, alkynyl, haloalkyl, hydroxyalkyl, cycloalkyl, heterocycloalkyl, aryl or heteroaryl (e.g., alkyl). In some embodiments, substituents are independently selected from halo, hydroxy, cyano, alkyl, haloalkyl, hydroxyalkyl, alkoxy or haloalkoxy. In some embodiments, substituents are independently selected from halo, hydroxy, cyano, alkyl, haloalkyl or hydroxyalkyl.

As used herein, the term “pharmaceutically acceptable” refers to species which are, within the scope of sound medical judgment, suitable for use in contact with the tissues of mammals without undue toxicity, irritation, allergic response and the like, and are commensurate with a reasonable benefit/risk ratio. For example, a substance is pharmaceutically acceptable when it is suitable for use in contact with cells, tissues or organs of animals or humans without excessive toxicity, irritation, allergic response, immunogenicity or other adverse reactions, in the amount used in the dosage form according to the dosing schedule, and commensurate with a reasonable benefit/risk ratio.

As used herein, the term “pharmaceutically acceptable salt” refers to those salts which are, within the scope of sound medical judgment, suitable for use in contact with the tissues of mammals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the compounds described herein include salts derived from suitable inorganic and organic acids, and suitable inorganic and organic bases.

Examples of pharmaceutically acceptable acid addition salts are salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable acid addition salts include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N⁺((C₁-C₄)alkyl)₄ salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

Compounds described herein can also exist as various “solvates” or “hydrates.” A “hydrate” is a compound that exists in a composition with one or more water molecules. The composition can include water in stoichiometic quantities, such as a monohydrate or a dihydrate, or can include water in random amounts. A “solvate” is similar to a hydrate, except that a solvent other than water, such as methanol, ethanol, dimethylformamide, diethyl ether, or the like replaces water. Mixtures of such solvates or hydrates can also be prepared. The source of such solvate or hydrate can be from the solvent of crystallization, inherent in the solvent of preparation or crystallization, or adventitious to such solvent.

Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention. In all provided structures, any hydrogen atom can also be independently selected from deuterium (²H), tritium (³H) and/or fluorine (F). Such compounds are useful, for example, as analytical tools, as probes in biological assays, or as therapeutic agents in accordance with the present invention.

Compounds disclosed herein may exist as stereoisomers. For example, compounds disclosed herein may have asymmetric centers, chiral axes, and chiral planes (e.g., as described in. E. L. Eliel and S. H. Wilen, Stereo-chemistry of Carbon Compounds, John Wiley & Sons, New York, 1994, pages 1119-1190), and occur as racemates, racemic mixtures, or as individual diastereomers or enantiomers. For example, it will be appreciated that certain compounds of structural formula (I) (e.g., compounds of structural formula (I) wherein R⁵ is hydrogen) may exist as tautomeric forms represented, for example, by the following chemical equation:

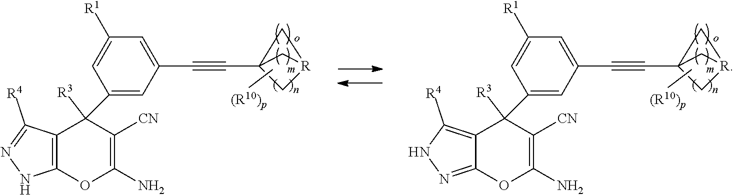

Unless otherwise indicated, all possible isomers and mixtures thereof, including optical isomers, rotamers, tautomers and cis- and trans-isomers, are included in the present invention.

When a disclosed compound is depicted by structure without indicating the stereochemistry, and the compound has one chiral center, it is to be understood that the structure encompasses one enantiomer or diastereomer of the compound separated or substantially separated from the corresponding optical isomer(s), a racemic mixture of the compound and mixtures enriched in one enantiomer or diastereomer relative to its corresponding optical isomer(s).

When a disclosed compound is depicted by a structure indicating stereochemistry, and the compound has more than one chiral center, the stereochemistry indicates relative stereochemistry, rather than the absolute configuration of the substituents around the one or more chiral carbon atoms. “R” and “S” are used to indicate the absolute configuration of substituents around one or more chiral carbon atoms.

“Enantiomers” are pairs of stereoisomers that are non-superimposable mirror images of one another, most commonly because they contain an asymmetrically substituted carbon atom that acts as a chiral center.

“Diastereomers” are stereoisomers that are not related as mirror images, most commonly because they contain two or more asymmetrically substituted carbon atoms.

“Racemate” or “racemic mixture,” as used herein, refer to a mixture containing equimolar quantities of two enantiomers of a compound. Such mixtures exhibit no optical activity (i.e., they do not rotate a plane of polarized light).

Percent enantiomeric excess (ee) is defined as the absolute difference between the mole fraction of each enantiomer multiplied by 100% and can be represented by the following equation:

$$ee = \vert \frac{R - S}{R + S} \vert \times 100 \% ,$$

where R and S represent the respective fractions of each enantiomer in a mixture, such that R+S=1. An enantiomer may be present in an ee of at least or about 50%, about 60%, about 70%, about 80%, about 90%, about 95%, about 96%, about 97%, about 98%, about 99% or about 99.9%.

Percent diastereomeric excess (de) is defined as the absolute difference between the mole fraction of each diastereomer multiplied by 100% and can be represented by the following equation:

$$de = \vert \frac{D 1 - ( D 2 + D 3 + D 4 … )}{D 1 + ( D 2 + D 3 + D 4 … )} \vert \times 100 \% ,$$

where D1 and (D2+D3+D4 . . . ) represent the respective fractions of each diastereomer in a mixture, such that D1+(D2+D3+D4 . . . )=1. A diastereomer may be present in a de of at least or about 50%, about 60%, about 70%, about 80%, about 90%, about 95%, about 96%, about 97%, about 98%, about 99% or about 99.9%.

Methods of obtaining an optical isomer separated or substantially separated from the corresponding optical isomer(s) are known in the art. For example, an optical isomer can be purified from a racemic mixture by well-known chiral separation techniques, such as, but not limited to, normal- and reverse-phase chromatography, and crystallization. An optical isomer can also be prepared by the use of chiral intermediates or catalysts in synthesis. In some cases, compounds having at least some degree of enantiomeric enrichment can be obtained by physical processes, such as selective crystallization of salts or complexes formed with chiral adjuvants.

As used herein, the term “compound of the disclosure” refers to a compound of any structural formula depicted herein (e.g., a compound of structural formula I or a subformula thereof)), as well as isomers, such as stereoisomers (including diastereoisomers, enantiomers and racemates) and tautomers thereof, isotopologues thereof, and inherently formed moieties (e.g., polymorphs and/or solvates, such as hydrates) thereof. When a moiety is present that is capable of forming a salt, then salts are included as well, in particular, pharmaceutically acceptable salts.

“Pharmaceutically acceptable carrier” refers to a carrier or excipient that does not destroy the pharmacological activity of the agent with which it is formulated and is, within the scope of sound medical judgment, suitable for use in contact with the tissues of mammals without undue toxicity, irritation, allergic response and the like, and is commensurate with a reasonable benefit/risk ratio. Pharmaceutically acceptable carriers that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

“Treating,” as used herein, refers to taking steps to deliver a therapy to a subject, such as a mammal, in need thereof (e.g., as by administering to a mammal one or more therapeutic agents). “Treating” includes inhibiting the disease or condition (e.g., as by slowing or stopping its progression or causing regression of the disease or condition), and relieving the symptoms resulting from the disease or condition.

“Administering,” as used herein, refers to taking steps to deliver an agent to a subject, such as a mammal, in need thereof (e.g., as by administering to a mammal one or more therapeutic agents). Administering can be performed, for example, once, a plurality of times, and/or over one or more extended periods. Administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

“A therapeutically effective amount” is an amount effective, at dosages and for periods of time necessary, to achieve a desired therapeutic result (e.g., treatment, healing, inhibition or amelioration of physiological response or condition, etc.). The full therapeutic effect does not necessarily occur by administration of one dose, and may occur only after administration of a series of doses. Thus, a therapeutically effective amount may be administered in one or more administrations. A therapeutically effective amount may vary according to factors such as disease state, age, sex, and weight of a mammal, mode of administration and the ability of a therapeutic, or combination of therapeutics, to elicit a desired response in an individual. A therapeutically effective amount of an agent to be administered can be determined by a clinician of ordinary skill using the guidance provided herein and other methods known in the art.

As used herein, “subject” includes humans, domestic animals, such as laboratory animals (e.g., dogs, monkeys, pigs, rats, mice, etc.), household pets (e.g., cats, dogs, rabbits, etc.) and livestock (e.g., pigs, cattle, sheep, goats, horses, etc.), and non-domestic animals. In some embodiments, a subject is a mammal (e.g., a non-human mammal). In some embodiments, a subject is a human.

When introducing elements disclosed herein, the articles “a,” “an,” “the,” and “said” are intended to mean that there are one or more of the elements.

The terms “comprising,” “having” and “including” are intended to be open-ended and mean that there may be additional elements other than the listed elements.

### Compounds

A first embodiment provides a compound of the following structural formula:

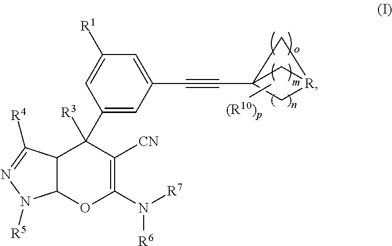

or a pharmaceutically acceptable salt thereof, wherein:
n is 0, 1, 2 or 3;
n is 1, 2 or 3;
is 0, 1, 2 or 3, wherein when o is 0, the ring containing R is monocyclic;
p is 0, 1, 2, 3, 4 or 5;
R is —C(H)(N(R¹)C(O)R²)—, —C(H)(N(R⁸)CH₂C(O)R²)—, —N(C(O)R²)—, —N(CH₂C(O)R²)—, —C(H)(hydroxy(C₁-C₁₀)alkyl)-, —N(hydroxy(C₁-C₁₀)alkyl)-, —C(H)((C₅-C₆)heteroaryl)-, —C(H)(N(R⁸)(C₅-C₆)heteroaryl)-, —C(H)((C₃-C₇)heterocyclyl)- or —N((C₅-C₆)heteroaryl)- when o is 0, and —C(N(R¹)C(O)R²)—, —C(N(R¹)CH₂C(O)R²)—, —C(hydroxy(C₁-C₁₀)alkyl)-, —C((C₅-C₆)heteroaryl)-, —C(N(R′)(C₅-C₆)heteroaryl)- or —C((C₃-C₂)heterocyclyl)- when o is other than 0, wherein the heteroaryl can be substituted with one or more R¹¹, and the heterocyclyl can be substituted with one or more substituents selected from oxo or R¹¹;
R¹ is —H, halogen, hydroxyl, nitro, cyano, —SOR²¹, —S(O)₂R²¹, —S(O)₂NR²⁰R²², —OR²¹, —OC(O)R²², —C(O)OR²², —C(O)R²¹, —C(O)NR²⁰R²², —NR²⁰R²², —N(R²²)C(O)R²¹, —NS(O)₂R²², substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted haloalkyl, or substituted or unsubstituted haloalkoxy;
R² is substituted or unsubstituted alkoxy, substituted or unsubstituted cycloalkoxy, substituted or unsubstituted arylalkoxy, substituted or unsubstituted heteroarylalkoxy, substituted or unsubstituted alkyl, —NR^{a}R^{b} or —CH₂NR^{a}R^{b}, wherein the cycloalkoxy can be substituted with one or more R¹²
   R^{a} is hydrogen or substituted or unsubstituted alkyl;
   R^{b} is substituted or unsubstituted alkyl;
R³ is —H, halogen, hydroxyl, nitro, nitrile, —SOR²¹, —S(O)₂R²¹, —S(O)₂NR²⁰R²², —OR²¹, —OC(O)R²², —C(O)OR²², —C(O)R²¹, —C(O)NR²⁰R²², —NR²⁰R²², —N(R²²)C(O)R²¹, —NS(O)₂R²², substituted or unsubstituted alkyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted haloalkyl, or substituted or unsubstituted haloalkoxy;
R⁴ is —H, substituted or unsubstituted alkyl, substituted or unsubstituted cycloalkyl, substituted or unsubstituted aryl, substituted or unsubstituted heteroaryl, substituted or unsubstituted arylalkyl, or substituted or unsubstituted heteroarylalkyl;
R⁵ is —H, —C(O)R²¹, substituted or unsubstituted alkyl, substituted or unsubstituted cycloalkyl, substituted or unsubstituted aryl, substituted or unsubstituted heteroaryl, substituted or unsubstituted arylalkyl, or substituted or unsubstituted heteroarylalkyl;
R⁶ and R⁷ are each independently —H, —C(O)R²¹, substituted or unsubstituted alkyl, substituted or unsubstituted cycloalkyl, substituted or unsubstituted aryl, substituted or unsubstituted heteroaryl, substituted or unsubstituted arylalkyl, or substituted or unsubstituted heteroarylalkyl; or R⁶ and R⁷ taken together with the nitrogen atom to which they are attached form a substituted or unsubstituted 3-6 membered ring;
each R⁸ is independently hydrogen or substituted or unsubstituted alkyl; and
R¹⁰, R¹¹ and R¹², for each occurrence, are independently halo, hydroxy, cyano or substituted or unsubstituted alkyl;
each occurrence of R²¹ is independently selected from substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted aryl, or substituted or unsubstituted heteroaryl; and
each occurrence of R²⁰ and R²² is independently selected from —H, substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted aryl, or substituted or unsubstituted heteroaryl.

In a first aspect of the first embodiment:
n is 0, 1 or 2;
m is 1 or 2;
p is 0, 1, 2, 3, 4 or 5;
o is 0, 1, 2 or 3, wherein when o is 0, the ring containing R is monocyclic;
R is —C(H)(N(R⁸)C(O)R²)—, —C(H)(N(R⁸)CH₂C(O)R²)—, —N(C(O)R²)—, —N(CH₂C(O)R²)—, —C(H)(hydroxy(C₁-C₁₀)alkyl)-, —N(hydroxy(C₁-C₁₀)alkyl)-, —C(H)((C₅-C₆)heteroaryl)-, —C(H)(N(R⁸)(C₅-C₆)heteroaryl)-, —C(H)((C₃-C₇)heterocyclyl)- or —N((C₅-C₆)heteroaryl)- when o is 0, and —C(N(R⁸)C(O)R²)—, —C(N(R⁸)CH₂C(O)R²)—, —C(hydroxy(C₁-C₁₀)alkyl)-, —C((C₅-C₆)heteroaryl)-, —C(N(R⁸)(C₅-C₆)heteroaryl)- or —C((C₃-C₇)heterocyclyl)- when o is other than 0, wherein the heteroaryl can be substituted with one or more R¹¹, and the heterocyclyl can be substituted with one or more substituents selected from oxo or R¹¹;
R¹ is (C₁-C₆)alkyl, hydroxy(C₁-C₆)alkyl, halo(C₁-C₆)alkyl, (C₃-C₆)cycloalkyl, halo or cyano;
R² is (C₁-C₁₀)alkoxy, (C₁-C₁₀)alkoxy(C₁-C₁₀)alkoxy, hydroxy(C₁-C₁₀)alkoxy, halo(C₁-C₁₀)alkoxy, (C₃-C₆)cycloalkoxy, (C₆-C₁₂)aryl(C₁-C₁₀)alkoxy, (C₅-C₁₂)heteroaryl(C₁-C₁₀)alkoxy, (C₁-C₁₀)alkyl, —NR^{a}R^{b} or —CH₂NR^{a}R^{b}, wherein the cycloalkoxy can be substituted with one or more R¹²
   R^{a} is hydrogen or (C₁-C₁₀)alkyl;
   R^{b} is (C₁-C₁₀)alkyl;
R³ is (C₁-C₆)alkyl or (C₁-C₆)cycloalkyl;
R⁴ is (C₁-C₆)alkyl, (C₁-C₆)haloalkyl, hydroxy(C₁-C₆)alkyl or (C₃-C₆)cycloalkyl;
R⁵, R⁶ and R⁷ are each hydrogen;
each R⁸ is independently hydrogen or (C₁-C₁₀)alkyl; and
R¹⁰, R¹¹ and R¹², for each occurrence, are independently halo, hydroxy, cyano, (C₁-C₆)alkyl, halo(C₁-C₆)alkyl, hydroxy(C₁-C₆)alkyl.
   Values for the remaining variables are as described in the first embodiment.

In a second aspect of the first embodiment, n is 0 or 1 (e.g., 1). Values for the remaining variables are as described in the first embodiment, or the first aspect thereof.

In a third aspect of the first embodiment, m is 1. Values for the remaining variables are as described in the first embodiment, or first or second aspect thereof.

In a fourth aspect of the first embodiment, o is 0 and the ring containing R is monocyclic. Stated otherwise, when o is 0, the ring containing R has the following structural formula:

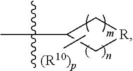

wherein values for the variables (e.g., m, n, p, R, R¹⁰) are as described herein (e.g., in the first embodiment, or any aspect thereof). By way of contrast, when n is 0, the ring containing R may be bicyclic (as, for example, when o is other than 0) or monocyclic (as, for example, when o is 0) because there remains a covalent bond between R and the carbon connecting the ring containing R to the remainder of the compound. Thus, for example, when o is 0 and n is 0, the ring containing R has the following structural formula:

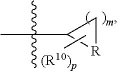

wherein values for the variables (e.g., m, R, R¹⁰) are as described herein (e.g., in the first embodiment, or any aspect thereof). Values for the remaining variables are as described in the first embodiment, or first through third aspects thereof.

In a fifth aspect of the first embodiment, o is 1, 2 or 3 (e.g., 1). Values for the remaining variables are as described in the first embodiment, or first through fourth aspects thereof.

In a sixth aspect of the first embodiment, p is 0 or 1 (e.g., 0). Values for the remaining variables are as described in the first embodiment, or first through fifth aspects thereof.

In a seventh aspect of the first embodiment, R¹ is —CH₂OH, —C(CH₃)₂OH, —CH₂F, —CF₃ or —F. Values for the remaining variables are as described in the first embodiment, or first through sixth aspects thereof.

In an eighth aspect of the first embodiment, R¹ is —CH₂OH. Values for the remaining variables are as described in the first embodiment, or first through seventh aspects thereof.

In a ninth aspect of the first embodiment, R² is (C₁-C₁₀)alkoxy (e.g., tert-butoxy). Values for the remaining variables are as described in the first embodiment, or first through eighth aspects thereof.

In a tenth aspect of the first embodiment, R^{a} is hydrogen. Values for the remaining variables are as described in the first embodiment, or first through ninth aspects thereof.

In an eleventh aspect of the first embodiment, R^{b} is tert-butyl. Values for the remaining variables are as described in the first embodiment, or first through tenth aspects thereof.

In a twelfth aspect of the first embodiment, R³ is isopropyl, n-propyl, ethyl, methyl, cyclopropyl or cyclobutyl (e.g., isopropyl). Values for the remaining variables are as described in the first embodiment, or first through eleventh aspects thereof.

In a thirteenth aspect of the first embodiment, R⁴ is methyl. Values for the remaining variables are as described in the first embodiment, or first through twelfth aspects thereof.

In a fourteenth aspect of the first embodiment, R⁸ is hydrogen. Values for the remaining variables are as described in the first embodiment, or first through thirteenth aspects thereof.

In a fifteenth aspect of the first embodiment, R⁵, R⁶ and R⁷ are each hydrogen. Values for the remaining variables are as described in the first embodiment, or first through fourteenth aspects thereof.

In a sixteenth aspect of the first embodiment, R³ is (C₁-C₆)alkyl or (C₁-C₆)cycloalkyl. Values for the remaining variables are as described in the first embodiment, or first through fifteenth aspects thereof.

In a seventeenth aspect of the first embodiment, R⁴ is (C₁-C₆)alkyl, (C₁-C₆)haloalkyl, hydroxy(C₁-C₆)alkyl or (C₃-C₆)cycloalkyl. Values for the remaining variables are as described in the first embodiment, or first through sixteenth aspects thereof.

In an eighteenth aspect of the first embodiment, R is —C(H)(N(R⁸)C(O)R²)— or —N(C(O)R²)—, when o is 0, and —C(N(R⁸)C(O)R²)—, when o is other than 0. Values for the remaining variables are as described in the first embodiment, or first through seventeenth aspects thereof.

In a nineteenth aspect of the first embodiment, m is 2. Values for the remaining variables are as described in the first embodiment, or first through eighteenth aspects thereof.

In a twentieth aspect of the first embodiment, n is 1 or 2. Values for the remaining variables are as described in the first embodiment, or first through nineteenth aspects thereof.

In a twenty-first aspect of the first embodiment, o is 0 or 1. Values for the remaining variables are as described in the first embodiment, or first through twentieth aspects thereof.

In a twenty-second aspect of the first embodiment, m is 1, n is 1, and o is 0. Values for the remaining variables are as described in the first embodiment, or first through twenty-first aspects thereof.

In a twenty-third aspect of the first embodiment, m is 2, n is 2, and o is 0. Values for the remaining variables are as described in the first embodiment, or first through twenty-second aspects thereof.

In a twenty-fourth aspect of the first embodiment, m is 1, n is 1, and o is 1. Values for the remaining variables are as described in the first embodiment, or first through twenty-third aspects thereof.

In a twenty-fifth aspect of the first embodiment, R is —C(H)(N(R⁸)C(O)R²)— or —N(C(O)R²)— when o is 0, and —C(N(R⁸)C(O)R²)— when o is other than 0. Values for the remaining variables are as described in the first embodiment, or first through twenty-fourth aspects thereof.

In a twenty-sixth aspect of the first embodiment, R is —C(H)((C₅-C₆)heteroaryl)-, —C(H)(N(R′)(C₅-C₆)heteroaryl)- or —N((C₅-C₆)heteroaryl)- when o is 0, and —C((C₅-C₆)heteroaryl)- or —C(N(R⁸)(C₅-C₆)heteroaryl)- when o is other than 0, wherein the heteroaryl can be substituted with one or more R¹¹. Values for the remaining variables are as described in the first embodiment, or first through twenty-fifth aspects thereof.

In a twenty-seventh aspect of the first embodiment, R¹⁰, for each occurrence, is independently halo (e.g., fluoro), (C₁-C₆)alkyl (e.g., methyl) or halo(C₁-C₆)alkyl (e.g., trifluoromethyl). Values for the remaining variables are as described in the first embodiment, or first through twenty-sixth aspects thereof.

In a twenty-eighth aspect of the first embodiment, R¹⁰, for each occurrence, is independently fluoro, methyl or trifluoromethyl. Values for the remaining variables are as described in the first embodiment, or first through twenty-seventh aspects thereof.

In a twenty-ninth aspect of the first embodiment, R¹¹, for each occurrence, is independently (C₁-C₆)alkyl (e.g., methyl, isopropyl). Values for the remaining variables are as described in the first embodiment, or first through twenty-eighth aspects thereof.

In a thirtieth aspect of the first embodiment, R¹², for each occurrence, is independently halo (e.g., fluoro), (C₁-C₆)alkyl (e.g., methyl) or halo(C₁-C₆)alkyl (e.g., trifluoromethyl). Values for the remaining variables are as described in the first embodiment, or first through twenty-ninth aspects thereof.

In a thirty-first aspect of the first embodiment, R¹ is (C₁-C₆)alkyl, hydroxy(C₁-C₆)alkyl, halo(C₁-C₆)alkyl, —(C₁-C₆)alkyl-OC(O)—(C₁-C₆)alkyl, —(C₁-C₆)alkyl-OC(O)—C(H)(NH₂)—(C₁-C₆)alkyl, (C₃-C₆)cycloalkyl, halo or cyano. Values for the remaining variables are as described in the first embodiment, or first through thirtieth aspects thereof.

In a thirty-second aspect of the first embodiment, the compound has the following structural formula:

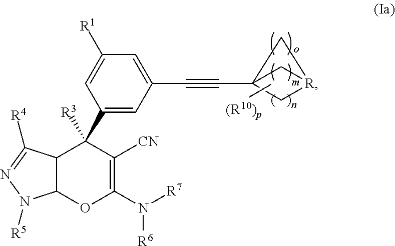

or a pharmaceutically acceptable salt thereof, wherein values for the variables (e.g., m, n, o, p, R, R¹, R³, R⁴. R¹, R⁶, R⁷, R¹⁰) are as described in the first embodiment, or the first through thirty-first aspects thereof.

In a thirty-third aspect of the first embodiment, the compound has the following structural formula:

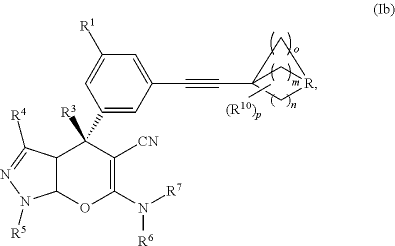

or a pharmaceutically acceptable salt thereof, wherein values for the variables (e.g., m, n, o, p, R, R¹, R³, R⁴. R⁵, R⁶, R⁷, R¹⁰) are as described in the first embodiment, or the first through thirty-first aspects thereof.

A second embodiment provides a compound of the following structural formula:

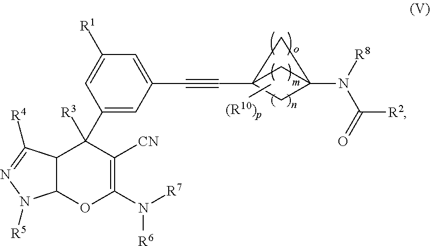

or a pharmaceutically acceptable salt thereof, wherein values for the variables (e.g., m, n, o, p, R¹, R², R³, R⁴. R⁵, R⁶, R⁷, R⁸, R¹⁰) are as described in the first embodiment, or any aspect thereof.

In a first aspect of the second embodiment, the compound has the following structural formula:

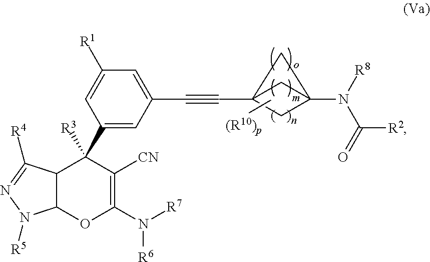

or a pharmaceutically acceptable salt thereof, wherein values for the variables (e.g., m, n, o, p, R¹, R², R³, R⁴. R⁵, R⁶, R⁷, R⁸, R¹⁰) are as described in the first embodiment, or any aspect thereof.

In a second aspect of the second embodiment, the compound has the following structural formula:

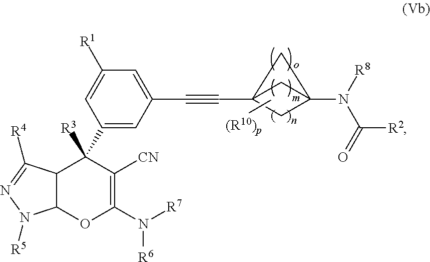

or a pharmaceutically acceptable salt thereof, wherein values for the variables (e.g., m, n, o, p, R¹, R², R³, R⁴. R⁵, R⁶, R⁷, R⁸, R¹⁰) are as described in the first embodiment, or any aspect thereof.

A third embodiment provides a compound of the following structural formula:

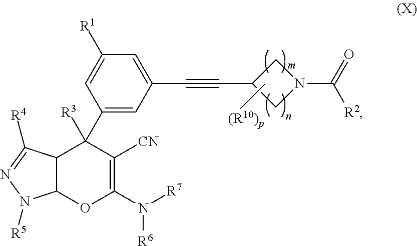

or a pharmaceutically acceptable salt thereof, wherein values for the variables (e.g., m, n, o, p, R¹, R², R³, R⁴. R⁵, R⁶, R⁷, R⁸, R¹⁰) are as described in the first embodiment, or any aspect thereof.

In a first aspect of the third embodiment, the compound has the following structural formula:

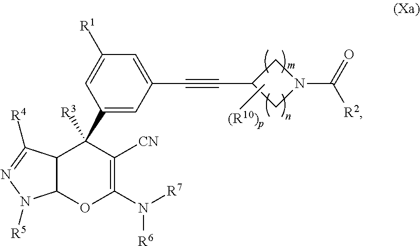

or a pharmaceutically acceptable salt thereof, wherein values for the variables (e.g., m, n, o, p, R¹, R², R³, R⁴. R⁵, R⁶, R⁷, R⁸, R¹⁰) are as described in the first embodiment, or any aspect thereof.

In a second aspect of the third embodiment, the compound has the following structural formula:

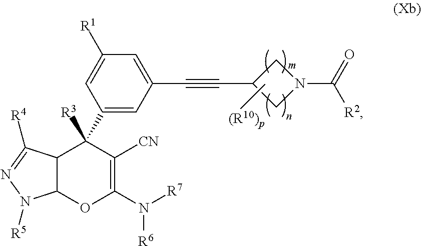

or a pharmaceutically acceptable salt thereof, wherein values for the variables (e.g., m, n, o, p, R¹, R², R³, R⁴. R⁵, R⁶, R⁷, R⁸, R¹⁰) are as described in the first embodiment, or any aspect thereof.

A fourth embodiment provides a compound of the following structural formula:

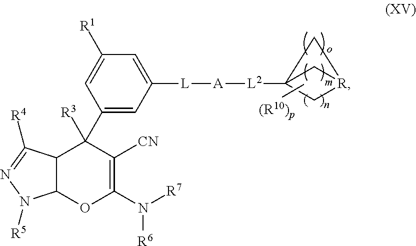

or a pharmaceutically acceptable salt thereof, wherein:
L¹ is an alkylene (e.g., (C₀-C₃)alkylene) or heteroalkylene (e.g., (C₀-C₃)heteroalkylene);
L² is an alkylene (e.g., (C₀-C₃)alkylene) or heteroalkylene (e.g., (C₀-C₃)heteroalkylene);
A is -≡-, cycloalkylene (e.g., (C₃-C₁₂)cycloalkylene), heterocyclylene (e.g., (C₃-C₁₂)heterocyclylene), arylene (e.g., (C₆-C₁₂)arylene) or heteroarylene (e.g., (C₅-C₁₂)heteroarylene); and
values for the remaining variables (e.g., m, n, o, p, R, R¹, R³, R⁴, R⁵, R⁶, R⁷, R¹⁰) are as described in the first or fifth embodiment, or any aspect thereof.

In a first aspect of the fourth embodiment, L¹ is a (C₀-C₁)alkylene (e.g., (C₀)alkylene) or (C₀-C₁)heteroalkylene (e.g., (C₀)heteroalkylene). It will be understood that when L¹ is a (C₀)alkylene or (C₀)heteroalkylene, L¹ is effectively absent, and A is directly linked to the phenyl ring bearing variable R¹ via a covalent bond. Values for the remaining variables are as described in the first or fifth embodiment, or any aspect thereof, or the fourth embodiment.

In a second aspect of the fourth embodiment, L² is a (C₀-C₁)alkylene (e.g., (C₀)alkylene) or (C₀-C₁)heteroalkylene (e.g., (C₀)heteroalkylene). It will be understood that when L² is (C₀)alkylene or (C₀)heteroalkylene, L² is effectively absent, and A is directly linked to the cyclic structure including variable R via a covalent bond. Values for the remaining variables are as described in the first or fifth embodiment, or any aspect thereof, or the fourth embodiment, or first aspect thereof.

In a third aspect of the fourth embodiment, A is -≡-. Values for the remaining variables are as described in the first or fifth embodiment, or any aspect thereof, or the fourth embodiment, or first or second aspect thereof.

In a fourth aspect of the fourth embodiment, A is (C₆-C₁₂)arylene (e.g., phenylene). Values for the remaining variables are as described in the first or fifth embodiment, or any aspect thereof, or the fourth embodiment, or first through third aspects thereof.

In a fifth aspect of the fourth embodiment, A is (C₆-C₁₂)heteroarylene (e.g., pyridinylene, pyrazinylene, pyrimidinylene). Values for the remaining variables are as described in the first or fifth embodiment, or any aspect thereof, or the fourth embodiment, or first through fourth aspects thereof.

In a sixth aspect of the fourth embodiment, A is (C₃-C₁₂)cycloalkylene (e.g., (C₃-C₈)cylcoalkylene). Values for the remaining variables are as described in the first embodiment, or any aspect thereof, or the fourth or fifth embodiment, or first through fifth aspects thereof.

In a seventh aspect of the fourth embodiment, the compound has the following structural formula:

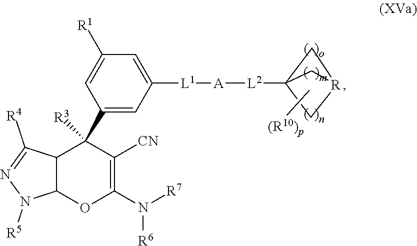

or a pharmaceutically acceptable salt thereof, wherein values for the variables (e.g., m, n, o, p, A, L¹, L², R, R¹, R³, R⁴. R⁵, R⁶, R⁷, R¹⁰) are as described in the first or fifth embodiment, or any aspect thereof, or the fourth embodiment, or first through sixth aspects thereof.

In an eighth aspect of the fourth embodiment, the compound has the following structural formula:

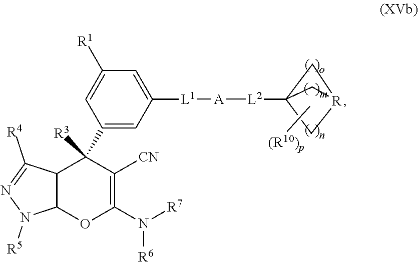

or a pharmaceutically acceptable salt thereof, wherein values for the variables (e.g., m, n, o, p, A, L¹, L², R, R¹, R³, R⁴. R⁵, R⁶, R⁷, R¹⁰) are as described in the first or fifth embodiment, or any aspect thereof, or the fourth embodiment, or first through sixth aspects thereof.

A fifth embodiment provides a compound of structural formula (I), (Ia) or (Ib), or a pharmaceutically acceptable salt thereof, wherein R is —C(H)(N(R⁸)C(O)R²)—, —C(CH₃)(N(R⁸)C(O)R²)—, —C(H)(N(R⁸)CH₂C(O)R²)—, —C(H)(C(O)R²)—, —N(C(O)R²)—, —C(H)(CH₂N(R⁸)C(O)R²)—, —C(H)(CH₂OC(O)R²)—, —N(CH₂C(O)R²)—, —C(H)(hydroxy(C₁-C₁₀)alkyl)-, —C(CH₃)(OH)—, —N(hydroxy(C₁-C₁₀)alkyl)-, —C(CH₃)(OC(O)(C₁-C₁₀)alkyl)-, —C(O)—, —N(H)—, —O—, —C(H)((C₅-C₆)heteroaryl)-, —C(H)(N(R⁸)(C₅-C₆)heteroaryl)-, —C(H)((C₃-C₇)heterocyclyl)- or —N((C₅-C₆)heteroaryl)- when o is 0, and —C(N(R⁸)C(O)R²)—, —CCH₂(N(R⁸)C(O)R²)—, —C(N(R⁸)CH₂C(O)R²)—, —C(C(O)NR^{a}R^{b})—, —C(hydroxy(C₁-C₁₀)alkyl)-, —C((C₅-C₆)heteroaryl)-, —C(N(R⁸)(C₅-C₆)heteroaryl)- or —C((C₃-C₇)heterocyclyl)- when o is other than 0, wherein the heteroaryl can be substituted with one or more R¹¹, and the heterocyclyl can be substituted with one or more substituents selected from oxo or R¹¹. Values for the remaining variables are as described in the first embodiment, or any aspect thereof.

In a first aspect of the fifth embodiment, R is —C(H)(N(R⁸)C(O)R²)—, —C(CH₃)(N(R⁸)C(O)R²)—, —C(H)(C(O)R²)—, —N(C(O)R²)—, —C(H)(CH₂N(R⁸)C(O)R²)—, —C(H)(CH₂OC(O)R²)—, —C(H)(hydroxy(C₁-C₁₀)alkyl)-, —N(hydroxy(C₁-C₁₀)alkyl)-, —C(CH₃)(OC(O)(C₁-C₁₀)alkyl)-, —C(O)— or —N((C₅-C₆)heteroaryl)- when o is 0, and —C(N(R⁸)C(O)R²)— or —C((C₅-C₆)heteroaryl)- when o is other than 0, wherein the heteroaryl can be substituted with one or more R¹¹. Values for the remaining variables are as described in the first embodiment, or any aspect thereof.

In a second aspect of the fifth embodiment, R is —C(H)(N(R⁸)C(O)R²)—, —C(CH₃)(N(R⁸)C(O)R²)—, —C(H)(C(O)R²)— or —N(C(O)R²)— when o is 0, and —C(N(R⁸)C(O)R²)—when o is other than 0. Values for the remaining variables are as described in the first embodiment, or any aspect thereof.

In a third aspect of the fifth embodiment, R is —C(H)((C₅-C₆)heteroaryl)- or —N((C₅-C₆)heteroaryl)- when o is 0, and —C((C₅-C₆)heteroaryl)- when o is other than 0, wherein the heteroaryl can be substituted with one or more R¹¹. Values for the remaining variables are as described in the first embodiment, or any aspect thereof.

In a fourth aspect of the fifth embodiment, R is —C(H)(N(R⁸)C(O)R²)—, —C(CH₃)(N(R′)C(O)R²)—, —C(H)(N(R⁸)CH₂C(O)R²)—, —C(H)(C(O)R²)—, —N(C(O)R²)—, —C(H)(CH₂N(R′)C(O)R²)—, —C(H)(CH₂OC(O)R²)— or —N(CH₂C(O)R²)— when o is 0, and —C(N(R⁸)C(O)R²)—, —CCH₂(N(R⁸)C(O)R²)— or —C(N(R⁸)CH₂C(O)R²)— when o is other than 0. Values for the remaining variables are as described in the first embodiment, or any aspect thereof.

Representative examples of compounds of the structural formulas depicted herein (e.g., structural formulas (I), (Ia) and (Ib)) are provided in Table 1. In Table 1, “D” indicates a t_{1/2} of less than 5 minutes; “C” indicates a t_{1/2} of from 5 minutes to less than 10 minutes; “B” indicates a t_{1/2} of from 10 minutes to less than 20 minutes; and “A” indicates a t_{1/2} of 20 minutes or greater. In Table 1, “D′” indicates a CLᵢₙₜ of greater than 250 μL/min/mg protein; “C′” indicates a CLᵢₙₜ of greater than 100 μL/min/mg protein to 250 μL/min/mg protein; “B′” indicates a relative CLᵢₙₜ of greater than 70 μL/min/mg protein to 100 μL/min/mg protein; and “A′” indicates a relative CLᵢₙₜ of less than or equal to 70 μL/min/mg protein. In Table 1, “D′” indicates an IC₅₀ of greater than 5 μM; “C′” indicates an IC₅₀ of greater than 1 μM to 5 μM; “B′” indicates an IC₅₀ of from 100 nM to 1 μM; “A′” indicates an IC₅₀ of less than 100 nM, In Table 1, “D*” indicates an IC₅₀ of greater than 5 μM; “A*” indicates an IC₅₀ of less than 100 nM.

TABLE 1

| Cmpd | | | Relative | | + |
|---|---|---|---|---|---|
| ID | Compound (Cmpd) Structure | t_{1/2} | Clᵢₙₜ | IC₅₀ | Formate |
| KDG- 0136 | 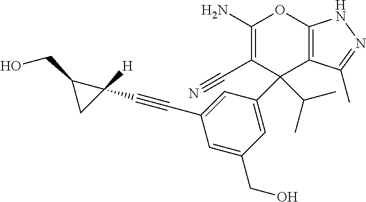 [SMILES: Cc1n[nH]c2c1C(c1cc(C#C[C@@H]3C[C@H]3CO)cc(CO)c1)(C(C)C)C(C#N)=C(N)O2] | B | A′ | C″ | D* |
| KDG- 0320 | 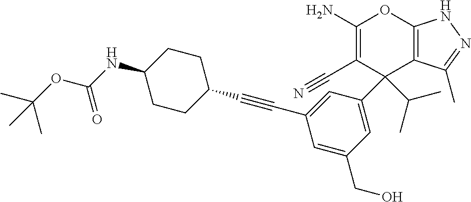 [SMILES: Cc1n[nH]c2c1C(c1cc(C#C[C@H]3CC[C@H](NC(=O)OC(C)(C)C)CC3)cc(CO)c1)(C(C)C)C(C#N)=C(N)O2] | A | A′ | B″ | A* |
| KDG- 0301 | 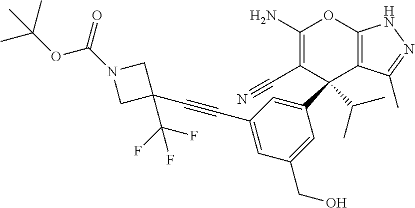 [SMILES: Cc1n[nH]c2c1[C@](c1cc(C#CC3(C(F)(F)F)CN(C(=O)OC(C)(C)C)C3)cc(CO)c1)(C(C)C)C(C#N)=C(N)O2] | A | A′ | A″ | D* |
| KDG- 0385 | 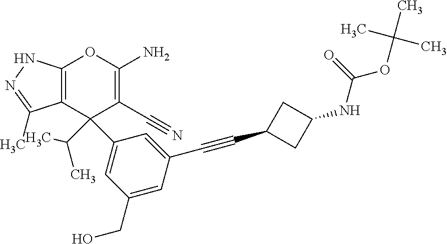 [SMILES: Cc1n[nH]c2c1C(c1cc(C#C[C@H]3C[C@H](NC(=O)OC(C)(C)C)C3)cc(CO)c1)(C(C)C)C(C#N)=C(N)O2] | | | B″ | |
| KDG- 0388 | 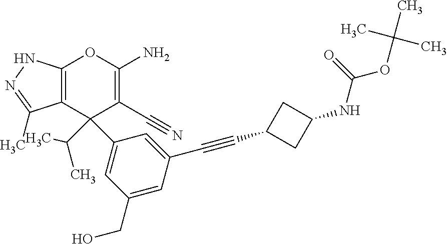 [SMILES: Cc1n[nH]c2c1C(c1cc(C#C[C@H]3C[C@@H](NC(=O)OC(C)(C)C)C3)cc(CO)c1)(C(C)C)C(C#N)=C(N)O2] | A | A′ | B″ | D* |
| KDG- 0388- 0pk2 | 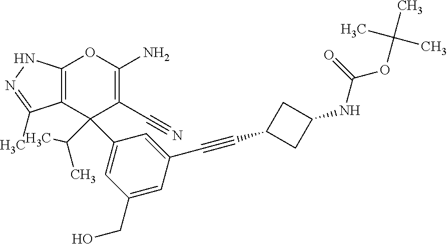 [SMILES: Cc1n[nH]c2c1C(c1cc(C#C[C@H]3C[C@@H](NC(=O)OC(C)(C)C)C3)cc(CO)c1)(C(C)C)C(C#N)=C(N)O2] | | | A″ | D* |
| KDG- 0388- Opk1 | 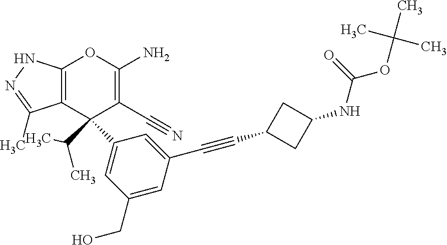 [SMILES: Cc1n[nH]c2c1[C@](c1cc(C#C[C@H]3C[C@@H](NC(=O)OC(C)(C)C)C3)cc(CO)c1)(C(C)C)C(C#N)=C(N)O2] | | | D″ | A* |
| KDG- 0271 | 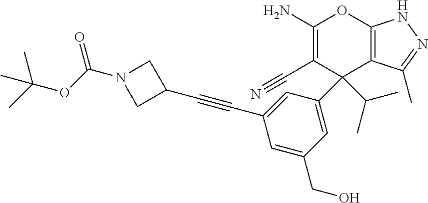 [SMILES: Cc1n[nH]c2c1C(c1cc(C#CC3CN(C(=O)OC(C)(C)C)C3)cc(CO)c1)(C(C)C)C(C#N)=C(N)O2] | B | B′ | A″ | D* |
| KDG- 0271- 0-pk2 | 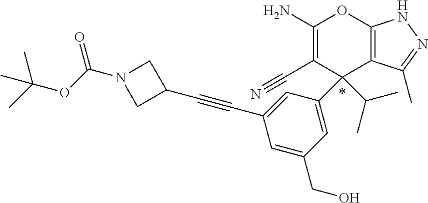 [SMILES: Cc1n[nH]c2c1C(c1cc(C#CC3CN(C(=O)OC(C)(C)C)C3)cc(CO)c1)(C(C)C)C(C#N)=C(N)O2] | | | D″ | |
| KDG- 0271- 0-pk1 | 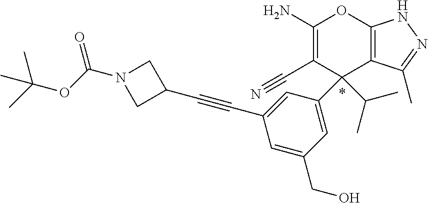 [SMILES: Cc1n[nH]c2c1C(c1cc(C#CC3CN(C(=O)OC(C)(C)C)C3)cc(CO)c1)(C(C)C)C(C#N)=C(N)O2] | | | A″ | |
| KDG- 0271- 200 | 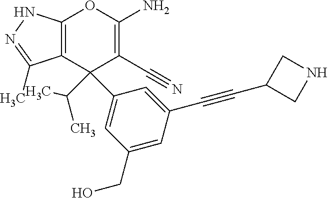 [SMILES: Cc1n[nH]c2c1C(c1cc(C#CC3CNC3)cc(CO)c1)(C(C)C)C(C#N)=C(N)O2] | | | D″ | |
| KDG- 0412 | 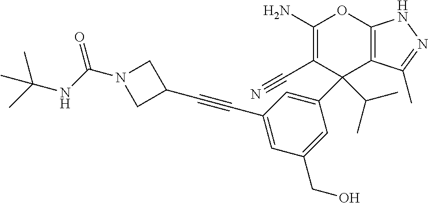 [SMILES: Cc1n[nH]c2c1C(c1cc(C#CC3CN(C(=O)NC(C)(C)C)C3)cc(CO)c1)(C(C)C)C(C#N)=C(N)O2] | B | B′ | D″ | D* |
| KDG- 0413 | 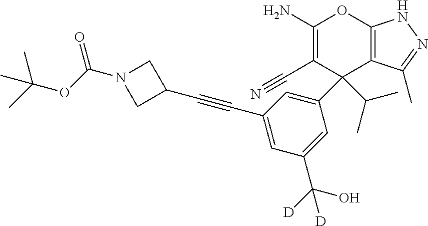 [SMILES: [2H]C([2H])(O)c1cc(C#CC2CN(C(=O)OC(C)(C)C)C2)cc(C2(C(C)C)C(C#N)=C(N)Oc3[nH]nc(C)c32)c1] | B | B′ | B″ | A* |
| KDG- 0297 | 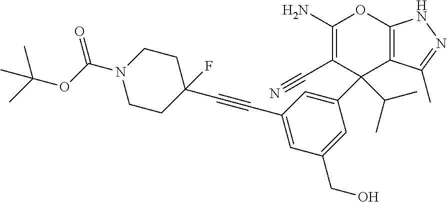 [SMILES: Cc1n[nH]c2c1C(c1cc(C#CC3(F)CCN(C(=O)OC(C)(C)C)CC3)cc(CO)c1)(C(C)C)C(C#N)=C(N)O2] | C | C′ | B″ | D* |
| KDG- 0345 | 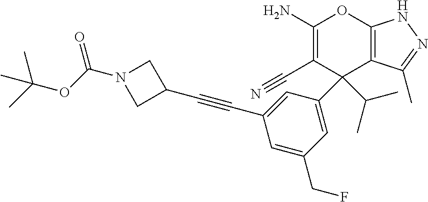 [SMILES: Cc1n[nH]c2c1C(c1cc(C#CC3CN(C(=O)OC(C)(C)C)C3)cc(CF)c1)(C(C)C)C(C#N)=C(N)O2] | C | C′ | A″ | |
| KDG- 0296 | 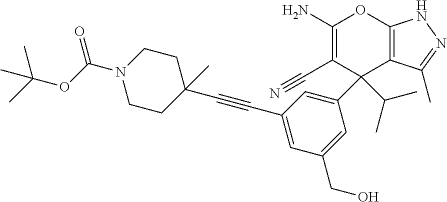 [SMILES: Cc1n[nH]c2c1C(c1cc(C#CC3(C)CCN(C(=O)OC(C)(C)C)CC3)cc(CO)c1)(C(C)C)C(C#N)=C(N)O2] | C | C′ | C″ | A* |
| KDG- 0289 | 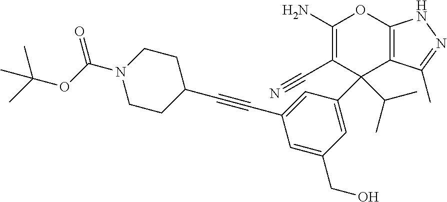 [SMILES: Cc1n[nH]c2c1C(c1cc(C#CC3CCN(C(=O)OC(C)(C)C)CC3)cc(CO)c1)(C(C)C)C(C#N)=C(N)O2] | C | C′ | A″ | A* |
| KDG- 0300 | 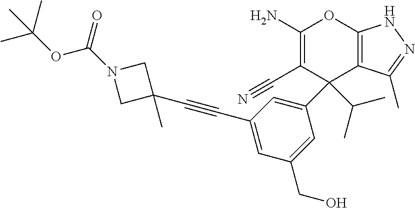 [SMILES: Cc1n[nH]c2c1C(c1cc(C#CC3(C)CN(C(=O)OC(C)(C)C)C3)cc(CO)c1)(C(C)C)C(C#N)=C(N)O2] | C | D′ | | |
| KDG- 0261- 100 | 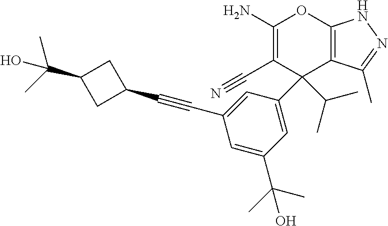 [SMILES: Cc1n[nH]c2c1C(c1cc(C#C[C@H]3C[C@@H](C(C)(C)O)C3)cc(C(C)(C)O)c1)(C(C)C)C(C#N)=C(N)O2] | D | D′ | | D* |
| KDG- 0258 | 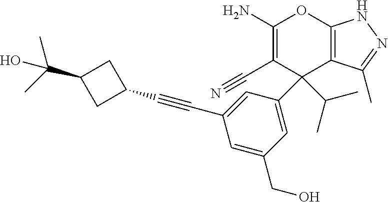 [SMILES: Cc1n[nH]c2c1C(c1cc(C#C[C@H]3C[C@H](C(C)(C)O)C3)cc(CO)c1)(C(C)C)C(C#N)=C(N)O2] | D | D′ | A″ | |
| KDG- 0258- 100 | 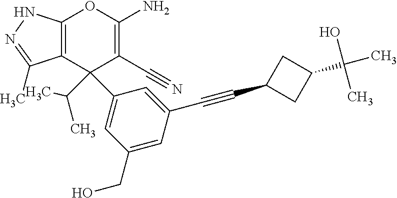 [SMILES: Cc1n[nH]c2c1C(c1cc(C#C[C@H]3C[C@H](C(C)(C)O)C3)cc(CO)c1)(C(C)C)C(C#N)=C(N)O2] | | | B″ | |
| KDG- 0260- 100 | 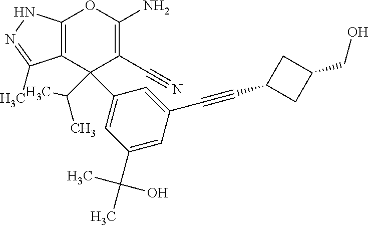 [SMILES: Cc1n[nH]c2c1C(c1cc(C#C[C@H]3C[C@@H](CO)C3)cc(C(C)(C)O)c1)(C(C)C)C(C#N)=C(N)O2] | | | D″ | |
| KDG- 0261 | 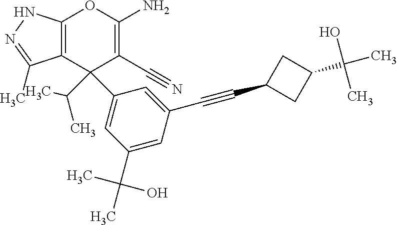 [SMILES: Cc1n[nH]c2c1C(c1cc(C#C[C@H]3C[C@H](C(C)(C)O)C3)cc(C(C)(C)O)c1)(C(C)C)C(C#N)=C(N)O2] | | | C″ | |
| KDG- 0261- 100 | 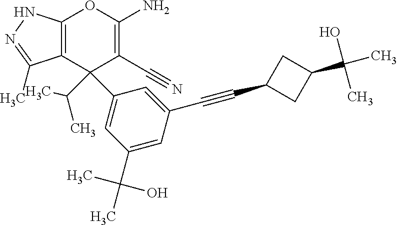 [SMILES: Cc1n[nH]c2c1C(c1cc(C#C[C@H]3C[C@@H](C(C)(C)O)C3)cc(C(C)(C)O)c1)(C(C)C)C(C#N)=C(N)O2] | D | | C″ | |
| KDG- 0262 | 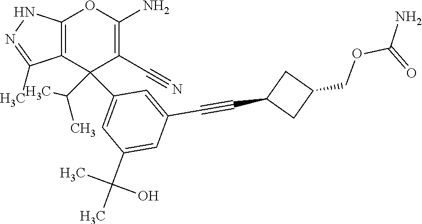 [SMILES: Cc1n[nH]c2c1C(c1cc(C#C[C@H]3C[C@H](COC(N)=O)C3)cc(C(C)(C)O)c1)(C(C)C)C(C#N)=C(N)O2] | D | | C″ | |
| KDG- 0262- 100 | 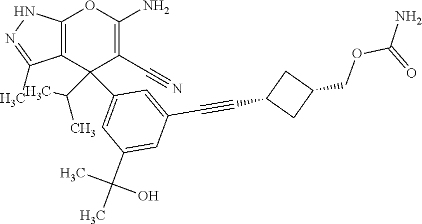 [SMILES: Cc1n[nH]c2c1C(c1cc(C#C[C@H]3C[C@@H](COC(N)=O)C3)cc(C(C)(C)O)c1)(C(C)C)C(C#N)=C(N)O2] | | | C″ | |
| KDG- 0265 | 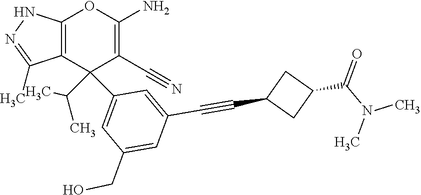 [SMILES: Cc1n[nH]c2c1C(c1cc(C#C[C@H]3C[C@H](C(=O)N(C)C)C3)cc(CO)c1)(C(C)C)C(C#N)=C(N)O2] | C | | C″ | |
| KDG- 0265- 100 | 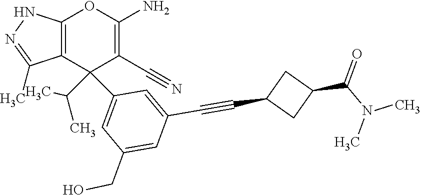 [SMILES: Cc1n[nH]c2c1C(c1cc(C#C[C@H]3C[C@@H](C(=O)N(C)C)C3)cc(CO)c1)(C(C)C)C(C#N)=C(N)O2] | | | B″ | |
| KDG- 0266 | 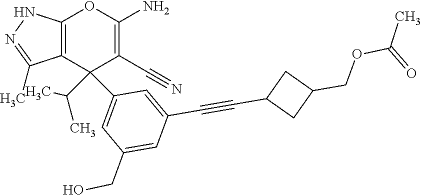 [SMILES: CC(=O)OCC1CC(C#Cc2cc(CO)cc(C3(C(C)C)C(C#N)=C(N)Oc4[nH]nc(C)c43)c2)C1] | | | B″ | |
| KDG- 0268 | 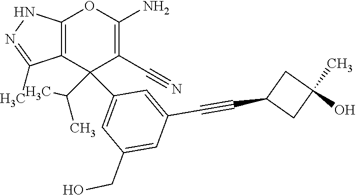 [SMILES: Cc1n[nH]c2c1C(c1cc(C#C[C@H]3C[C@@](C)(O)C3)cc(CO)c1)(C(C)C)C(C#N)=C(N)O2] | | | C″ | |
| KDG- 0269 | 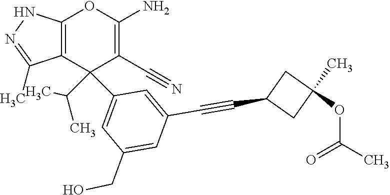 [SMILES: CC(=O)O[C@]1(C)C[C@H](C#Cc2cc(CO)cc(C3(C(C)C)C(C#N)=C(N)Oc4[nH]nc(C)c43)c2)C1] | | | B″ | |
| KDG- 0270 | 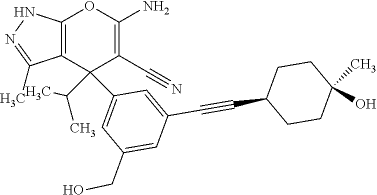 [SMILES: Cc1n[nH]c2c1C(c1cc(C#C[C@H]3CC[C@@](C)(O)CC3)cc(CO)c1)(C(C)C)C(C#N)=C(N)O2] | D | | B″ | |
| KDG- 0270- 100 | 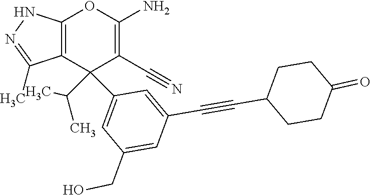 [SMILES: Cc1n[nH]c2c1C(c1cc(C#CC3CCC(=O)CC3)cc(CO)c1)(C(C)C)C(C#N)=C(N)O2] | | | B″ | |
| KDG- 0272 | 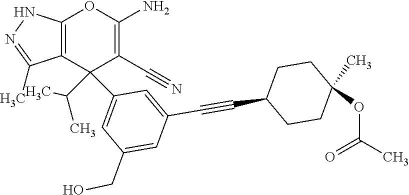 [SMILES: CC(=O)O[C@]1(C)CC[C@H](C#Cc2cc(CO)cc(C3(C(C)C)C(C#N)=C(N)Oc4[nH]nc(C)c43)c2)CC1] | | | A″ | |
| KDG- 0273 | 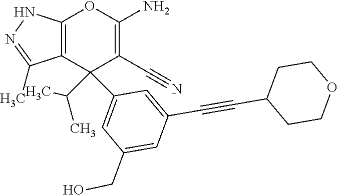 [SMILES: Cc1n[nH]c2c1C(c1cc(C#CC3CCOCC3)cc(CO)c1)(C(C)C)C(C#N)=C(N)O2] | D | | B″ | |
| KDG- 0280 | 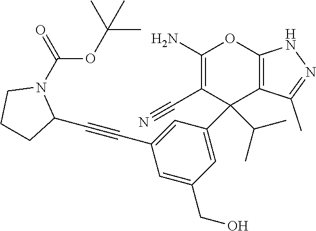 [SMILES: Cc1n[nH]c2c1C(c1cc(C#CC3CCCN3C(=O)OC(C)(C)C)cc(CO)c1)(C(C)C)C(C#N)=C(N)O2] | D | D′ | B″ | D* |
| KDG- 0281 | 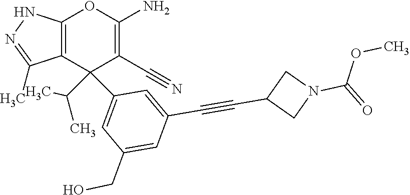 [SMILES: COC(=O)N1CC(C#Cc2cc(CO)cc(C3(C(C)C)C(C#N)=C(N)Oc4[nH]nc(C)c43)c2)C1] | | | B″ | |
| KDG- 0284 | 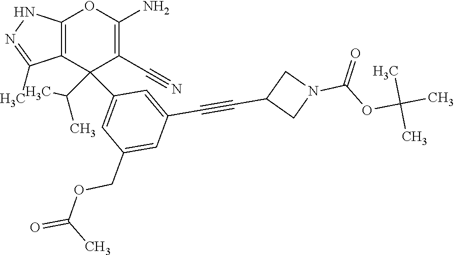 [SMILES: CC(=O)OCc1cc(C#CC2CN(C(=O)OC(C)(C)C)C2)cc(C2(C(C)C)C(C#N)=C(N)Oc3[nH]nc(C)c32)c1] | | | B″ | |
| KDG- 0287 | 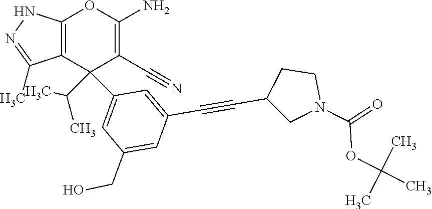 [SMILES: Cc1n[nH]c2c1C(c1cc(C#CC3CCN(C(=O)OC(C)(C)C)C3)cc(CO)c1)(C(C)C)C(C#N)=C(N)O2] | | | B″ | |
| KDG- 0288 | 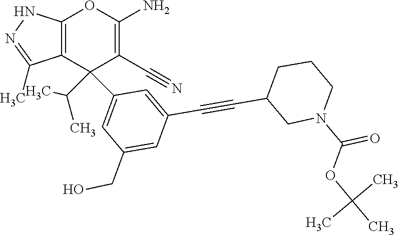 [SMILES: Cc1n[nH]c2c1C(c1cc(C#CC3CCCN(C(=O)OC(C)(C)C)C3)cc(CO)c1)(C(C)C)C(C#N)=C(N)O2] | | | C″ | |
| KDG- 0293 | 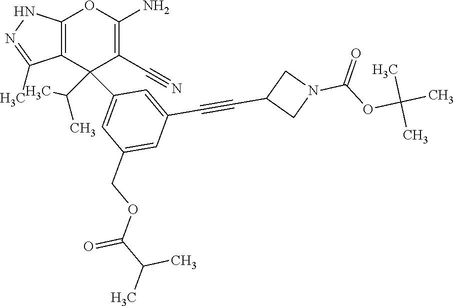 [SMILES: Cc1n[nH]c2c1C(c1cc(C#CC3CN(C(=O)OC(C)(C)C)C3)cc(COC(=O)C(C)C)c1)(C(C)C)C(C#N)=C(N)O2] | | | A″ | |
| KDG- 0294 | 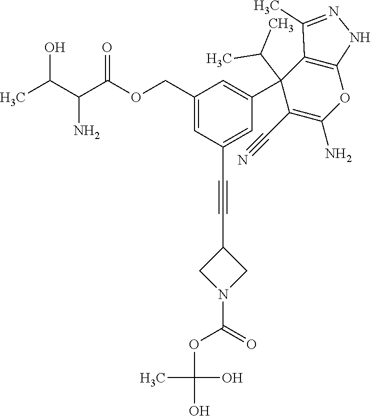 [SMILES: Cc1n[nH]c2c1C(c1cc(C#CC3CN(C(=O)OC(C)(O)O)C3)cc(COC(=O)C(N)C(C)O)c1)(C(C)C)C(C#N)=C(N)O2] | | | B″ | |
| KDG- 0304 | 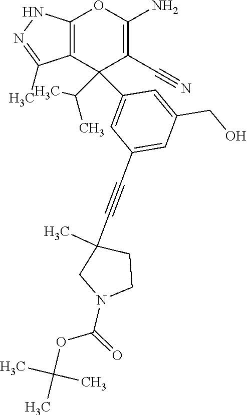 [SMILES: Cc1n[nH]c2c1C(c1cc(C#CC3(C)CCN(C(=O)OC(C)(C)C)C3)cc(CO)c1)(C(C)C)C(C#N)=C(N)O2] | | | B″ | |
| KDG- 0308 | 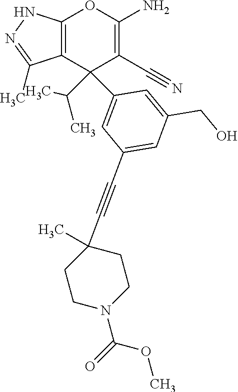 [SMILES: COC(=O)N1CCC(C)(C#Cc2cc(CO)cc(C3(C(C)C)C(C#N)=C(N)Oc4[nH]nc(C)c43)c2)CC1] | | | B″ | |
| KDG- 0313 | 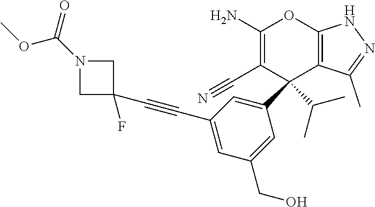 [SMILES: COC(=O)N1CC(F)(C#Cc2cc(CO)cc([C@@]3(C(C)C)C(C#N)=C(N)Oc4[nH]nc(C)c43)c2)C1] | A | A′ | B″ | D* |
| KDG- 0390 | 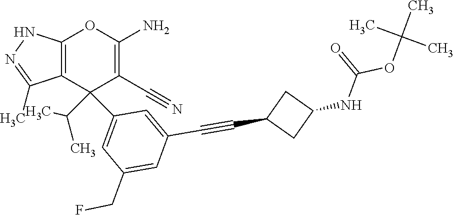 [SMILES: Cc1n[nH]c2c1C(c1cc(C#C[C@H]3C[C@H](NC(=O)OC(C)(C)C)C3)cc(CF)c1)(C(C)C)C(C#N)=C(N)O2] | | | B″ | |
| KDG- 0393 | 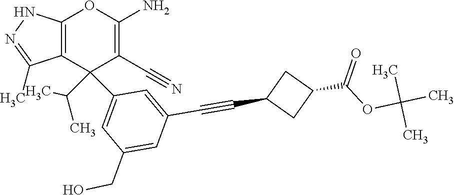 [SMILES: Cc1n[nH]c2c1C(c1cc(C#C[C@H]3C[C@H](C(=O)OC(C)(C)C)C3)cc(CO)c1)(C(C)C)C(C#N)=C(N)O2] | D | | | |
| KDG- 0395 | 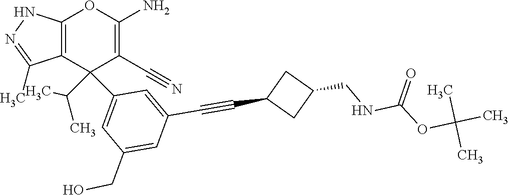 [SMILES: Cc1n[nH]c2c1C(c1cc(C#C[C@H]3C[C@H](CNC(=O)OC(C)(C)C)C3)cc(CO)c1)(C(C)C)C(C#N)=C(N)O2] | | | B″ | |
| KDG- 0395- 100 | 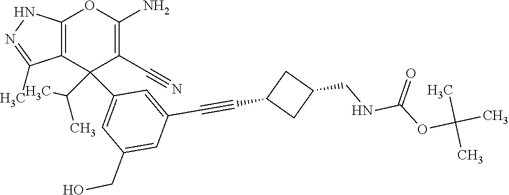 [SMILES: Cc1n[nH]c2c1C(c1cc(C#C[C@H]3C[C@@H](CNC(=O)OC(C)(C)C)C3)cc(CO)c1)(C(C)C)C(C#N)=C(N)O2] | | | B″ | |
| KDG- 0411 | 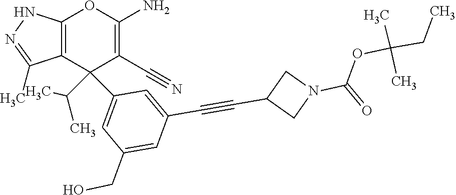 [SMILES: CCC(C)(C)OC(=O)N1CC(C#Cc2cc(CO)cc(C3(C(C)C)C(C#N)=C(N)Oc4[nH]nc(C)c43)c2)C1] | | | B″ | |
| KDG- 0416 | 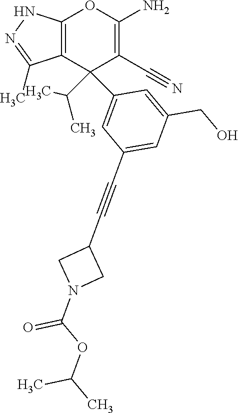 [SMILES: Cc1n[nH]c2c1C(c1cc(C#CC3CN(C(=O)OC(C)C)C3)cc(CO)c1)(C(C)C)C(C#N)=C(N)O2] | | | A″ | |
| KDG- 0418 | 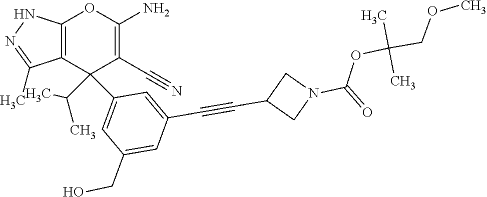 [SMILES: COCC(C)(C)OC(=O)N1CC(C#Cc2cc(CO)cc(C3(C(C)C)C(C#N)=C(N)Oc4[nH]nc(C)c43)c2)C1] | | | B″ | |
| KDG- 0472 | 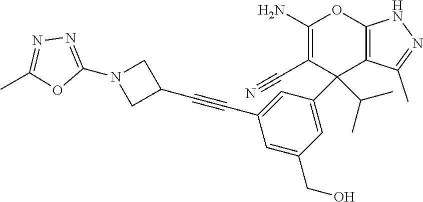 [SMILES: Cc1nnc(N2CC(C#Cc3cc(CO)cc(C4(C(C)C)C(C#N)=C(N)Oc5[nH]nc(C)c54)c3)C2)o1] | A | A′ | C″ | A* |
| KDG- 0478 | 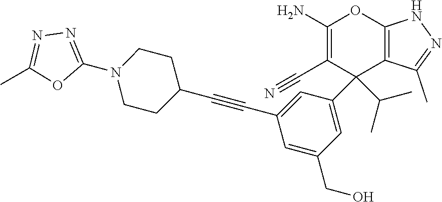 [SMILES: Cc1nnc(N2CCC(C#Cc3cc(CO)cc(C4(C(C)C)C(C#N)=C(N)Oc5[nH]nc(C)c54)c3)CC2)o1] | C | B′ | C″ | D* |
| KDG- 0321 | 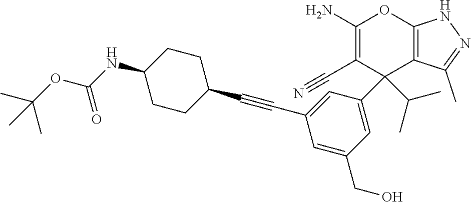 [SMILES: Cc1n[nH]c2c1C(c1cc(C#C[C@H]3CC[C@@H](NC(=O)OC(C)(C)C)CC3)cc(CO)c1)(C(C)C)C(C#N)=C(N)O2] | D | D′ | A″ | A* |
| KDG- 0502 | 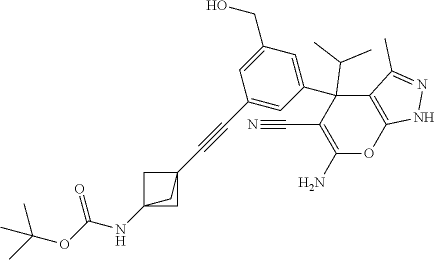 [SMILES: Cc1n[nH]c2c1C(c1cc(C#CC34CC(NC(=O)OC(C)(C)C)(C3)C4)cc(CO)c1)(C(C)C)C(C#N)=C(N)O2] | A | A′ | B″ | A* |
| KDG- 0502- P1 | 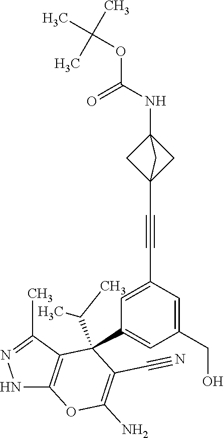 [SMILES: Cc1n[nH]c2c1[C@](c1cc(C#CC34CC(NC(=O)OC(C)(C)C)(C3)C4)cc(CO)c1)(C(C)C)C(C#N)=C(N)O2] | | | | |
| KDG- 0502- P2 | 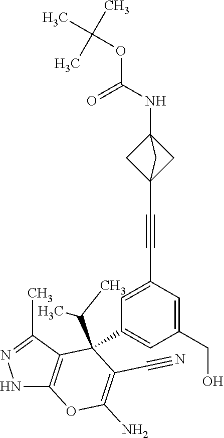 [SMILES: Cc1n[nH]c2c1[C@@](c1cc(C#CC34CC(NC(=O)OC(C)(C)C)(C3)C4)cc(CO)c1)(C(C)C)C(C#N)=C(N)O2] | | | | |
| KDG- 0453 | 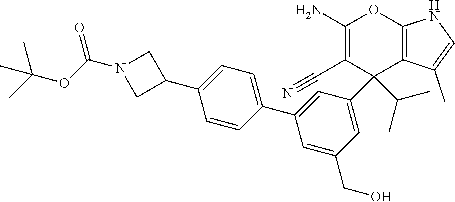 [SMILES: Cc1c[nH]c2c1C(c1cc(CO)cc(-c3ccc(C4CN(C(=O)OC(C)(C)C)C4)cc3)c1)(C(C)C)C(C#N)=C(N)O2] | B | B′ | A″ | A* |
| KDG- 0347 | 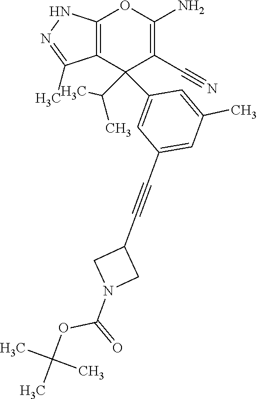 [SMILES: Cc1cc(C#CC2CN(C(=O)OC(C)(C)C)C2)cc(C2(C(C)C)C(C#N)=C(N)Oc3[nH]nc(C)c32)c1] | | | | |
| KDG- 0383 | 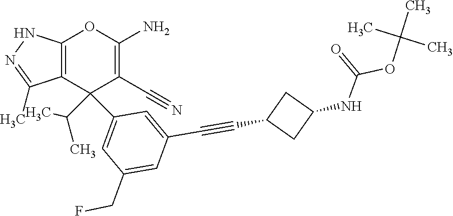 [SMILES: Cc1n[nH]c2c1C(c1cc(C#C[C@H]3C[C@@H](NC(=O)OC(C)(C)C)C3)cc(CF)c1)(C(C)C)C(C#N)=C(N)O2] | | | B″ | |
| KDG- 0386 | 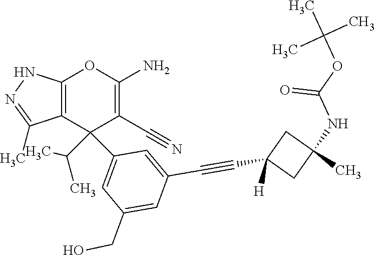 [SMILES: Cc1n[nH]c2c1C(c1cc(C#C[C@H]3C[C@@](C)(NC(=O)OC(C)(C)C)C3)cc(CO)c1)(C(C)C)C(C#N)=C(N)O2] | | | | |
| KDG- 0387 | 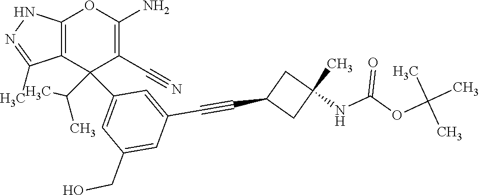 [SMILES: Cc1n[nH]c2c1C(c1cc(C#C[C@H]3C[C@](C)(NC(=O)OC(C)(C)C)C3)cc(CO)c1)(C(C)C)C(C#N)=C(N)O2] | | | B″ | |
| KDG- 0474 | 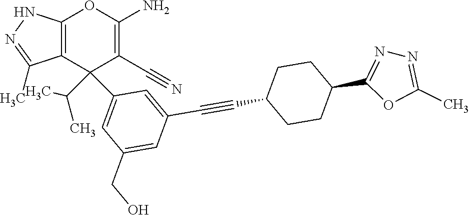 [SMILES: Cc1nnc([C@H]2CC[C@H](C#Cc3cc(CO)cc(C4(C(C)C)C(C#N)=C(N)Oc5[nH]nc(C)c54)c3)CC2)o1] | | | | |
| KDG- 0481- 0 | 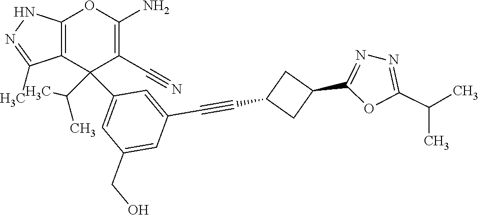 [SMILES: Cc1n[nH]c2c1C(c1cc(C#C[C@H]3C[C@H](c4nnc(C(C)C)o4)C3)cc(CO)c1)(C(C)C)C(C#N)=C(N)O2] | | | | |
| KDG- 0481- 100 | 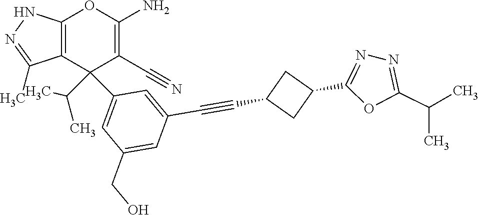 [SMILES: Cc1n[nH]c2c1C(c1cc(C#C[C@H]3C[C@@H](c4nnc(C(C)C)o4)C3)cc(CO)c1)(C(C)C)C(C#N)=C(N)O2] | | | | |
| KDG- 0483- 0 | 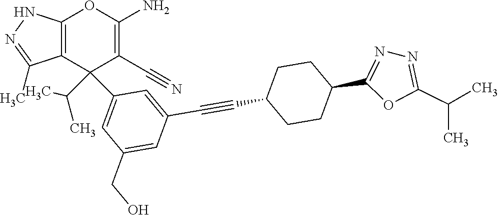 [SMILES: Cc1n[nH]c2c1C(c1cc(C#C[C@H]3CC[C@H](c4nnc(C(C)C)o4)CC3)cc(CO)c1)(C(C)C)C(C#N)=C(N)O2] | | | | |
| KDG- 0483- 200 | 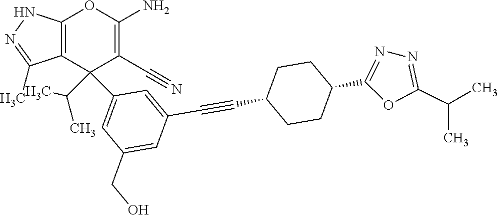 [SMILES: Cc1n[nH]c2c1C(c1cc(C#C[C@H]3CC[C@@H](c4nnc(C(C)C)o4)CC3)cc(CO)c1)(C(C)C)C(C#N)=C(N)O2] | | | | |
| KDG- 0490 | 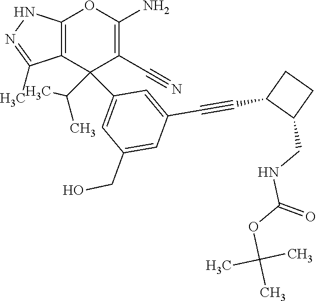 [SMILES: Cc1n[nH]c2c1C(c1cc(C#C[C@@H]3CC[C@@H]3CNC(=O)OC(C)(C)C)cc(CO)c1)(C(C)C)C(C#N)=C(N)O2] | D | | B″ | |
| KDG- 0505 | 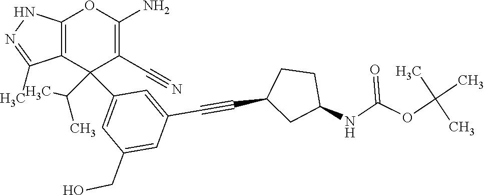 [SMILES: Cc1n[nH]c2c1C(c1cc(C#C[C@H]3CC[C@@H](NC(=O)OC(C)(C)C)C3)cc(CO)c1)(C(C)C)C(C#N)=C(N)O2] | D | | A″ | |
| KDG- 0506 | 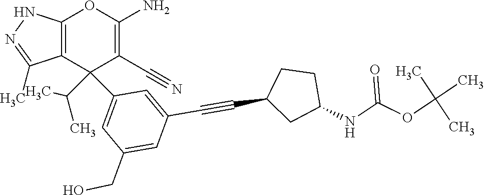 [SMILES: Cc1n[nH]c2c1C(c1cc(C#C[C@H]3CC[C@H](NC(=O)OC(C)(C)C)C3)cc(CO)c1)(C(C)C)C(C#N)=C(N)O2] | C | | B″ | |
| KDG- 0515 | 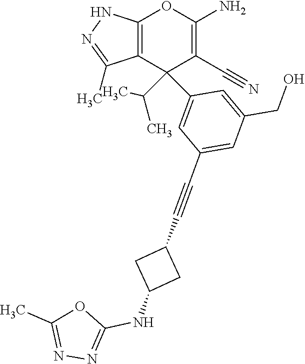 [SMILES: Cc1nnc(N[C@H]2C[C@@H](C#Cc3cc(CO)cc(C4(C(C)C)C(C#N)=C(N)Oc5[nH]nc(C)c54)c3)C2)o1] | | | | |
| KDG- 0516 | 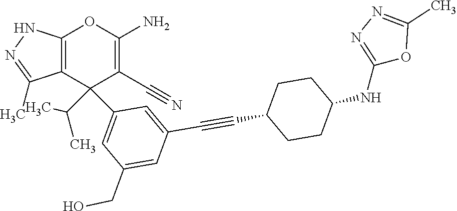 [SMILES: Cc1nnc(N[C@H]2CC[C@@H](C#Cc3cc(CO)cc(C4(C(C)C)C(C#N)=C(N)Oc5[nH]nc(C)c54)c3)CC2)o1] | | | C″ | |
| KDG- 0518 | 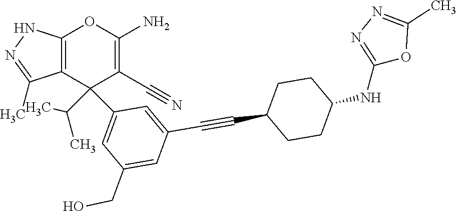 [SMILES: Cc1nnc(N[C@H]2CC[C@H](C#Cc3cc(CO)cc(C4(C(C)C)C(C#N)=C(N)Oc5[nH]nc(C)c54)c3)CC2)o1] | | | C″ | |
| KDG- 0520 | 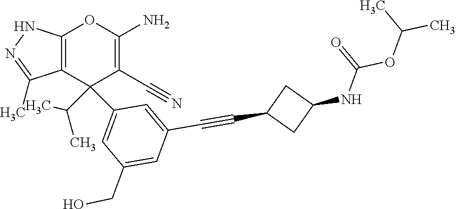 [SMILES: Cc1n[nH]c2c1C(c1cc(C#C[C@H]3C[C@@H](NC(=O)OC(C)C)C3)cc(CO)c1)(C(C)C)C(C#N)=C(N)O2] | | | A″ | |
| KDG- 0521 | 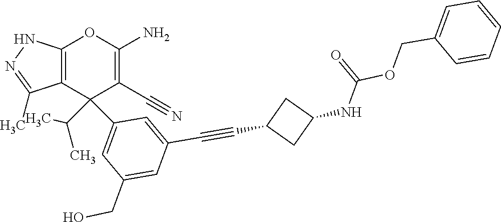 [SMILES: Cc1n[nH]c2c1C(c1cc(C#C[C@H]3C[C@@H](NC(=O)OCc4ccccc4)C3)cc(CO)c1)(C(C)C)C(C#N)=C(N)O2] | | | A″ | |
| KDG- 0522 | 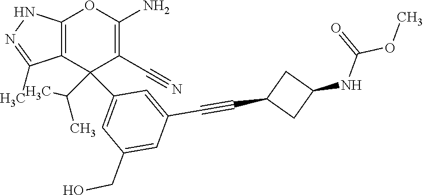 [SMILES: COC(=O)N[C@H]1C[C@@H](C#Cc2cc(CO)cc(C3(C(C)C)C(C#N)=C(N)Oc4[nH]nc(C)c43)c2)C1] | | | B″ | |
| KDG- 0522- 100 | 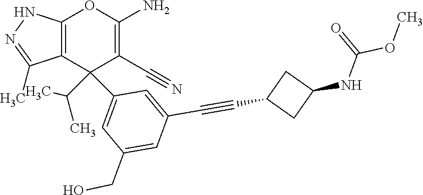 [SMILES: COC(=O)N[C@H]1C[C@H](C#Cc2cc(CO)cc(C3(C(C)C)C(C#N)=C(N)Oc4[nH]nc(C)c43)c2)C1] | | | B″ | |
| KDG- 0523 | 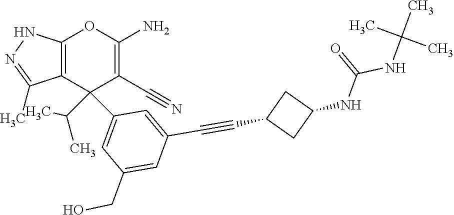 [SMILES: Cc1n[nH]c2c1C(c1cc(C#C[C@H]3C[C@@H](NC(=O)NC(C)(C)C)C3)cc(CO)c1)(C(C)C)C(C#N)=C(N)O2] | | | B″ | |
| KDG- 0524 | 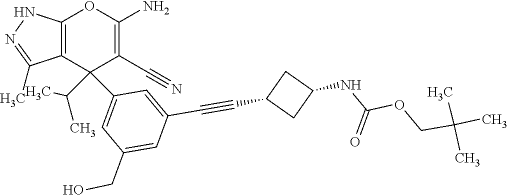 [SMILES: Cc1n[nH]c2c1C(c1cc(C#C[C@H]3C[C@@H](NC(=O)OCC(C)(C)C)C3)cc(CO)c1)(C(C)C)C(C#N)=C(N)O2] | | | B″ | |
| KDG- 0526 | 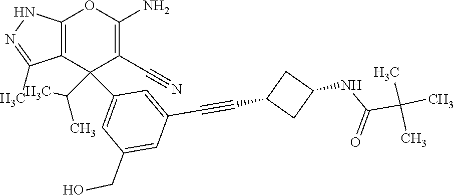 [SMILES: Cc1n[nH]c2c1C(c1cc(C#C[C@H]3C[C@@H](NC(=O)C(C)(C)C)C3)cc(CO)c1)(C(C)C)C(C#N)=C(N)O2] | | | B″ | |
| KDG- 0527 | 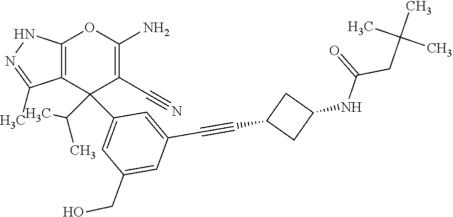 [SMILES: Cc1n[nH]c2c1C(c1cc(C#C[C@H]3C[C@@H](NC(=O)CC(C)(C)C)C3)cc(CO)c1)(C(C)C)C(C#N)=C(N)O2] | | | B″ | |
| KDG- 0530 | 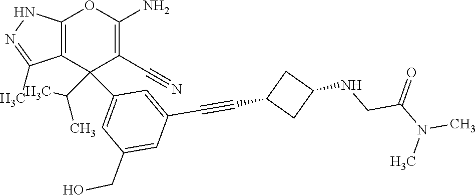 [SMILES: Cc1n[nH]c2c1C(c1cc(C#C[C@H]3C[C@@H](NCC(=O)N(C)C)C3)cc(CO)c1)(C(C)C)C(C#N)=C(N)O2] | | | D″ | |
| KDG- 0543 | 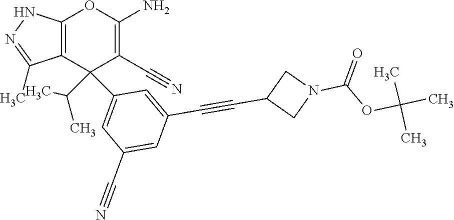 [SMILES: Cc1n[nH]c2c1C(c1cc(C#N)cc(C#CC3CN(C(=O)OC(C)(C)C)C3)c1)(C(C)C)C(C#N)=C(N)O2] | B | | B″ | |
| KDG- 0547 | 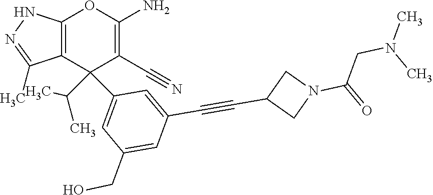 [SMILES: Cc1n[nH]c2c1C(c1cc(C#CC3CN(C(=O)CN(C)C)C3)cc(CO)c1)(C(C)C)C(C#N)=C(N)O2] | | | D″ | |
| KDG- 0554 | 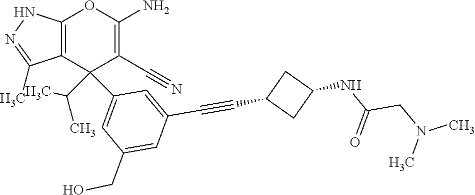 [SMILES: Cc1n[nH]c2c1C(c1cc(C#C[C@H]3C[C@@H](NC(=O)CN(C)C)C3)cc(CO)c1)(C(C)C)C(C#N)=C(N)O2] | | | C″ | |
| KDG- 0555 | 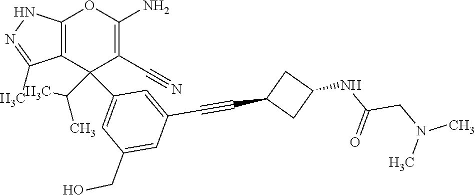 [SMILES: Cc1n[nH]c2c1C(c1cc(C#C[C@H]3C[C@H](NC(=O)CN(C)C)C3)cc(CO)c1)(C(C)C)C(C#N)=C(N)O2] | | | C″ | |
| KDG- 0556 | 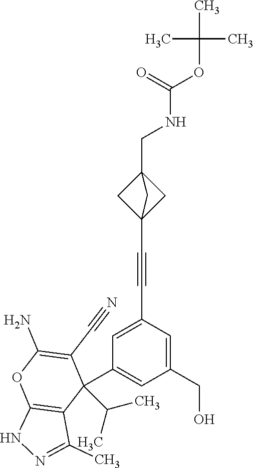 [SMILES: Cc1n[nH]c2c1C(c1cc(C#CC34CC(CNC(=O)OC(C)(C)C)(C3)C4)cc(CO)c1)(C(C)C)C(C#N)=C(N)O2] | | | | |
| KDG- 0558 | 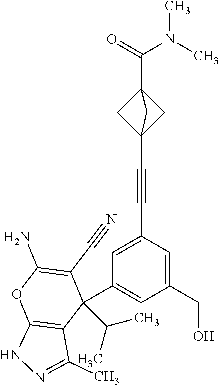 [SMILES: Cc1n[nH]c2c1C(c1cc(C#CC34CC(C(=O)N(C)C)(C3)C4)cc(CO)c1)(C(C)C)C(C#N)=C(N)O2] | | | C″ | |
| KDG- 0571 | 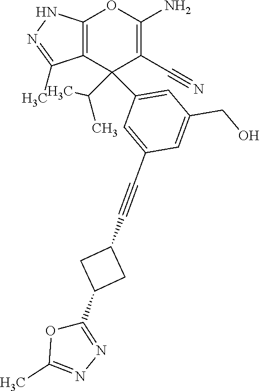 [SMILES: Cc1nnc([C@H]2C[C@@H](C#Cc3cc(CO)cc(C4(C(C)C)C(C#N)=C(N)Oc5[nH]nc(C)c54)c3)C2)o1] | | | | |
| KDG- 0572 | 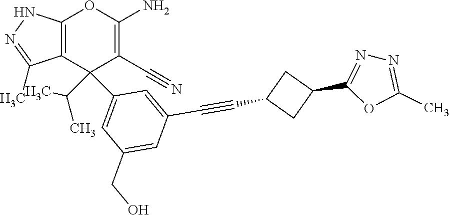 [SMILES: Cc1nnc([C@H]2C[C@H](C#Cc3cc(CO)cc(C4(C(C)C)C(C#N)=C(N)Oc5[nH]nc(C)c54)c3)C2)o1] | | | | |
| KDG- 0573 | 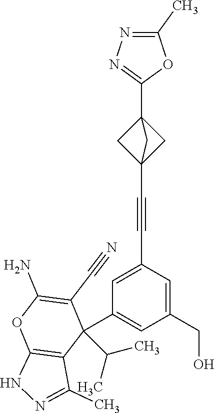 [SMILES: Cc1nnc(C23CC(C#Cc4cc(CO)cc(C5(C(C)C)C(C#N)=C(N)Oc6[nH]nc(C)c65)c4)(C2)C3)o1] | | | B″ | |
| KDG- 0574 | 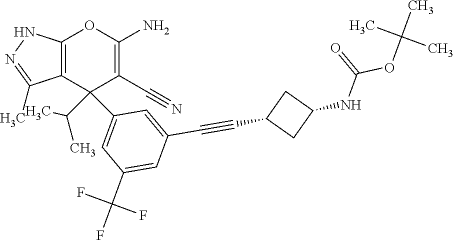 [SMILES: Cc1n[nH]c2c1C(c1cc(C#C[C@H]3C[C@@H](NC(=O)OC(C)(C)C)C3)cc(C(F)(F)F)c1)(C(C)C)C(C#N)=C(N)O2] | | | B″ | |
| KDG- 0575 | 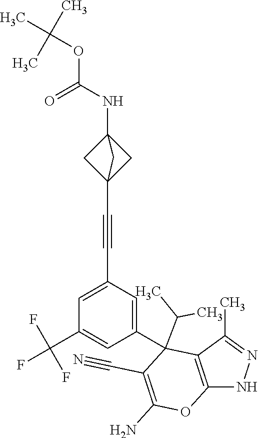 [SMILES: Cc1n[nH]c2c1C(c1cc(C#CC34CC(NC(=O)OC(C)(C)C)(C3)C4)cc(C(F)(F)F)c1)(C(C)C)C(C#N)=C(N)O2] | | | | |
| KDG- 0576 | 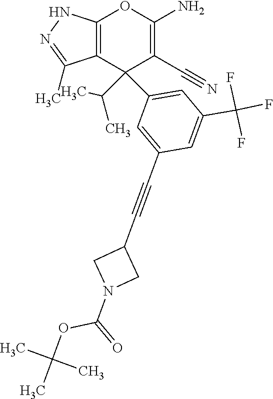 [SMILES: Cc1n[nH]c2c1C(c1cc(C#CC3CN(C(=O)OC(C)(C)C)C3)cc(C(F)(F)F)c1)(C(C)C)C(C#N)=C(N)O2] | | | | |
| KDG- 0627- 0 | 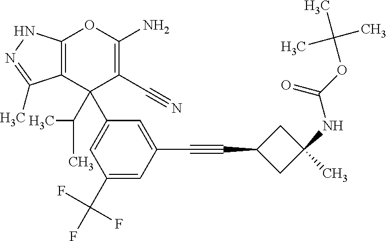 [SMILES: Cc1n[nH]c2c1C(c1cc(C#C[C@H]3C[C@@](C)(NC(=O)OC(C)(C)C)C3)cc(C(F)(F)F)c1)(C(C)C)C(C#N)=C(N)O2] | | | | |
| KDG- 0627- 100 | 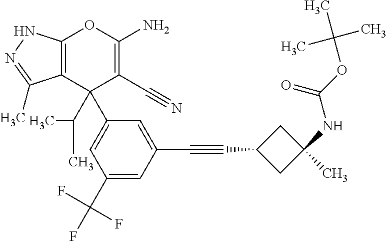 [SMILES: Cc1n[nH]c2c1C(c1cc(C#C[C@H]3C[C@](C)(NC(=O)OC(C)(C)C)C3)cc(C(F)(F)F)c1)(C(C)C)C(C#N)=C(N)O2] | | | | |
| KDG- 0628 | 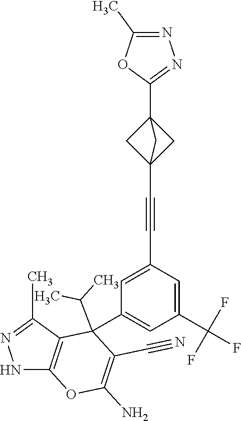 [SMILES: Cc1nnc(C23CC(C#Cc4cc(C(F)(F)F)cc(C5(C(C)C)C(C#N)=C(N)Oc6[nH]nc(C)c65)c4)(C2)C3)o1] | | | | |
| KDG- 0631- 0 | 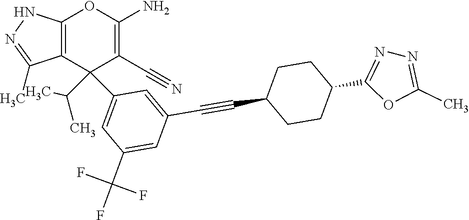 [SMILES: Cc1nnc([C@H]2CC[C@H](C#Cc3cc(C(F)(F)F)cc(C4(C(C)C)C(C#N)=C(N)Oc5[nH]nc(C)c54)c3)CC2)o1] | | | | |

Compounds of structural formula (I) can be made according to the methods described herein. Alternative methods of making compounds of structural formula (I) are within the abilities of a person skilled in the art.

### Compositions

Also provided herein is a composition (e.g., pharmaceutical composition), comprising a compound disclosed herein (e.g., a compound of any one of the structural formulas depicted herein), or a pharmaceutically acceptable salt thereof, and a pharmaceutically acceptable carrier. The compositions can be used in the methods described herein, e.g., to supply a compound described herein, or a pharmaceutically acceptable salt thereof.

Compositions described herein and, hence, compounds of the disclosure can be administered orally, parenterally (including subcutaneously, intramuscularly, intravenously and intradermally), by inhalation spray, topically, rectally, nasally, buccally, vaginally or via an implanted reservoir. In some embodiments, provided compounds or compositions are administrable intravenously and/or intraperitoneally. The term “parenteral,” as used herein, includes subcutaneous, intracutaneous, intravenous, intramuscular, intraocular, intravitreal, intra-articular, intra-arterial, intra-synovial, intrasternal, intrathecal, intralesional, intrahepatic, intraperitoneal intralesional and intracranial injection or infusion techniques. Preferably, the compositions are administered orally, subcutaneously, intraperitoneally or intravenously (e.g., orally).

Compositions provided herein can be orally administered in any orally acceptable dosage form including, but not limited to, capsules, tablets, aqueous suspensions, dispersions and solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions and/or emulsions are required for oral use, the active ingredient can be suspended or dissolved in an oily phase and combined with emulsifying and/or suspending agents. If desired, certain sweetening, flavoring or coloring agents may also be added.

In some embodiments, an oral formulation is formulated for immediate release or sustained/delayed release.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders, such as carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium salts, (g) wetting agents, such as acetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Liquid dosage forms for oral administration include pharmaceutically acceptable emulsions, microemulsions, solutions, suspensions, syrups, and elixirs. In addition to the compound of the present disclosure, the liquid dosage forms may contain inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, or mixtures thereof. Besides inert diluents, the oral compositions can also include adjuvants such as wetting agents, emulsifying and suspending agents, sweetening, flavoring, coloring, perfuming, and preservative agents.

Compositions suitable for buccal or sublingual administration include tablets, lozenges and pastilles, wherein the active ingredient is formulated with a carrier such as sugar and acacia, tragacanth, or gelatin and glycerin.

Solid compositions of a similar type may also be employed as fillers in soft and hard-filled gelatin capsules using excipients such as lactose or milk sugar, as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

A compound of the disclosure can also be in micro-encapsulated form with one or more excipients, as noted above. In such solid dosage forms, the compound can be admixed with at least one inert diluent such as sucrose, lactose or starch. Such dosage forms can also comprise, as is normal practice, additional substances other than inert diluents, e.g., tableting lubricants and other tableting aids such a magnesium stearate and microcrystalline cellulose.

Compositions for oral administration may be designed to protect the active ingredient against degradation as it passes through the alimentary tract, for example, by an outer coating of the formulation on a tablet or capsule.

In another embodiment, a compound of the disclosure can be provided in an extended (or “delayed” or “sustained”) release composition. This delayed-release composition comprises the compound or pharmaceutically acceptable salt in combination with a delayed-release component. Such a composition allows targeted release of a provided agent into the lower gastrointestinal tract, for example, into the small intestine, the large intestine, the colon and/or the rectum. In certain embodiments, a delayed-release composition further comprises an enteric or pH-dependent coating, such as cellulose acetate phthalates and other phthalates (e.g., polyvinyl acetate phthalate, methacrylates (Eudragits)). Alternatively, the delayed-release composition provides controlled release to the small intestine and/or colon by the provision of pH sensitive methacrylate coatings, pH sensitive polymeric microspheres, or polymers which undergo degradation by hydrolysis. The delayed-release composition can be formulated with hydrophobic or gelling excipients or coatings. Colonic delivery can further be provided by coatings which are digested by bacterial enzymes such as amylose or pectin, by pH dependent polymers, by hydrogel plugs swelling with time (Pulsincap), by time-dependent hydrogel coatings and/or by acrylic acid linked to azoaromatic bonds coatings.

Compositions described herein can also be administered subcutaneously, intraperitoneally or intravenously. Compositions described herein for intravenous, subcutaneous, or intraperitoneal injection may contain an isotonic vehicle such as sodium chloride injection, Ringer's injection, dextrose injection, dextrose and sodium chloride injection, lactated Ringer's injection, or other vehicles known in the art.

Compositions described herein can also be administered in the form of suppositories for rectal administration. These can be prepared by mixing a compound of the disclosure with a suitable non-irritating excipient that is solid at room temperature but liquid at rectal temperature and, therefore, will melt in the rectum to release the drug. Such materials include cocoa butter, beeswax and polyethylene glycols.

Compositions described herein can also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches can also be used.

For other topical applications, the compositions can be formulated in a suitable ointment containing the active component suspended or dissolved in one or more carriers. Carriers for topical administration of a compound of the disclosure include, but are not limited to, mineral oil, liquid petrolatum, white petrolatum, propylene glycol, polyoxyethylene, polyoxypropylene compound, emulsifying wax and water and penetration enhancers. Alternatively, compositions can be formulated in a suitable lotion or cream containing the active compound suspended or dissolved in one or more pharmaceutically acceptable carriers. Alternatively, the composition can be formulated with a suitable lotion or cream containing the active compound suspended or dissolved in a carrier with suitable emulsifying agents. In some embodiments, suitable carriers include, but are not limited to, mineral oil, sorbitan monostearate, polysorbate 60, cetyl esters wax, cetearyl alcohol, 2-octyldodecanol, benzyl alcohol and water. In other embodiments, suitable carriers include, but are not limited to, mineral oil, sorbitan monostearate, polysorbate 60, cetyl esters wax, cetearyl alcohol, 2-octyldodecanol, benzyl alcohol and water and penetration enhancers.

For ophthalmic use, compositions can be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic use, the compositions can be formulated in an ointment such as petrolatum.

Compositions can also be administered by nasal aerosol or inhalation, for example, for the treatment of asthma. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents. Without wishing to be bound by any particular theory, it is believed that local delivery of a composition described herein, as can be achieved by nasal aerosol or inhalation, for example, can reduce the risk of systemic consequences of the composition, for example, consequences for red blood cells.

Other pharmaceutically acceptable carriers, adjuvants and vehicles that can be used in the compositions described herein include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives can also be advantageously used to enhance delivery of agents described herein.

The compositions can be in the form of a sterile injectable preparation, for example, as a sterile injectable aqueous or oleaginous suspension. This suspension can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium. For this purpose, any bland fixed oil can be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms can also be used for the purposes of formulation.

In some embodiments, compositions comprising a compound of the disclosure can also include one or more additional therapeutic agents, e.g., for use in combination with a compound of the disclosure as, for example, when the compound of the disclosure and the one or more additional therapeutic agents are to be co-administered.

Some embodiments provide a combination (e.g., pharmaceutical combination) comprising a compound of the disclosure (e.g., a composition described herein comprising a compound of the disclosure) and one or more additional therapeutic agents (e.g., one or more compositions comprising one or more additional therapeutic agents). Such combinations are particularly useful as, for example, when the compound of the disclosure and the one or more additional therapeutic agents are to be administered separately. In a combination provided herein, the compound of the disclosure and the one or more additional therapeutic agents can be administrable by the same route of administration or by different routes of administration.

Also provided herein is a kit comprising a compound of the disclosure and an additional therapeutic agent(s) (e.g., an additional therapeutic agent described herein). In one embodiment, the kit comprises an effective amount of a compound of the disclosure to treat a disease, disorder or condition described herein, and an effective amount of an additional therapeutic agent(s) to treat the disease, disorder or condition. In some embodiments, the kit further comprises written instructions for administering the compound of the disclosure and the additional agent(s) to a subject to treat a disease, disorder or condition described herein.

In certain embodiments, the one or more additional therapeutic agents comprise a rescue therapy. “Rescue therapy,” as used herein, refers to a therapeutic agent intended to reduce toxicity of another agent (e.g., a compound of the disclosure) with which it is being combined. A rescue therapy can reduce toxicity of another agent by interfering with the agent's ability to produce a toxic side effect, as formate typically does, for example. Alternatively, a rescue therapy can reduce toxicity of another agent by treating the toxic side effect produced by the agent, as an anti-nausea agent or other palliative care agents do, for example. Examples of rescue therapies include formate, or a derivative thereof, such as a formate ester, glycine, leucovorin, or a pharmaceutically acceptable salt of any of the foregoing. In some embodiments, the rescue therapy is formate, or a derivative thereof, such as a formate ester, glycine, leucovorin, or a pharmaceutically acceptable salt of any of the foregoing, or a combination of two or more of any of the foregoing.

Formate has been shown to rescue the effects of SHMT inhibitors in many cell types. In B-cell lymphomas, such as diffuse large B-cell lymphoma, formate has been observed to increase the inhibitory effect of SHMT inhibitors with which it was combined. Thus, in some embodiments, the additional therapeutic agent is formate, or a derivative thereof, such as a formate ester, or a pharmaceutically acceptable salt of any of the foregoing. In B-cell lymphomas, where formate was observed not to rescue the effects of SHMT inhibitors, a combination of formate and glycine has been shown to rescue the effects of SHMT inhibitors.

In other embodiments, the one or more additional therapeutic agents comprise an additional anti-cancer agent (e.g., a chemotherapeutic agent, an anti-folate, an immunotherapy). Thus, in some embodiments, the one or more additional therapeutic agents comprise an anti-folate. Anti-folates are agents that antagonize the action of folic acid. Traditionally, antifolate therapies function mainly through inhibition of the cytosolic folate enzymes dihydrofolate reductase (DHFR) and thymidylate synthetase (TS), resulting in impaired DNA synthesis and impaired cellular replication. Examples of anti-folates include methotrexate and pemetrexed, or a pharmaceutically acceptable salt of the foregoing.

In some embodiments, the one or more additional therapeutic agents comprise a chemotherapeutic agent. Examples of chemotherapeutic agents include alkylating agents, such as thiotepa and cyclophosphamide (CYTOXAN™); alkyl sulfonates, such as busulfan, improsulfan and piposulfan; aziridines, such as benzodopa, carboquone, meturedopa, and uredopa; ethylenimines and methylamelamines, such as altretamine, triethylenemelamine, trietylenephosphoramide, triethylenethiophosphaoramide and trimethylolomelamine; acetogenins, such as bullatacin and bullatacinone; camptothecins, including the synthetic analogue topotecan; bryostatin; callystatin; CC-1065, including its adozelesin, carzelesin and bizelesin analogues; cryptophycins, such as cryptophycin 1 and cryptophycin 8; dolastatin; duocarmycin, including the synthetic analogues, KW-2189 and CBI-TMI; eleutherobin; pancratistatin; sarcodictyins; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosoureas, such as carmustine, chlorozotocin, fotemustine, lomustine, nimustine, and ranimustine; antibiotics, such as the enediyne antibiotics (e.g., calicheamicin, especially calicheamicin gamma 1 and calicheamicin theta I, see, e.g., Angew Chem. Intl. Ed. Engl. 33:183-186 (1994); dynemicin, such as dynemicin A; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, methotrexate, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, floxuridine, and 5-FU; androgens, such as calusterone, dromostanolone propionate, epitiostanol, mepitiostane, and testolactone; anti-adrenals, such as aminoglutethimide, mitotane, and trilostane; folic acid replenishers, such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; epothilones; etoglucid; gallium nitrate; hydroxyurea; lentinan; lonidamine; maytansinoids, such as maytansine and ansamitocins; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofuran; spirogermanium; tenuazonic acid; triaziquone; 2,2′,2″-trichlorotriethylamine; trichothecenes, such as T-2 toxin, verracurin A, roridin A and anguidine; urethan; vindesine; dacarbazine; mannomustine; mitobronitol; mitolactol; pipobroman; gacytosine; arabinoside (“Ara-C”); cyclophosphamide; thiotepa; taxoids, such as paclitaxel (e.g., TAXOL™, Bristol-Myers Squibb Oncology, Princeton, N.J.; Nab-paclitaxel, such as the nanoparticle albumin-bound paclitaxel sold as ABRAXANE®) and doxetaxel (TAXOTERE®, Rhone-Poulenc Rorer, Antony, France); chlorambucil; gemcitabine; 6-thioguanine; mercaptopurine; methotrexate; folinic acid; platinum analogs such as cisplatin, oxaliplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitors, such as irinotecan and RFS 2000; difluoromethylomithine (DFMO); retinoic acid; and capecitabine; or a pharmaceutically acceptable salt, acid or derivative of any of the above. Further examples of chemotherapeutic agents include anti-hormonal agents that act to regulate or inhibit hormone action on tumors, such as anti-estrogens including, for example, tamoxifen, raloxifene, aromatase inhibiting 4(5)-imidazoles, 4-hydroxytamoxifen, trioxifene, keoxifene, LY117018, onapristone, and toremifene (Fareston); anti-androgens, such as flutamide, nilutamide, bicalutamide, leuprolide, and goserelin; and siRNA, or a pharmaceutically acceptable salt, acid or derivative of any of the above.

In some embodiments, the one or more additional therapeutic agents comprise an immunotherapy. Examples of immunotherapies include immune checkpoint inhibitors, agonist CD-40 antibodies, T-cell transfer therapy (e.g., CAR T-cell therapy, tumor infiltrating lymphocytes (TIL) therapy), NK cell transfer therapy (e.g., CAR NK-cell therapy), monoclonal antibodies, cancer treatment vaccines and immune system modulators. Examples of immune checkpoint inhibitors include inhibitors of CTLA-4, such as ipilimumab; inhibitors of PD-1, such as pembrolizumab, nivolumab, and cemiplimab; and inhibitors of PD-L1, such as atezolizumab, avelumab, and durvalumab. Examples of T-cell transfer therapies include tisagenlecleucel (KYMRIAH™) and axicabtagene ciloleucel (YESCARTA™). Examples of monoclonal antibodies include rituximab and blinatumomab. Examples of cancer treatment vaccines include talimogene laherparepvec (T-VEC, or IMLYGIC®). Examples of immune system modulators include cytokines, such as interleukins (e.g., IL-2) and interferons (e.g., INF-alpha); hematopoietic growth factors, such as erythropoietin, IL-11, granulocyte-macrophage colony-stimulating factor, and granulocyte colony-stimulating factor; Bacillus Calmette-Guérin (BCG); and immunomodulatory agents, such as thalidomide, lenalidomide, pomalidomide, and imiquimod.

The compositions described herein can be provided in unit dosage form. The amount of a compound of the disclosure that can be combined with the carrier materials to produce a composition in a single dosage form will vary depending, for example, upon the host treated, the particular mode of administration and the activity of the agent employed. Preferably, compositions should be formulated so that a dosage of from about 0.01 mg/kg to about 100 mg/kg body weight/day of the compound of the disclosure can be administered to a subject receiving the composition. In some embodiments, compositions are formulated so that a dosage described herein of a compound of the disclosure can be administered to a subject receiving the composition. A unit dosage form may contain from about 1 mg to about 5,000 mg, from about 10 mg to about 2,500 mg, from about 100 mg to about 1,000 mg, from about 1 mg to about 1000 mg, from about 1 mg to about 500 mg, from about 1 mg to about 250 mg, from about 1 mg to about 150 mg, from about 0.5 mg to about 100 mg, or from about 1 mg to about 50 mg of active ingredient(s).

The desired dose may conveniently be administered in a single dose, for example, such that the agent is administered once per day, or as multiple doses administered at appropriate intervals, for example, such that the agent is administered 2, 3, 4, 5, 6 or more times per day. The daily dose can be divided, especially when relatively large amounts are administered, or as deemed appropriate, into several, for example 2, 3, 4, 5, 6 or more, administrations. Typically, the compositions will be administered from about 1 to about 6 (e.g., 1, 2, 3, 4, 5 or 6) times per day or, alternatively, as an infusion (e.g., a continuous infusion).

The compositions described herein can, for example, be administered by injection, intravenously, intraarterially, intraocularly, intravitreally, subdermally, orally, buccally, nasally, transmucosally, topically, in an ophthalmic preparation, or by inhalation, with a dosage ranging from about 0.5 mg/kg to about 100 mg/kg of body weight or, alternatively, in a dosage ranging from about 1 mg/dose to about 1000 mg/dose, every 4 to 120 hours, or according to the requirements of the particular drug. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Determining the dosage for a particular agent, subject and disease is well within the abilities of one of skill in the art. Preferably, the dosage does not cause or produces minimal adverse side effects.

Doses lower or higher than those recited above may be required. Specific dosage and treatment regimens for any particular patient will depend upon a variety of factors, for example, the activity of the specific agent employed, the age, body weight, general health status, sex, diet, time of administration, rate of excretion, drug combination, the severity and course of the disease, condition or symptoms, the patient's disposition to the disease, condition or symptoms, the judgment of the treating physician and the severity of the particular disease being treated. The amount of an agent in a composition will also depend upon the particular agent in the composition.

A typical composition described herein will contain from about 1% to about 95%, from about 2.5% to about 95% or from about 5% to about 95% active compound (w/w). Alternatively, a preparation can contain from about 20% to about 80% active compound (w/w).

In some embodiments, the concentration of one or more therapeutic agents provided in a composition is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4% 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v; and/or greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50% 3.25%, 3%, 2.75%, 2.50%, 2.25%, 2%, 1.75%, 1.50%, 125%, 1%, 0.5%, 0.4%, 0.3%, 0.2%, 0.1%, 0.09% 0.08% 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more therapeutic agents provided in a composition is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, about 1% to about 10% w/w, w/v or v/v. In some embodiments, the concentration of one or more therapeutic agents provided in a pharmaceutical composition is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.10% to about 0.9% w/w, w/v or v/v.

### Uses

Serine catabolism is initiated by serine hydroxymethyltransferase (SHMT) activity, catalyzed in the cytosol by SHMT1 and in the mitochondria by SHMT2. SHMTs catalyze a reversible reaction converting serine to glycine, with concurrent methylene-tetrahydrofolate (THF) generation. Increased SHMT enzyme activity has been detected in human breast cancer, colon cancer, and in rat sarcoma.

SHMT functions to generate one-carbon units for cellular folate metabolism. Inhibition of other aspects of folate metabolism is an established mechanism of therapy for a variety of cancers and autoimmune diseases. However, existing anti-folates are characterized by dose-limiting toxicity that limits their effectiveness in cancer therapy and their tolerability in autoimmune disease.

Hypoxia occurs in the tumor environment, and the mitochondrial form of SHMT, SHMT2, is induced under hypoxic stress. SHMT expression may help tumor cells survive under hypoxic conditions, thus promoting cancerous cell growth, survival and metastasis. Hypoxic cells are generally more resistant to radiation and chemotherapy treatment, further permitting the tumor to grow and metastasize. SHMT2 overexpression has been observed in various different cancers, including neuroblastoma, bladder cancer, colorectal cancer, kidney cancer, etc.

Compounds of the disclosure exhibit effects consistent with inhibition of SHMT (e.g., SHMT1, SHMT2 or SHMT1 and SHMT2). Accordingly, provided herein is a method of modulating (e.g., inhibiting) a SHMT (e.g., SHMT1, SHMT2 or SHMT1 and SHMT2), comprising contacting a cell with a compound of the disclosure. In some embodiments, the cell is in a subject (e.g., a human). In some embodiments, the method is for treating a disease, condition or disorder described herein (e.g., a disease, disorder or condition associated with SHMT activity; cancer; autoimmune disease; fibrosis).

Also provided herein is a method of treating a disease, disorder or condition associated with SHMT (e.g., SHMT1, SHMT2 or SHMT1 and SHMT2) activity or expression in a subject in need thereof, comprising administering to the subject a therapeutically effective amount of a compound of the disclosure or a composition described herein. As used herein, “a disease, disorder or condition associated with SHMT activity or expression” refers to any disease, disorder or condition that is directly or indirectly regulated by SHMT. Examples of diseases, disorders or conditions associated with SHMT activity include, but are not limited to, cancer, autoimmune diseases and fibrosis, as well as others described herein. In some embodiments, the disease, disorder or condition associated with SHMT activity or expression is associated with elevated SHMT activity or expression.

Also provided herein is a method of treating a disease, disorder or condition associated with altered mitochondrial metabolism, such as mitochondrial folate metabolism, in a subject in need thereof, comprising administering to the subject a therapeutically effective amount of a compound of the disclosure or a composition described herein. In some embodiments, the disease, disorder or condition associated with altered mitochondrial metabolism is associated with increased mitochondrial metabolism, such as increased mitochondrial folate metabolism. Genes or proteins involved in mitochondrial metabolism that can be dysregulated in, for example, cancer cells, include, for example, MTHFD1, MTHFD1L, MTHFD2, MTHFD2L, SHMT1, SHMT2, MTHFR, ALDH1L1, ALDHL2, SLC25A32 (also known as the mitochondrial folate transporter MFT), FH, KEAP1, and NRF2.

In certain embodiments, the disease, disorder or condition is a disease, disorder or condition (e.g., a malignancy of B-cell origin, such as a cancer of B-cell origin) in which the SLC38A2 gene is dysregulated (e.g., down-regulated). Diseases, disorders or conditions in which the SLC38A2 gene is dysregulated include lymphoma (e.g., B-cell lymphoma, such as diffuse large B-cell lymphoma). Moreover, it has been observed that decreased expression of SLC38A2, as assessed by mRNA expression, is correlated with sensitivity to a combination of an SHMT inhibitor and a rescue therapy (e.g., formate), wherein the rescue therapy increases the inhibitory effect of SHMT inhibitors with which it is combined.

Also provided herein is a method of treating a cancer in a subject in need thereof, comprising administering to the subject a therapeutically effective amount of a compound of the disclosure or a composition described herein.

Although the high metabolic needs of cancer cells make all cancers good candidates for treatment with SHMT inhibitors, certain cancers may be particularly susceptible to treatment or may be sensitized to treatment due to their underlying mitochondrial activity or mutational status. By way of non-limiting example, in certain embodiments, the cancer comprises cells having a mutation affecting mitochondrial metabolism or the mitochondrial folate pathway (e.g., resulting in elevated expression levels of SHMT2, or upregulation in a component of the mitochondrial folate pathway). Other mutations affecting mitochondrial metabolism or the mitochondrial folate pathway may impair mitochondrial metabolism or the mitochondrial folate pathway and, thus, sensitize cancers to SHMT inhibition. Examples of genes or proteins that may be dysregulated in, for example, cancer cells, include, for example, MTHFD1, MTHFD1L, MTHFD2, MTHFD2L, SHMT1, SHMT2, MTHFR, ALDH1L1, ALDHL2, SLC25A32 (also known as the mitochondrial folate transporter MFT), FH, KEAP1, and NRF2.

Other classes of cancers that may be particularly susceptible to treatment with an SHMT inhibitor are cancers comprising mutation(s) that inactivate KEAP1 (for example, by somatic mutation or epigenetic silencing). Such mutations may result in aberrant NRF2 activity and nuclear function. Additionally or alternatively, the cancer may additionally have mutation(s) in NRF2 itself with or without KEAP1 mutation(s). Cancers may additionally or alternatively have alteration(s) in mitochondrial metabolism, including mutation(s) to fumarate hydratase (FH) that lead to activation of NRF2. Loss of FH activity may potentiate cells to SHMT inhibitors by additional mechanisms as well. Aberrant NRF2 activation leads to altered transcription of mitochondrial and one carbon metabolism genes through the activity of ATF4. Activation of the 1C pathway and mitochondrial folate metabolism by NRF2 may also occur via ATF4-independent mechanisms. Examples of cancers with identified mutations in the KEAP1-NRF2-ATF4 pathway include non-small cell lung cancer, squamous cell lung carcinoma, prostate cancer, head and neck cancer (KEAP1 mutations, NRF2 mutations), hereditary papillary renal carcinoma, hereditary leiomyomatosis and renal cell cancer (FH mutations). As described herein, there are identified cancers with elevated SHMT2 levels and/or mutations in other folate pathway components as well.

Thus, a wide variety of cancers is treatable according to the methods described herein. In some embodiments, the cancer comprises a solid tumor (e.g., a tumor of the breast, lung, prostate, colorectal, bladder, ovary, uterus kidney, stomach, colon, rectum, testes, head and/or neck, pancreas, brain, skin, trophoblastic neoplasm). Accordingly, in some embodiments, the cancer is a solid tumor cancer (e.g., breast, lung, such as non-small cell lung, prostate, colorectal, bladder, ovarian, uterine, kidney, stomach, colon, rectum, testes, head and/or neck, pancreatic, brain, skin cancer; trophoblastic neoplasia; mesothelioma; glioblastoma). In some embodiments, the cancer is a hematologic cancer (e.g., leukemia, lymphoma, myeloma). Hematologic cancers that can be treated according to the methods described herein include leukemias (e.g., acute leukemias, chronic leukemias), lymphomas (e.g., B-cell lymphoma, T-cell lymphoma, NK cell lymphoma) and multiple myeloma. Examples of leukemias include acute lymphoblastic leukemia (ALL), acute myelogenous leukemia (AML), chronic lymphocytic leukemia (CLL), chronic myelogenous leukemia (CML) and acute monocytic leukemia (AMoL). Examples of lymphomas include Hodgkin's lymphoma and non-Hodgkin's lymphoma.

Examples of cancers treatable according to the methods described herein include Acute Lymphoblastic Leukemia (ALL); Acute Myeloid Leukemia (AML); Adrenocortical Carcinoma; Adrenocortical Carcinoma, Childhood; AIDS-Related Cancer (e.g., Kaposi Sarcoma, AIDS-Related Lymphoma, Primary CNS Lymphoma); Anal Cancer; Appendix Cancer; Astrocytomas, Childhood; Atypical Teratoid/Rhabdoid Tumor, Childhood, Central Nervous System; Basal Cell Carcinoma of the Skin; Bile Duct Cancer; Bladder Cancer; Bladder Cancer, Childhood; Bone Cancer (including Ewing Sarcoma, Osteosarcoma and Malignant Fibrous Histiocytoma); Brain Tumors/Cancer; Breast Cancer; Burkitt Lymphoma; Carcinoid Tumor (Gastrointestinal); Carcinoid Tumor, Childhood; Cardiac (Heart) Tumors, Childhood; Embryonal Tumors, Childhood; Germ Cell Tumor, Childhood; Primary CNS Lymphoma; Cervical Cancer; Childhood Cervical Cancer; Cholangiocarcinoma; Chordoma, Childhood; Chronic Lymphocytic Leukemia (CLL); Chronic Myelogenous Leukemia (CML); Chronic Myeloproliferative Neoplasms; Colorectal Cancer; Childhood Colorectal Cancer; Craniopharyngioma, Childhood; Cutaneous T-Cell Lymphoma (e.g., Mycosis Fungoides and Sezary Syndrome); Ductal Carcinoma In Situ (DCIS); Embryonal Tumors, Central Nervous System, Childhood; Endometrial Cancer (Uterine Cancer); Ependymoma, Childhood; Esophageal Cancer; Childhood Esophageal Cancer; Esthesioneuroblastoma; Ewing Sarcoma; Extracranial Germ Cell Tumor, Childhood; Extragonadal Germ Cell Tumor; Eye Cancer; Childhood Intraocular Melanoma; Intraocular Melanoma; Retinoblastoma; Fallopian Tube Cancer; Fibrous Histiocytoma of Bone, Malignant, and Osteosarcoma; Gallbladder Cancer; Gastric (Stomach) Cancer; Childhood Gastric (Stomach) Cancer; Gastrointestinal Carcinoid Tumor; Gastrointestinal Stromal Tumors (GIST); Childhood Gastrointestinal Stromal Tumors; Germ Cell Tumors; Childhood Central Nervous System Germ Cell Tumors (e.g., Childhood Extracranial Germ Cell Tumors, Extragonadal Germ Cell Tumors, Ovarian Germ Cell Tumors, Testicular Cancer); Gestational Trophoblastic Disease; Hairy Cell Leukemia; Head and Neck Cancer; Heart Tumors, Childhood; Hepatocellular (Liver) Cancer; Histiocytosis, Langerhans Cell; Hodgkin Lymphoma; Hypopharyngeal Cancer; Intraocular Melanoma; Childhood Intraocular Melanoma; Islet Cell Tumors, Pancreatic Neuroendocrine Tumors; Kaposi Sarcoma; Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Childhood Lung Cancer; Lymphoma; Male Breast Cancer; Malignant Fibrous Histiocytoma of Bone and Osteosarcoma; Melanoma; Childhood Melanoma; Melanoma, Intraocular (Eye); Childhood Intraocular Melanoma; Merkel Cell Carcinoma; Mesothelioma, Malignant; Childhood Mesothelioma; Metastatic Cancer; Metastatic Squamous Neck Cancer with Occult Primary; Midline Tract Carcinoma With NUT Gene Changes; Mouth Cancer; Multiple Endocrine Neoplasia Syndromes; Multiple Myeloma/Plasma Cell Neoplasms; Mycosis Fungoides; Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms, Chronic; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip and Oral Cavity Cancer and Oropharyngeal Cancer; Osteosarcoma and Malignant Fibrous Histiocytoma of Bone; Ovarian Cancer; Childhood Ovarian Cancer; Pancreatic Cancer; Childhood Pancreatic Cancer; Pancreatic Neuroendocrine Tumors; Papillomatosis (Childhood Laryngeal); Paraganglioma; Childhood Paraganglioma; Paranasal Sinus and Nasal Cavity Cancer; Parathyroid Cancer; Penile Cancer; Pharyngeal Cancer; Pheochromocytoma; Childhood Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer; Renal Cell (Kidney) Cancer; Retinoblastoma; Rhabdomyosarcoma, Childhood; Salivary Gland Cancer; Sarcoma (e.g., Childhood Rhabdomyosarcoma, Childhood Vascular Tumors, Ewing Sarcoma, Kaposi Sarcoma, Osteosarcoma (Bone Cancer), Soft Tissue Sarcoma, Uterine Sarcoma); Sezary Syndrome; Skin Cancer; Childhood Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; Childhood Stomach (Gastric) Cancer; T-Cell Lymphoma, Cutaneous (e.g., Mycosis Fungoides and Sezary Syndrome); Testicular Cancer; Childhood Testicular Cancer; Throat Cancer (e.g., Nasopharyngeal Cancer, Oropharyngeal Cancer, Hypopharyngeal Cancer); Thymoma and Thymic Carcinoma; Thyroid Cancer; Transitional Cell Cancer of the Renal Pelvis and Ureter; Ureter and Renal Pelvis, Transitional Cell Cancer; Urethral Cancer; Uterine Cancer, Endometrial; Uterine Sarcoma; Vaginal Cancer; Childhood Vaginal Cancer; Vascular Tumors; Vulvar Cancer; and Wilms Tumor and Other Childhood Kidney Tumors.

Metastases of the aforementioned cancers can also be treated in accordance with the methods described herein. In some embodiments, the cancer is a metastatic cancer.

In some embodiments, the cancer is an adult cancer. In some embodiments, the cancer is a pediatric cancer. In some embodiments, the cancer is an adult and pediatric cancer.

In some embodiments, the cancer is a B-cell lymphoma, such as diffuse large B-cell lymphoma or Burkitt's lymphoma. In certain embodiments, the B-cell lymphoma is characterized by the following in vitro activity: growth sensitivity to an SHMT inhibitor in vitro that is not rescued by formate but is rescued by a combination of formate and glycine, wherein growth sensitivity is assessed by cell count in vitro. In some embodiments, the glycine rescue is sufficient to restore glycine levels in the normal body (e.g., a healthy cell or tissue) but not in the lymphoma.

Metabolic flux through SHMT1 and/or SHMT2 is required for the synthesis of purines and thymidine in activated immune cells, such as T cells, as in autoimmune disease, to enable their proliferation. Thus, also provided herein is a method of treating an autoimmune disease, comprising administering to the subject a therapeutically effective amount of a compound of the disclosure or a composition described herein.

Autoimmune diseases include, but are not limited to, rheumatoid arthritis, multiple sclerosis, systemic lupus erythematosus, type 1 diabetes mellitus, Guillain-Barre syndrome, chronic inflammatory demyelinating polyneuropathy, Graves' disease, Hashimoto's thyroiditis, myasthenia gravis, inflammatory bowel disease (e.g., Crohn's disease or ulcerative colitis), polymyositis, dermatomyositis, inflammatory myositis, ankylosing spondolytis, psoriasis, vasculitis, sarcoidosis, eczema, vasculitis, Sjogren's disease and transplant rejection.

Fibrosis is caused by excess deposition of extracellular matrix. Extracellular matrix comprises collagen, whose amino acid composition is enriched in the amino acid glycine, a direct product of the reaction catalyzed by SHMT. Expression of SHMT2 is upregulated in idiopathic pulmonary fibrosis. Nigdelioglu, R., et al., J. Biol. Chem. 2016; 291(53): 27239-27251, the entire contents of which is incorporated herein by reference in its entirety. Without being bound by any particular theory, it is also thought that SHMT inhibition may suppress inflammatory events that cause or exacerbate fibrosis, or may otherwise rewire the metabolism of fibroblasts, including suppressing fibroblast collagen synthesis, other extracellular matrix synthesis and/or proliferation, and/or may mitigate the downstream consequences of fibrotic disease, including associated proliferative disorders such as hepatocellular carcinoma in the case of liver fibrosis. Thus, also provided herein is a method of treating fibrosis and/or a fibrotic disease, comprising administering to the subject a therapeutically effective amount of a SHMT inhibitor (e.g., a SHMT1 and/or SHMT2 inhibitor). In some embodiments, the SHMT inhibitor is a compound of the disclosure or a composition described herein. Additional SHMT inhibitors are described in U.S. Patent Application Publication Nos. US 2018/0117010 and US 2018/0072751, the entire contents of which are incorporated herein by reference. In some embodiments, the SHMT inhibitor is a compound described in U.S. Patent Application Publication Nos. US 2018/0117010 or US 2018/0072751, or a pharmaceutically acceptable salt thereof, or a composition described therein.

Fibroses and fibrotic diseases include, but are not limited to, systemic sclerosis, scleroderma, pulmonary fibrosis, idiopathic pulmonary fibrosis, primary biliary cholangitis, liver fibrosis, cirrhosis, fatty liver disease, non-alcoholic fatty liver disease, chronic hepatitis B or C, heart fibrosis, post-myocardial infarction cardiac changes, interstitial fibrosis, replacement fibrosis, mediastinal fibrosis, bone marrow fibrosis, myelofibrosis, pancreas fibrosis, skin fibrosis, nephrogenic systemic fibrosis, keloid formation, renal fibrosis, Peyronie's disease, contractures, arthrofibrosis, Crohn's disease, adhesive capsulitis, excessive surgical scarring and retroperitoneal cavity fibrosis. In some embodiments, the fibrosis is organ fibrosis.

A compound of the disclosure or a composition described herein can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending, for example, on the compound, salt or composition and the particular disease to be treated. Administration can be local or systemic as indicated. The preferred mode of administration can vary depending on the particular compound chosen. In some embodiments, administration is intravenous, subcutaneous or oral (e.g., oral).

A compound of the disclosure or a composition described herein can also be administered in combination with one or more other therapies (e.g., radiation therapy; a chemotherapy, such as a chemotherapeutic agent; an immunotherapy; rescue therapy). When administered in a combination therapy, the compound of the disclosure can be administered before, after or concurrently with the other therapy (e.g., radiation therapy, an additional agent(s)). When co-administered simultaneously (e.g., concurrently), the compound of the disclosure and other therapy can be in separate formulations or the same formulation. Alternatively, the compound of the disclosure and other therapy can be administered sequentially, as separate compositions, within an appropriate time frame as determined by a skilled clinician (e.g., a time sufficient to allow an overlap of the pharmaceutical effects of the therapies). When the compound of the disclosure and the other therapy (e.g., therapeutic agent) are administered as separate formulations or compositions, the compound of the disclosure and the other therapy can be administered by the same route of administration or by different routes of administration, including any of the routes of administration described herein.

In some embodiments, a method described herein further comprises administering to the subject a therapeutically effective amount of an additional therapy (e.g., radiation therapy, rescue therapy, anti-cancer therapy). In some embodiments, a method described herein further comprises administering to the subject a therapeutically effective amount of one or more additional therapeutic agents (e.g., any of the additional therapeutic agents described herein, for example, with respect to the compositions described herein).

The compounds and compositions described herein (e.g., a compound of the disclosure) may be administered in a dosage of from about 0.01 mg/kg to about 100 mg/kg body weight/day of active ingredient(s), or in a dosage of from about 1 mg to about 5,000 mg, e.g., from about 10 mg to about 2,500 mg, from about 100 mg to about 1,000 mg, from about 1 mg to about 1000 mg, from about 1 mg to about 500 mg, from about 1 mg to about 250 mg, from about 1 mg to about 150 mg, from about 0.5 mg to about 100 mg, or from about 1 mg to about 50 mg of active ingredient(s). Suitable dosages also include from about 0.001 mg/kg to about 100 mg/kg, e.g., from about 0.01 mg/kg to about 100 mg/kg, from about 0.01 mg/kg to about 10 mg/kg, or from about 0.01 mg/kg to about 1 mg/kg body weight per treatment.

The desired dose may conveniently be administered in a single dose, for example, such that the agent is administered once per day (e.g., QD), or as multiple doses administered at appropriate intervals, for example, such that the agent is administered 2, 3, 4, 5, 6 or more times per day (e.g., BID, TID, QID). The daily dose can be divided, especially when relatively large amounts are administered, or as deemed appropriate, into several, for example 2, 3, 4, 5, 6 or more, administrations. Typically, the compositions will be administered from about 1 to about 6 (e.g., 1 or 2, 1, 2, 3, 4, 5 or 6) times per day or, alternatively, as an infusion (e.g., a continuous infusion).

The compounds and compositions described herein (e.g., a compound of the disclosure) can, for example, be administered by injection, intravenously, intraarterially, intraocularly, intravitreally, subdermally, orally, buccally, nasally, transmucosally, topically, in an ophthalmic preparation, or by inhalation, with a dosage ranging from about 0.5 mg/kg to about 100 mg/kg of body weight or, alternatively, in a dosage ranging from about 1 mg/dose to about 1000 mg/dose, every 4 to 120 hours, or according to the requirements of the particular drug. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Determining the dosage for a particular agent, subject and disease is well within the abilities of one of skill in the art. Preferably, the dosage does not cause or produces minimal adverse side effects.

Doses lower or higher than those recited above may be required. Specific dosage and treatment regimens for any particular patient will depend upon a variety of factors, for example, the activity of the specific agent employed, the age, body weight, general health status, sex, diet, time of administration, rate of excretion, drug combination, the severity and course of the disease, condition or symptoms, the patient's disposition to the disease, condition or symptoms, the judgment of the treating physician and the severity of the particular disease being treated. The amount of an agent in a composition will also depend upon the particular agent in the composition.

In addition to being therapeutic agents, the compounds of the disclosure may be useful as research tools, e.g., as a tool compound for altering SHMT activity in cells; studying SHMT function and/or serine flux and/or folate metabolism and/or NADPH generation and/or glycine generation in, for example, healthy cells, cancerous cells, hypoxic cells and/or mutant cells, such as cells in which the activity of MTHFD1, MTHFD1L, MTHFD2, MTHFD2L, SHMT1, SHMT2, MTHFR, ALDHIL1, ALDHL2, FH and/or KEAP1 is or has been modulated (e.g., inhibited), dysregulated or knocked out, or cells having certain hyperactivating mutations in any of the foregoing or in NRF2. Evaluation of compounds in such cell lines, or in cell lines harboring mutations affecting mitochondrial metabolism or a mitochondrial folate pathway can also be useful for identifying cell and cancer types in which compounds of the disclosure would be particularly useful, have increased anti-proliferative activity and/or improved activity at a lower dose.

### EXEMPLIFICATION

### Example 1. Pharmacokinetics Study of KDG-0320-0A

A standard pharmacokinetic (PK) study of KDG-0320-0A was performed in male CD1 mice dosed with a single oral, intravenous or intraperitoneal dose of KDG-0320-0A. The parameters of the PK study are described in Table 2. Dosing solutions were freshly made prior to use. No abnormal clinical symptoms were observed during the entire experiment. For IV, PO and IP, after collection, plasma samples from the same time point were pooled, and the pooled samples were analyzed by LC/MS/MS.

TABLE 2

| N/Treatment: | 2 (total 6) | | |
|---|---|---|---|
| Doses: | IV | 1 | mg/kg |
| | PO | 50 | mg/kg |
| | IP | 30 | mg/kg |
| Formulations: | IV | 20% HP-b-CD in water | |
| | PO | 20% HP-b-CD in water | |
| | IP | 20% HP-b-CD in water | |
| Dosing Solutions: | IV | 0.2 | mg/mL |
| | PO | 5 | mg/mL |
| | IP | 6 | mg/mL |

| | | |
|---|---|---|
| Blood Sampling @ | IV | 0.083, 0.25, 0.5, 1, 2, 4, 8 h |
| | PO | 0.25, 0.5, 1, 2, 4, 8 h |
| | IP | 0.25, 0.5, 1, 2, 4, 8 h |

The desired serial concentrations of working solutions were achieved by diluting stock solution of analyte with 50% acetonitrile in water solution. 5 μL of working solutions (2, 4, 10, 20, 100, 200, 1000, 2000 ng/mL) were added to 10 μL of blank CD1 mouse plasma to achieve calibration standards of 1-1000 ng/mL (1, 2, 5, 10, 50, 100, 500, 1000 ng/mL) in a total volume of 15 μL. Four quality control (QC) samples at 2 ng/mL, 5 ng/mL, 50 ng/mL and 800 ng/mL for plasma were prepared independently of those used for the calibration curves. These QC samples were prepared on the day of analysis in the same way as calibration standards.

15 μL standards, 15 μL QC samples and 15 μL unknown samples from the blood sampling (10 μL plasma with 5 μL blank solution) were added to 200 μL of acetonitrile containing IS mixture for precipitating protein. Then, the samples were vortexed for 30 seconds. After centrifugation at 4 degrees Celsius, 4000 rpm for 15 minutes, the supernatant was diluted three times with water. 15 μL of diluted supernatant was injected into the LC/MS/MS system for quantitative analysis. Table 3 describes the parameters of the LC/MS/MS analysis.

TABLE 3

| | |
|---|---|
| HPLC: | Instrument: Shimadzu (DGU-20A5R(C), Serial NO: L20705724128IX; |
| | LC-30AD 220 V Serial NO: L20555713197 AE and L20555713198 AE; |
| | SIL-30AC 220 V, Serial NO: L20565706087 AE), Rack Changer 2220 V |
| | (Serial No. L20585701213SS; CTO-30A 220 V: Serial No. |
| | L20575701515 CD) |
| Column: | Phenomenex Kinetex 5μ C18 100A (50*2.1 mm) |
| MS | AB API 5500 + LC/MS/MS instrument (Serial NO. EX222101912) |
| HPLC Conditions | |
| Mobile Phase | |
| Solution A: | 5% Acetonitrile in Water (0.1% Formic acid) |
| Solution B: | 95% Acetonitrile in Water (0.1% Formic acid) |
| Gradient | |

Flow rate: 0.6 mL/min

| | | |
|---|---|---|
| Time (min) | A (%) | B (%) |
| 0.01 | 90.0 | 10.0 |
| 0.20 | 90.0 | 10.0 |
| 1.50 | 5.00 | 95.0 |
| 1.70 | 5.00 | 95.0 |
| 1.71 | 90.0 | 10.0 |
| 2.00 | 90.0 | 10.0 |

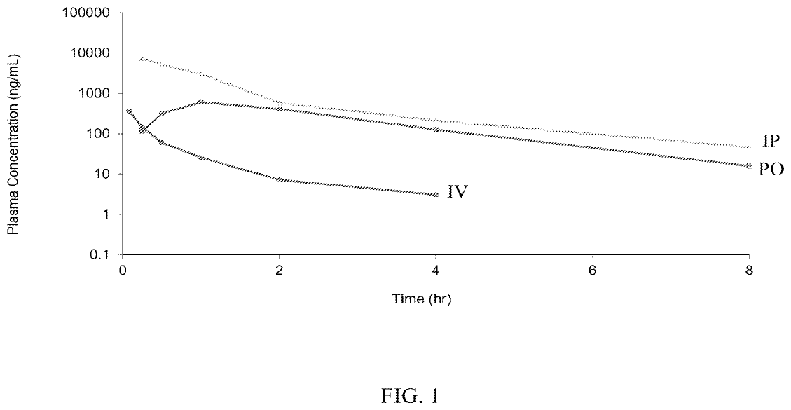

The plasma concentration time data for IV, PO and IP administration of KDG-0320-0A are reported in Tables 4, 5 and 6, respectively. The bolded data were included in terminal elimination T_{1/2} calculations. Summaries of the pharmacokinetic parameters for IV, PO and IP administration of KDG-0320-0A are provided in Tables 7, 8 and 9, respectively. The mean plasma concentration versus time profile for IV, PO and IP administration of KDG-0320-0A is shown in FIG. 1.

TABLE 4

| Time | Concentration (ng/mL) |
|---|---|
| (h) | IV_Mean |
| 0.083 | 363 |
| 0.25 | 144 |
| 0.5 | 60.2 |
| 1 | 25.7 |
| 2 | 7.16 |
| 4 | 3.07 |
| 8 | BLOQ |

TABLE 5

| Time | Concentration (ng/mL) |
|---|---|
| (h) | PO_Mean |
| 0.25 | 117 |
| 0.5 | 315 |
| 1 | 605 |
| 2 | 411 |
| 4 | 126 |
| 8 | 15.8 |

TABLE 6

| Time | Concentration (ng/mL) |
|---|---|
| (h) | IP_Mean |
| 0.25 | 7460 |
| 0.5 | 5240 |
| 1 | 3060 |
| 2 | 589 |
| 4 | 211 |
| 8 | 46.2 |

TABLE 7

| PK parameters | Unit | IV_Mean |
|---|---|---|
| Cl_obs | mL/min/kg | 104 |
| T_{1/2} | h | 1.04 |
| C₀ | ng/ml | 575 |
| AUCₗₐₛₜ | h*ng/mL | 155 |
| AUC_{Inf} | h*ng/mL | 160 |
| AUC_%Extrap_obs | % | 2.88 |
| MRT_{Inf}_obs | h | 0.632 |
| AUCₗₐₛₜ/D | h*mg/mL | 155 |
| Vₛₛ_obs | L/kg | 3.96 |

TABLE 8

| PK parameters | Unit | PO_Mean |
|---|---|---|
| T_{1/2} | h | 1.28 |
| Tₘₐₓ | h | 1.00 |
| Cₘₐₓ | ng/ml | 605 |
| AUCₗₐₛₜ | h*ng/mL | 1627 |
| AUC_{Inf} | h*ng/mL | 1657 |
| AUC_%Extrap_obs | % | 1.77 |
| MRT_{Inf}_obs | h | 2.30 |
| AUCₗₐₛₜ/D | h*mg/mL | 32.5 |
| F | % | 20.8 |

TABLE 9

| PK parameters | Unit | IP_Mean |
|---|---|---|
| T_{1/2} | h | 1.66 |
| Tₘₐₓ | h | 0.250 |
| Cₘₐₓ | ng/ml | 7460 |
| AUCₗₐₛₜ | h*ng/mL | 7734 |
| AUC_{Inf} | h*ng/mL | 7844 |
| AUC_%Extrap_obs | % | 1.41 |
| MRT_{Inf}_obs | h | 1.27 |
| AUCₗₐₛₜ/D | h*mg/mL | 258 |
| F | % | 164 |

BLOQ=Below quantifiable limit of 1 ng/mL. PK parameters were estimated by non-compartmental model using WinNonlin 6.1. The PO bioavailability (F %) was calculated as following:

AUCₗₐₛₜ-PO/AUC_{INF}-PO>80%: F=(AUC_{INF}-PO*DoseIV)/(mean AUC_{INF}-IV*DosePO) AUCₗₐₛₜ-PO/AUC_{INF}-PO≤80% or AUC_{INF} was not available: F=(AUCₗₐₛₜ-PO*DoseIV)/(mean AUCₗₐₛₜ-IV*DosePO).

The IP bioavailability (F %) was calculated as following:

AUCₗₐₛₜ-IP/AUC_{INF}-IP>80%: F=(AUC_{INF}-IP*DoseIV)/(mean AUC_{INF}-IV*DoseIP) AUCₗₐₛₜ-IP/AUC_{INF}-IP≤80% or AUC_{INF} was not available: F=(AUCₗₐₛₜ-IP*DoseIV)/(mean AUCₗₐₛₜ-IV*DoseIP).

### Example 2. Pharmacokinetics Study of KDG-0388

A standard PK study of KDG-0388 was performed in male CD1 mice dosed with a single oral or intraperitoneal dose of KDG-0388. The parameters of the PK study are described in Table 10. Dosing solutions were freshly made prior to use, and were in the form of a suspension. No abnormal clinical symptoms were observed during the entire experiment. For IV and IP, after collection, plasma samples from the same time point were pooled, and the pooled samples were analyzed by LC/MS/MS.

TABLE 10

| N/Treatment: | 2 (total 4) | | |
|---|---|---|---|
| Doses: | PO | 200 | mg/kg |
| | IP | 200 | mg/kg |
| Formulations: | PO | 20% HP-b-CD in water | |
| | IP | 20% HP-b-CD in water | |
| Dosing Solutions: | PO | 20 | mg/mL |
| | IP | 40 | mg/mL |

| | | |
|---|---|---|
| Blood Sampling @ | PO | 0.25, 0.5, 1, 2, 4, 8, 16, 24 h |
| | IP | 0.25, 0.5, 1, 2, 4, 8, 16, 24 h |

The desired serial concentrations of working solutions were achieved by diluting stock solution of analyte with 50% acetonitrile in water solution. 5 μL of working solutions (2, 4, 10, 20, 100, 400, 2000, 4000 ng/mL) were added to 10 μL of blank CD1 mouse plasma to achieve calibration standards of 1-2000 ng/mL (1, 2, 5, 10, 50, 200, 1000, 2000 ng/mL) in a total volume of 15 μL. Five quality control (QC) samples at 2 ng/mL, 5 ng/mL, 50 ng/mL, 800 ng/mL and 1600 ng/mL for plasma were prepared independently of those used for the calibration curves. These QC samples were prepared on the day of analysis in the same way as calibration standards.

15 μL standards, 15 μL QC samples and 15 μL unknown samples (10 μL plasma with 5 μL blank solution) were added to 200 μL of acetonitrile containing IS mixture for precipitating protein. Then, the samples were vortexed for 30 seconds. After centrifugation at 4 degrees Celsius, 4000 rpm for 15 minutes, the supernatant was diluted three times with water. 25 μL of diluted supernatant was injected into the LC/MS/MS system for quantitative analysis. Table 11 describes the parameters of the LC/MS/MS analysis.

TABLE 11

| | |
|---|---|
| HPLC: | Instrument: Shimadzu (DGU-20A5R(C), Serial NO: L20705724128IX; |
| | LC-30AD 220 V Serial NO: L20555713197 AE and L20555713198 AE; |
| | SIL-30AC 220 V, Serial NO: L20565706087 AE), Rack Changer 2220 V |
| | (Serial No. L20585701213SS; CTO-30A 220 V: Serial |
| | No.L20575701515 CD) |
| Column: | Phenomenex Kinetex 5μ C18 100A (50*2.1 mm) |
| MS | AB API 5500 + LC/MS/MS instrument (Serial NO. EX222101912) |
| HPLC Conditions | |
| Mobile Phase | |
| Solution A: | 5% Acetonitrile in Water (0.1% Formic acid) |
| Solution B: | 95% Acetonitrile in Water (0.1% Formic acid) |
| Gradient | |

Flow rate: 0.6 mL/min

| | | |
|---|---|---|
| Time (min) | A (%) | B (%) |
| 0.01 | 95.0 | 5.00 |
| 0.20 | 95.0 | 5.00 |
| 1.90 | 5.00 | 95.0 |
| 2.20 | 5.00 | 95.0 |
| 2.21 | 95.0 | 5.00 |
| 2.50 | 95.0 | 5.00 |

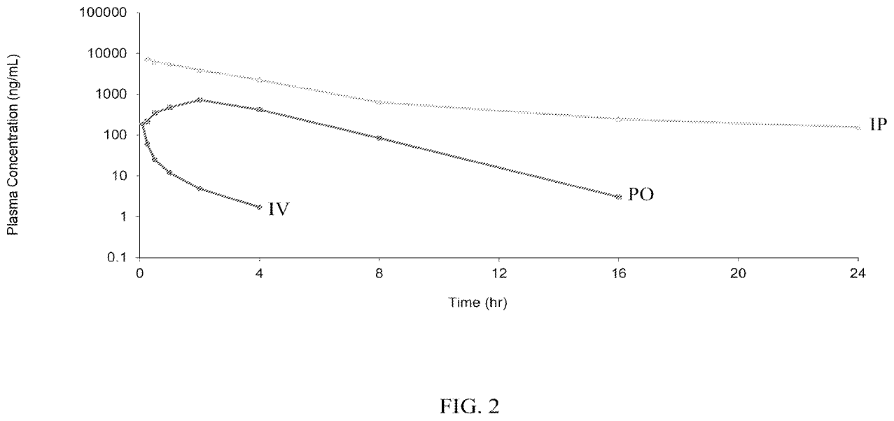

The plasma concentration time data for IV (1 mg/kg), PO and IP administration of KDG-0388 are reported in Tables 12, 13 and 14, respectively. The bolded data were included in terminal elimination T_{1/2} calculations. Summaries of the pharmacokinetic parameters for IV, PO and IP administration of KDG-0388 are provided in Tables 15, 16 and 17, respectively. The mean plasma concentration versus time profile for IV, PO and IP administration of KDG-0388 is shown in FIG. 2.

TABLE 12

| Time | Concentration (ng/mL) |
|---|---|
| (h) | IV_Mean |
| 0.083 | 186 |
| 0.25 | 60.6 |
| 0.5 | 24.9 |
| 1 | 11.8 |
| 2 | 4.86 |
| 4 | 1.71 |
| 8 | BLOQ |

TABLE 13

| Time | Concentration (ng/mL) |
|---|---|
| (h) | PO_Mean |
| 0.25 | 215 |
| 0.5 | 347 |
| 1 | 475 |
| 2 | 719 |
| 4 | 417 |
| 8 | 84.3 |
| 16 | 3.04 |
| 24 | BLOQ |

TABLE 14

| Time | Concentration (ng/mL) |
|---|---|
| (h) | IP_Mean |
| 0.25 | 7520 |
| 0.5 | 6170 |
| 1 | 5460 |
| 2 | 3910 |
| 4 | 2230 |
| 8 | 633 |
| 16 | 247 |
| 24 | 155 |

TABLE 15

| PK parameters | Unit | IV_Mean |
|---|---|---|
| Cl_obs | mL/min/kg | 210 |
| T_{1/2} | h | 1.11 |
| C₀ | ng/ml | 325 |
| AUCₗₐₛₜ | h*ng/mL | 76.6 |
| AUC_{Inf} | h*ng/mL | 79.3 |
| AUC_%Extrap_obs | % | 3.44 |
| MRT_{Inf}_obs | h | 0.698 |
| AUCₗₐₛₜ/D | h*mg/mL | 76.6 |
| Vₛₛ_obs | L/kg | 8.80 |

TABLE 16

| PK parameters | Unit | PO_Mean |
|---|---|---|
| T_{1/2} | h | 1.69 |
| Tₘₐₓ | h | 2.00 |
| Cₘₐₓ | ng/ml | 719 |
| AUCₗₐₛₜ | h*ng/mL | 3388 |
| AUC_{Inf} | h*ng/mL | 3395 |
| AUC_%Extrap_obs | % | 0.218 |
| MRT_{Inf}_obs | h | 3.53 |
| AUCₗₐₛₜ/D | h*mg/mL | 16.9 |
| F | % | 21.4 |

TABLE 17

| PK parameters | Unit | IP_Mean |
|---|---|---|
| T_{1/2} | h | 7.88 |
| Tₘₐₓ | h | 0.250 |
| Cₘₐₓ | ng/ml | 7520 |
| AUCₗₐₛₜ | h*ng/mL | 27238 |
| AUC_{Inf} | h*ng/mL | 29000 |
| AUC_%Extrap_obs | % | 6.08 |
| MRT_{Inf}_obs | h | 6.33 |
| AUCₗₐₛₜ/D | h*mg/ml | 136 |
| F | % | 183 |

BLOQ=Below quantifiable limit of 1 ng/mL. PK parameters were estimated by non-compartmental model using WinNonlin 6.1. The PO bioavailability (F %) was calculated as following:

AUCₗₐₛₜ-PO/AUC_{INF}-PO>80%: F=(AUC_{INF}-PO*DoseIV)/(mean AUC_{INF}-IV*DosePO) AUCₗₐₛₜ-PO/AUC_{INF}-PO≤80% or AUC_{INF} was not available: F=(AUCₗₐₛₜ-PO*DoseIV)/(mean AUCₗₐₛₜ-IV*DosePO).

The IP bioavailability (F %) was calculated as following:

AUCₗₐₛₜ-IP/AUC_{INF}-IP>80%: F=(AUC_{INF}-IP*DoseIV)/(mean AUC_{INF}-IV*DoseIP) AUCₗₐₛₜ-IP/AUC_{INF}-IP≤80% or AUC_{INF} was not available: F=(AUCₗₐₛₜ-IP*DoseIV)/(mean AUClast-IV*DoseIP).

### Example 3. Pharmacokinetics Study of KDG-0271

A standard PK study of KDG-0271 was performed in male CD1 mice dosed with a single oral or intraperitoneal dose of KDG-0271. The parameters of the PK study are described in Table 18. Dosing solutions were freshly made prior to use, and were in the form of a suspension. No abnormal clinical symptoms were observed during the entire experiment. For IV and IP, after collection, plasma samples from the same time point were pooled, and the pooled samples were analyzed by LC/MS/MS.

TABLE 18

| N/Treatment: | 2 (total 4) | | |
|---|---|---|---|
| Doses: | | | |
| | PO | 200 | mg/kg |
| | IP | 200 | mg/kg |
| Formulations: | | | |
| | PO | 5% DMSO, 95% “8% HP-b-CD in water” | |
| | IP | 5% DMSO, 95% “8% HP-b-CD in water“ | |
| Dosing Solutions: | | | |
| | PO | 40 | mg/mL |
| | IP | 40 | mg/mL |
| Blood Sampling @ | | | |

| | |
|---|---|
| PO | 0.25, 0.5, 1, 2, 4, 8 h |
| IP | 0.25, 0.5, 1, 2, 4, 8 h |

The desired serial concentrations of working solutions were achieved by diluting stock solution of analyte with 50% acetonitrile in water solution. 5 μL of working solutions (1, 2, 4, 20, 100, 200, 1000, 2000 ng/mL) were added to 10 μL of blank CD1 mouse plasma to achieve calibration standards of 0.511000 ng/mL (0.5, 1, 2, 10, 50, 100, 500, 1000 ng/mL) in a total volume of 15 μL. Four quality control samples at 1 ng/mL, 2 ng/mL, 50 ng/mL and 800 ng/mL for plasma were prepared independently of those used for the calibration curves. These QC samples were prepared on the day of analysis in the same way as calibration standards.

15 μL standards, 15 μL QC samples and 15 μL unknown samples (10 μL plasma with 5 μL blank solution) were added to 200 μL of acetonitrile containing IS mixture for precipitating protein. Then the samples were vortexed for 30 seconds. After centrifugation at 4 degrees Celsius, 4000 rpm for 15 minutes, the supernatant was diluted three times with water. 10 μL of diluted supernatant was injected into the LC/MS/MS system for quantitative analysis. Table 19 describes the parameters of the LC/MS/MS analysis.

TABLE 19

| | |
|---|---|
| HPLC: | Instrument: Shimadzu (DGU-20A5R(C), Serial NO: L20705622078 IX; |
| | LC-30AD Serial NO: L20555612287 AE and L20555612284 AE; SIL- |
| | 30AC, Serial NO: L20565605653 AE), Rack Changer II (Serial No. |
| | L20585601155 SS; CBM-20A: Serial No.L20235636584 CD); CTO- |
| | 30A: Serial No.L20575601418 CD) |
| Column: | Phenomenex Kinetex 5μ C18 100A (50*2.1 mm) |
| MS | AB API 5500 LC/MS/MS instrument (Serial NO. EF221671902) |
| HPLC Conditions | |
| Mobile Phase | |
| Solution A: | 5% Acetonitrile in Water (0.1% Formic acid) |
| Solution B: | 95% Acetonitrile in Water (0.1% Formic acid) |
| Gradient | |

Flow rate: 0.6 mL/min

| | | |
|---|---|---|
| Time (min) | A (%) | B (%) |
| 0.01 | 100 | 0.00 |
| 0.20 | 100 | 0.00 |
| 1.50 | 0.00 | 100 |
| 1.70 | 0.00 | 100 |
| 1.71 | 100 | 0.00 |
| 2.00 | 100 | 0.00 |

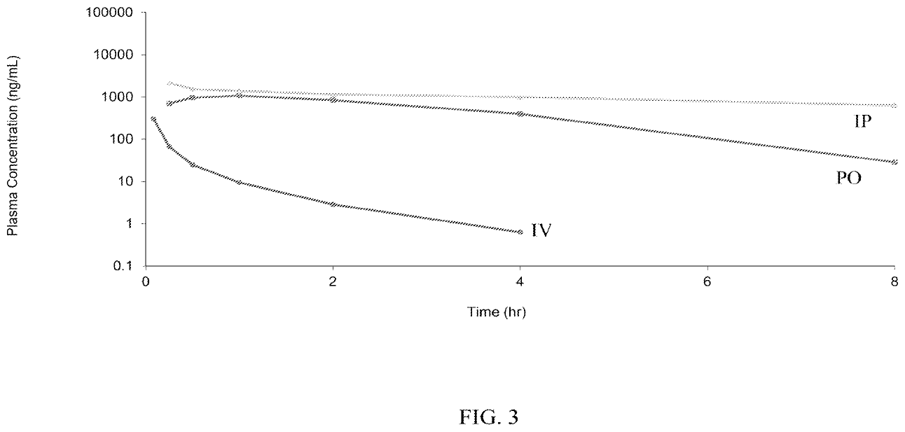

The plasma concentration time data for IV (1 mg/kg), P and IP administration of KDG-0271 are reported in Tables 20, 21 and 22, respectively. The bolded data were included in terminal elimination T_{1/2} calculations. Summaries of the pharmacokinetic parameters for IV, PO and IP administration of KDG-0271 are provided in Tables 23, 24 and 25, respectively. The mean plasma concentration versus time profile for IV, PO and IP administration of KDG-0271 is shown in FIG. 3.

TABLE 20

| Time | Concentration (ng/mL) |
|---|---|
| (h) | IV_Mean |
| 0.083 | 301 |
| 0.25 | 66.6 |
| 0.5 | 24.8 |
| 1 | 9.44 |
| 2 | 2.87 |
| 4 | 0.632 |
| 8 | BLOQ |

TABLE 21

| Time | Concentration (ng/mL) |
|---|---|
| (h) | PO_Mean |
| 0.25 | 701 |
| 0.5 | 958 |
| 1 | 1070 |
| 2 | 836 |
| 4 | 395 |
| 8 | 28.6 |

TABLE 22

| Time | Concentration (ng/mL) |
|---|---|
| (h) | IP_Mean |
| 0.25 | 2160 |
| 0.5 | 1530 |
| 1 | 1390 |
| 2 | 1160 |
| 4 | 992 |
| 8 | 638 |

TABLE 23

| PK parameters | Unit | IV_Mean |
|---|---|---|
| Cl_obs | mL/min/kg | 167 |
| T_{1/2} | h | 0.787 |
| C₀ | ng/ml | 637 |
| AUCₗₐₛₜ | h*ng/mL | 99.3 |
| AUC_{Inf} | h*ng/mL | 100 |
| AUC_%Extrap_obs | % | 0.718 |
| MRT_{Inf}_obs | h | 0.332 |
| AUCₗₐₛₜ/D | h*mg/mL | 99.3 |
| Vₛₛ_obs | L/kg | 3.32 |

TABLE 24

| PK parameters | Unit | PO_Mean |
|---|---|---|
| T_{1/2} | h | 1.20 |
| Tₘₐₓ | h | 1.00 |
| Cₘₐₓ | ng/ml | 1070 |
| AUCₗₐₛₜ | h*ng/mL | 3833 |
| AUC_{Inf} | h*ng/mL | 3883 |
| AUC_%Extrap_obs | % | 1.28 |
| MRT_{Inf}_obs | h | 2.37 |
| AUCₗₐₛₜ/D | h*mg/mL | 19.2 |
| F | % | 19.4 |

TABLE 25

| PK parameters | Unit | IP_Mean |
|---|---|---|
| T_{1/2} | h | 6.85 |
| Tₘₐₓ | h | 0.250 |
| Cₘₐₓ | ng/ml | 2160 |
| AUCₗₐₛₜ | h*ng/mL | 8148 |
| AUC_{Inf} | h*ng/mL | 14455 |
| AUC_%Extrap_obs | % | 43.6 |
| MRT_{Inf}_obs | h | 9.67 |
| AUCₗₐₛₜ/D | h*mg/mL | 40.7 |
| F | % | 41.0 |

BLOQ=Below quantifiable limit of 1 ng/mL. PK parameters were estimated by non-compartmental model using WinNonlin 6.1. The PO bioavailability (F %) was calculated as following:

AUCₗₐₛₜ-PO/AUC_{INF}-PO>80%: F=(AUC_{INF}-PO*DoseIV)/(mean AUC_{INF}-IV*DosePO) AUCₗₐₛₜ-PO/AUC_{INF}-PO≤80% or AUC_{INF} was not available: F=(AUCₗₐₛₜ-PO*DoseIV)/(mean AUCₗₐₛₜ-IV*DosePO).

The IP bioavailability (F %) was calculated as following:

AUCₗₐₛₜ-IP/AUC_{INF}-IP>80%: F=(AUC_{INF}-IP*DoseIV)/(mean AUC_{INF}-IV*DoseIP) AUCₗₐₛₜ-IP/AUC_{INF}-IP≤80% or AUC_{INF} was not available: F=(AUCₗₐₛₜ-IP*DoseIV)/(mean AUCₗₐₛₜ-IV*DoseIP).

### Example 4. Pharmacokinetics Study of KDG-0271

A standard PK study of KDG-0271 was performed in male CD1 mice dosed with a single intravenous, oral or intraperitoneal dose of KDG-0271. The parameters of the PK study are described in Table 26. Dosing solutions were freshly made prior to use, and were in the form of a suspension. No abnormal clinical symptoms were observed during the entire experiment. For IV and IP, after collection, plasma samples from the same time point were pooled, and the pooled samples were analyzed by LC/MS/MS.

TABLE 26

| N/Treatment: | 2 (total 6) | | |
|---|---|---|---|
| Doses: | IV | 1 | mg/kg |
| | PO | 50 | mg/kg |
| | IP | 30 | mg/kg |
| Formulations: | IV | 5% DMSO, 95% | |
| | | “8%HP-b-CD in water” | |
| | PO | 5% DMSO, 95% | |
| | | “8%HP-b-CD in water” | |
| | IP | 5% DMSO, 95% | |
| | | “8%HP-b-CD in water” | |
| Dosing | IV | 0.2 | mg/mL |
| Solutions: | PO | 5 | mg/mL |
| | IP | 6 | mg/mL |
| Blood | IV | 0.083, 0.25, 0.5, 1, 2, 4, 8 | h |
| Sampling @ | PO | 0.25, 0.5, 1, 2, 4, 8 | h |
| | IP | 0.25, 0.5, 1, 2, 4, 8 | h |

Sample preparation and analysis were conducted in accordance with the method described in Example 3.

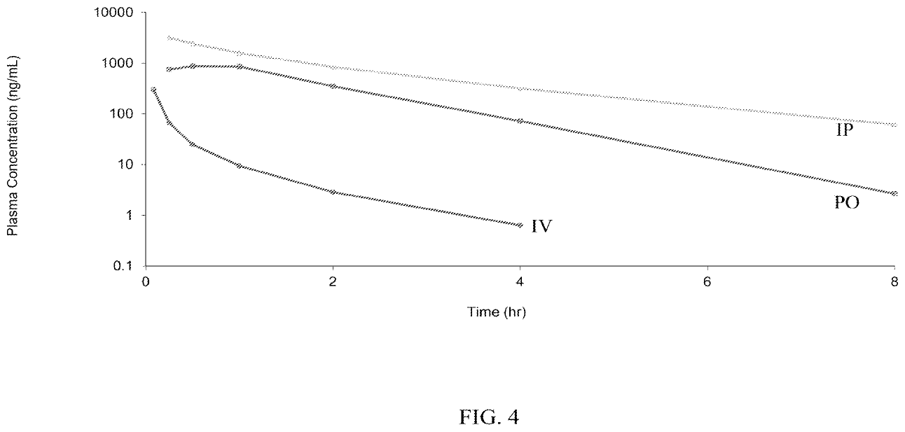

The plasma concentration time data for PO and IP administration of KDG-0271 are reported in Tables 27 and 28, respectively. The bolded data were included in terminal elimination T_{1/2} calculations. Summaries of the pharmacokinetic parameters for PO and IP administration of KDG-0271 are provided in Tables 29 and 30, respectively. The plasma concentration time data and pharmacokinetic parameters for IV administration of KDG-0271 are reported in Example 3. The mean plasma concentration versus time profile for PO and IP administration of KDG-0271 is shown in FIG. 4.

TABLE 27

| Time | Concentration (ng/ml) |
|---|---|
| (h) | PO_Mean |
| 0.25 | 750 |
| 0.5 | 872 |
| 1 | 857 |
| 2 | 350 |
| 4 | 72.0 |
| 8 | 2.67 |

TABLE 28

| Time | Concentration (ng/mL) |
|---|---|
| (h) | IP_Mean |
| 0.25 | 3170 |
| 0.5 | 2420 |
| 1 | 1580 |
| 2 | 841 |
| 4 | 317 |
| 8 | 61.4 |

TABLE 29

| PK parameters | Unit | PO_Mean |
|---|---|---|
| T_{1/2} | h | 0.851 |
| Tₘₐₓ | h | 0.500 |
| Cₘₐₓ | ng/ml | 872 |
| AUCₗₐₛₜ | h*ng/mL | 1904 |
| AUC_{Inf} | h*ng/mL | 1907 |
| AUC_%Extrap_obs | % | 0.172 |
| MRT_{Inf}_obs | h | 1.49 |
| AUCₗₐₛₜ/D | h*mg/mL | 38.1 |
| F | % | 38.1 |

TABLE 30

| PK parameters | Unit | IP_Mean |
|---|---|---|
| T_{1/2} | h | 1.60 |
| Tₘₐₓ | h | 0.250 |
| Cₘₐₓ | ng/ml | 3170 |
| AUCₗₐₛₜ | h*ng/mL | 5220 |
| AUC_{Inf} | h*ng/mL | 5362 |
| AUC_%Extrap_obs | % | 2.65 |
| MRT_{Inf}_obs | h | 1.98 |
| AUCₗₐₛₜ/D | h*mg/mL | 174 |
| F | % | 179 |

BLOQ=Below quantifiable limit of 1 ng/mL. PK parameters were estimated by non-compartmental model using WinNonlin 6.1. The PO bioavailability (F %) was calculated as following:

AUCₗₐₛₜ-PO/AUC_{INF}-PO>80%: F=(AUC_{INF}-PO*DoseIV)/(mean AUC_{INF}-IV*DosePO) AUCₗₐₛₜ-PO/AUC_{INF}-PO≤80% or AUC_{INF} was not available: F=(AUCₗₐₛₜ-PO*DoseIV)/(mean AUCₗₐₛₜ-IV*DosePO).

The IP bioavailability (F %) was calculated as following:

AUCₗₐₛₜ-IP/AUC_{INF}-IP>80%: F=(AUC_{INF}-IP*DoseIV)/(mean AUC_{INF}-IV*DoseIP) AUCₗₐₛₜ-IP/AUC_{INF}-IP≤80% or AUC_{INF} was not available: F=(AUCₗₐₛₜ-IP*DoseIV)/(mean AUCₗₐₛₜ-IV*DoseIP).

### Example 5. HCT116 Growth Inhibition and Formate Rescue Assays

3,000 Cells were plated in each well of a 96-well plate with 80 μL of DMEM media supplemented with 10% dFBS, and incubated for 24 hours. Drugs with or without sodium formate (1 mM) were then added to each well to a final volume of 100 μL. Cell growth was measured as fluorescence intensity by resazurin after four days. In short, resazurin was added to each well to a final concentration of 10 μg·mL⁻¹. After incubation for one hour, fluorescence was measured by a Synergy HT plate reader (BioTek Instruments).

### Example 6. Mouse Liver Microsome Assay

Metabolic stability of several compounds in mouse liver microsomes was tested. Briefly, a master solution was prepared according to Table 31.

TABLE 31

| | Stock | | | Final | |
|---|---|---|---|---|---|
| Reagent | Concentration | | Volume | Concentration | |
| Phosphate buffer | 200 | mM | 200 μL | 100 | mM |
| Ultra-pure H₂O | — | | 64 μL | — | |
| MgCl₂ solution | 50 | mM | 40 μL | 5 | mM |
| Alamethacin | 5 | mg/mL | 2 μL | 25 | ug/mL |
| Microsomes | 20 | mg/mL | 10 μL | 0.5 | mg/mL |

Two separate experiments were performed: an experiment with cofactors and an experiment without cofactors. In the experiment with cofactors 40 μL of 10 mM NADPH and 40 μL of 20 mM UDPGA were added to the incubations. The final concentrations of microsomes, NADPH and UDPGA were 0.5 mg/mL, 1 mM and 2 mM, respectively. In the experiment without cofactors 10 μL of 20 mg/mL liver microsomes and 80 μL of ultra-pure H₂O were added to the incubations. The final concentration of microsomes was 0.5 mg/mL. The mixture was pre-warmed at 37° C. for 5 minutes.

The reaction was started with the addition of 4 μL of 200 μM control compound or test compound solutions. Verapamil was used as positive control in this study. The final concentration of test compound was 2 μM and the final concentration of control compound was 1 μM. The incubation solution was incubated in water bath at 37° C.

Aliquots of 50 μL were taken from the reaction solution at 0.5, 5, 15, 30, 45 and 60 minutes. The reaction was stopped by the addition of five volumes of cold acetonitrile with IS (100 nM alprazolam, 200 nM caffeine and 100 nM tolbutamide). Samples were centrifuged at 3,220 g for 40 minutes. Aliquot of 100 μL of the supernatant was mixed with 100 μL of ultra-pure H₂O and then subject to LC-MS/MS analysis.

All calculations were carried out using Microsoft Excel. Peak areas were determined from extracted ion chromatograms. The slope value, k, was determined by linear regression of the natural logarithm of the remaining percentage of the parent drug versus incubation time curve. The in vitro half-life (in vitro t½) was determined from the slope value:

in vitro t_{1/2}=−(0.693/k).

Conversion of the in vitro t_{1/2}(min) into the in vitro intrinsic clearance (in vitro CLᵢₙₜ, in μL/min/mg protein) was done using the following equation (mean of duplicate determinations):

$$in vitro CL int = ( \frac{0.693}{( t 1 / 2 )} ) ( \frac{volume of incubation ( μL )}{amount of proteins ( mg )} ) .$$

The rules for data processing are shown in Table 32.

TABLE 32

| Remaining | |
|---|---|
| % | Processing Rules |
| ≥80% | When the calculated Clᵢₙₜ value > 0, report the |
| at 60 | calculated CLᵢₙₜ value. When the calculated |
| min | Clᵢₙₜ value < 0, then report “0” instead of |
| | calculated value. All the other data points |
| | should fall in the range of 80%-120% (one data |
| | point within the range of 70%-130% is accepted, |
| | otherwise the experiment should be repeated). |
| <80% | Always remove from the calculation all points with < |
| at 60 | 10% left of 0.5 min sample, but leave at least 2 points |
| min | If T-test with p < 0.05 is obtained, report the calculated |
| | CLᵢₙₜ value. If T-test with p < 0.05 is not obtained, |
| | the experiment must be repeated. |

### Example 7. Representative Syntheses

KDG-0271-0 was synthesized using the synthetic route depicted in Scheme 1.

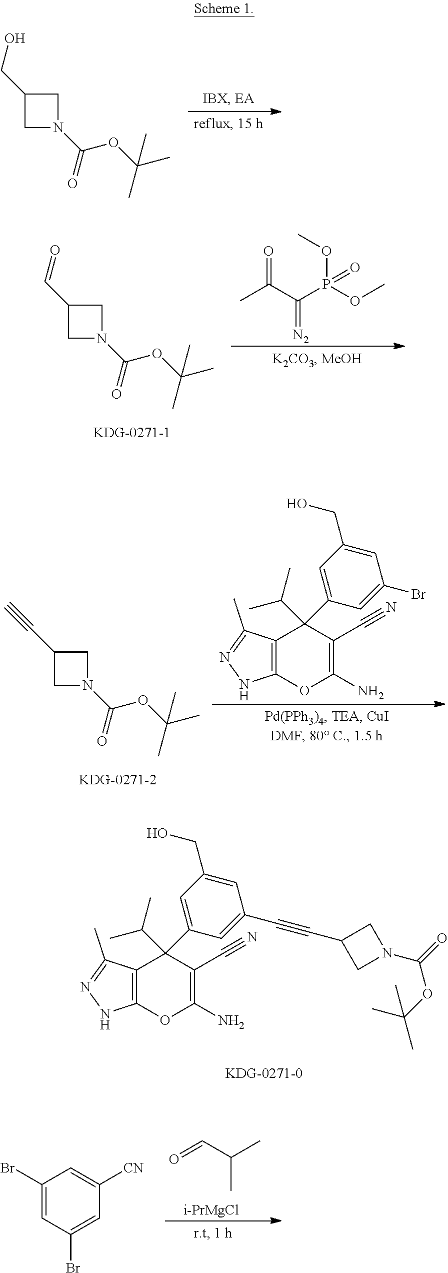

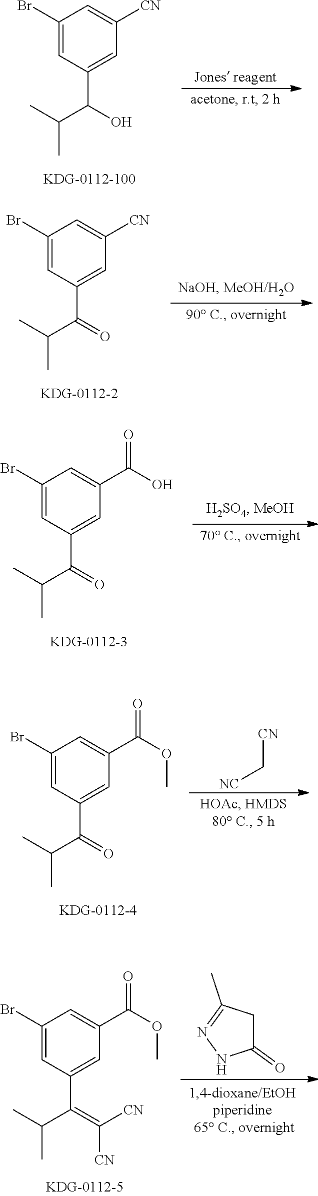

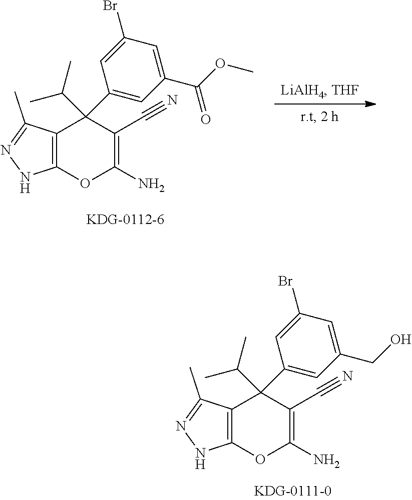 [SMILES: COC(=O)c1cc(Br)cc(C2(C(C)C)C(C#N)=C(N)Oc3[nH]nc(C)c32)c1 ; Cc1n[nH]c2c1C(c1cc(Br)cc(CO)c1)(C(C)C)C(C#N)=C(N)O2]

Synthesis of 3-bromo-5-(1-hydroxy-2-methylpropyl)benzonitrile. Into a 5000-mL, 3-necked, round-bottomed flask purged and maintained with an inert atmosphere of nitrogen was placed 3,5-dibromobenzonitrile (300 g, 1149.795 mmol, 1 equiv) and THF (2500 mL). This was followed by the addition of i-PrMgCl (579 mL, 5629.777 mmol, 1.00 equiv, 2M) dropwise with stirring at 0° C. The resulting solution was stirred for 1 hour (hr) at room temperature. To this was added a solution of 2-methylpropanal (91.20 g, 1264.775 mmol, 1.10 equiv) in THE (700 mL) dropwise with stirring at room temperature. The resulting solution was stirred for 1 hr at room temperature. The reaction was then quenched by the addition of 500 mL of NH₄Cl (aq). The resulting solution was extracted with 3×2000 mL of ethyl acetate and the organic layers combined and dried over anhydrous sodium sulfate and concentrated. The residue was applied onto a silica gel column with ethyl acetate/petroleum ether (1:10). This resulted in 250 g (85%) of 3-bromo-5-(1-hydroxy-2-methylpropyl)benzonitrile as yellow oil. ¹H NMR (300 MHz, Chloroform-d) δ 7.72 (d, J=11.0 Hz, 1H), 7.62-7.51 (m, 1H), 4.48 (d, J=5.9 Hz, 1H), 0.92 (dd, J=14.8, 6.8 Hz, 6H).

Synthesis of 3-bromo-5-(2-methylpropanoyl)benzonitrile. Into a 3000-mL, 3-necked, round-bottomed flask purged and maintained with an inert atmosphere of nitrogen was placed 3-bromo-5-(1-hydroxy-2-methylpropyl)benzonitrile (255 g, 1003.435 mmol, 1 equiv) and acetone (2500 mL). This was followed by the addition of Jones reagent (294 mL) dropwise with stirring at room temperature. The resulting solution was stirred for 2 hr at room temperature. The reaction was then quenched by the addition of 1000 mL of water. The resulting solution was extracted with 3×1000 mL of ethyl acetate and the organic layers combined and dried over anhydrous sodium sulfate and concentrated. The residue was applied onto a silica gel column and eluted with ethyl acetate/petroleum ether (1.10). This resulted in 241 g (80%) of 3-bromo-5-(2-methylpropanoyl)benzonitrile as a white solid. ¹H NMR (300 MHz, Chloroform-d) δ 8.29 (t, J=1.7 Hz, 1H), 8.15 (t, J=1.5 Hz, 1H), 7.97 (t, J=1.7 Hz, 1H), 3.52-3.43 (m, 1H), 1.26 (d, J=6.9 Hz, 6H).

Synthesis of 3-bromo-5-(2-methylpropanoyl) benzoic acid. Into a 3000-mL, round-bottomed flask was placed 1-bromo-3-isocyano-5-(2-methylpropanoyl) benzene (280 g, 1.00 equiv) in MeOH (1000.00 mL) and NaOH (1000 mL, 4 moL/L). The resulting solution was stirred overnight at 90° C. The resulting mixture was concentrated. The pH value of the solution was adjusted to 6 with HCl (12 mol/L). The solids were collected by filtration. The solid was dried under infrared light. This resulted in 250 g (crude) of 3-bromo-5-(2-methylpropanoyl) benzoic acid as a white solid. ¹H NMR (300 MHz, DMSO-d6) δ 8.39 (t, J=1.5 Hz, 1H), 8.23 (t, J=1.8 Hz, 1H), 8.16 (t, J=1.8 Hz, 1H), 3.72-3.63 (m, 1H), 1.11 (d, J=6.6 Hz, 6H).

Synthesis of methyl 3-bromo-5-(2-methylpropanoyl)benzoate. Into a 5-L, 3-necked, round-bottomed flask was placed 3-bromo-5-(2-methylpropanoyl)benzoic acid (230 g, 846 mmol, 1.00 equiv) in MeOH (2300.00 mL). Concentrated H₂SO₄ (250.00 mL) was added dropwise with stirring at room temperature. The resulting solution was stirred overnight at 80° C. The resulting mixture was concentrated. The reaction was poured into 200 g of ice. The solids were collected by filtration. The solid was dried under infrared light. This resulted in 250 g pure of methyl 3-bromo-5-(2-methylpropanoyl)benzoate as a white solid. ¹H NMR (300 MHz, DMSO-d6) δ8.40-8.36 (m, 2H), 8.28 (t, J=1.7 Hz, 1H), 3.91 (s, 3H), 3.82-3.63 (m, 1H), 1.11 (d, J=6.6 Hz, 6H).

Synthesis of methyl 3-bromo-5-(1,1-diisocyano-3-methylbut-1-en-2-yl)benzoate. Into a 2000-mL, 3-necked, round-bottomed flask was placed methyl 3-bromo-5-(2-methylpropanoyl) benzoate (115.00 g, 403.315 mmol, 1.00 equiv) in HOAc (500.00 mL), diisocyanomethane (133.22 g, 2016.574 mmol, 5.00 equiv) and HMDS (196.58 g, 1218.011 mmol, 3.02 equiv). The resulting solution was stirred for 5 hr at 80° C. This reaction was repeated in parallel for another one, and the two reactions were worked up together. The reaction mixture was poured in to 300 g of water/ice. The solids were collected by filtration. The solid was dried under infrared light. This resulted in 250 g (80%) of methyl 3-bromo-5-(1,1-diisocyano-3-methylbut-1-en-2-yl)benzoate as an orange solid. ¹H NMR (300 MHz, DMSO-d6) δ 8.21 (t, J=1.5 Hz, 1H), 7.94 (dt, J=7.1, 1.6 Hz, 2H), 3.91 (s, 3H), 3.35-3.26 (m, 2H), 1.10 (d, J=6.6 Hz, 6H).

Synthesis of methyl 3-[6-amino-5-cyano-4-isopropyl-3-methyl-1H-pyrano[2,3-c]pyrazol-4-yl]-5-bromobenzoate. Into a 1000 mL, 3-necked, round-bottomed flask was placed methyl 3-bromo-5-(1,1-dicyano-3-methylbut-1-en-2-yl)benzoate (50.00 g, 150.067 mmol, 1.00 equiv) in dioxane (200.00 mL) and EtOH (200.00 mL), piperidine (14.00 g, 0.164 mmol) and 3-methyl-5-pyrazolone (15.00 g, 0.153 mmol). The resulting solution was stirred overnight at 65° C. The reaction was repeated 3 times. The resulting mixture was concentrated. The residue was applied onto a silica gel column with ethyl acetate/petroleum ether (1:2). The crude product was purified by recrystallization with ethyl acetate (EA). This resulted in 120 g (40%) of methyl 3-[6-amino-5-cyano-4-isopropyl-3-methyl-1H-pyrano[2,3-c]pyrazol-4-yl]-5-bromobenzoate as a white solid. ¹H NMR (300 MHz, DMSO-d6) δ 12.24 (s, 1H), 7.96 (t, J=1.5 Hz, 1H), 7.87 (s, 1H), 7.74 (t, J=1.8 Hz, 1H), 7.00 (s, 2H), 3.86 (s, 3H), 2.79-2.70 (m, 1H), 1.78 (s, 3H), 0.90 (d, J=6.6 Hz, 3H), 0.78 (d, J=6.6 Hz, 3H).

Synthesis of 6-amino-4-[3-bromo-5-(hydroxymethyl)phenyl]-4-isopropyl-3-methyl-1H-pyrano[2,3-c]pyrazole-5-carbonitrile. Into a 1000-mL, 3-necked, round-bottomed flask was placed methyl 3-[6-amino-5-cyano-4-isopropyl-3-methyl-1H-pyrano[2,3-c] pyrazol-4-yl]-5-bromobenzoate (50.00 g, 115,931 mmol, 1.00 equiv) and THF (500.00 mL). This was followed by the addition of LiAlH₄ (8.80 g, 231.863 mmol, 2.00 equiv) in portions at room temperature. The resulting solution was stirred for 2 hr at room temperature. Another one reaction was repeated in parallel, and the two reactions were worked up together. The reaction was then quenched by the addition of 10 mL of water. The pH value of the solution was adjusted to 6 with HCl (1 mol/L). The resulting solution was extracted with 3×1000 mL of ethyl acetate and the organic layers combined and dried over anhydrous sodium sulfate. The residue was applied onto a silica gel column with ethyl acetate/petroleum ether (1.0). This resulted in 59 g (78%) of 6-amino-4-[3-bromo-5-(hydroxymethyl)phenyl]-4-isopropyl-3-methyl-1H-pyrano[2,3-c]pyrazole-5-carbonitrile as a white solid. ¹H NMR (300 MHz, DMSO-d6) δ 12.18 (s, 1H), 7.36 (s, 1H), 7.29 (d, J=1.8 Hz, 2H), 6.88 (s, 2H), 5.33 (t, J=5.7 Hz, 1H), 4.48 (d, J=5.7 Hz, 2H), 2.75-2.66 (m, 1H), 1.73 (s, 3H), 0.89 (d, J=6.3 Hz, 3H), 0.76 (d, J=6.3 Hz, 3H).

Synthesis of tert-butyl 3-formylazetidine-1-carboxylate. Into a 3-L, 4-necked, round-bottomed flask was placed tert-butyl 3-(hydroxymethyl)azetidine-1-carboxylate (100.00 g, 534.077 mmol, 1.00 equiv), EA (1000 mL) and 2-iodoxybenzoic acid (IBX) (299.10 g, 1068.154 mmol, 2.00 equiv). The resulting solution was heated to reflux for 15 hr. The reaction mixture was cooled to room temperature. Petroleum ether (PE) was added to the mixture. The solids were filtered out. The filtrate was concentrated. This resulted in 114 g (crude) of tert-butyl 3-formylazetidine-1-carboxylate as yellow oil.

Synthesis of tert-butyl 3-ethynylazetidine-1-carboxylate. Into a 2000-mL, round-bottomed flask was placed tert-butyl 3-formylazetidine-1-carboxylate (55.00 g, 296.939 mmol, 1.00 equiv), MeOH (500.00 mL), K₂CO₃ (82.08 g, 593.879 mmol, 2.00 equiv) and Seyferth-Gilbert homologation reagent (62.75 g, 326.633 mmol, 1.10 equiv). The resulting solution was stirred for 12 hr at room temperature. The reaction was then quenched by the addition of water. The resulting solution was extracted with ethyl acetate, and dried over anhydrous sodium sulfate. The solids were filtered out. The resulting mixture was concentrated. The residue was applied onto a silica gel column with ethyl acetate/petroleum ether (1:5). This resulted in 45 g (83.62%) of tert-butyl 3-ethynylazetidine-1-carboxylate as a colorless oil.

Synthesis of tert-butyl 3-[2-(3-[6-amino-5-cyano-4-isopropyl-3-methyl-11H-pyrano[2,3-c]pyrazol-4-yl]-5-(hydroxymethyl)phenyl)ethynyl]azetidine-1-carboxylate. Into a 40-mL vial was placed 6-amino-4-[3-bromo-5-(hydroxymethyl)phenyl]-4-isopropyl-3-methyl-1H-pyrano[2,3-c]pyrazole-5-carbonitrile (4.00 g, 9.919 mmol, 1.00 equiv), DMF (20.00 mL), tert-butyl 3-ethynylazetidine-1-carboxylate (2.16 g, 11.902 mmol, 1.20 equiv), Pd(PPh₃)₄ (1.15 g, 0.992 mmol, 0.10 equiv), CuI (0.38 g, 1.984 mmol, 0.20 equiv) and triethylamine (TEA) (3.01 g, 29.756 mmol, 3.00 equiv). The resulting solution was stirred for 1.5 hr at 80 degrees C. This reaction was repeated 15 times. The reaction mixture was cooled to room temperature. The solids were filtered out. The residue was applied onto a silica gel column with ethyl acetate. This resulted in 20 g (26.67%) of tert-butyl 3-[2-(3-[6-amino-5-cyano-4-isopropyl-3-methyl-1H-pyrano[2,3-c]pyrazol-4-yl]-5-(hydroxymethyl)phenyl)ethynyl]azetidine-1-carboxylate as an off-white solid. LC-MS-KDG-0271-0: (ES, m/z): 504 [M+H]⁺. ¹H-NMR-KDG-0271-0: (300 MHz, DMSO-d₆, ppm): δ 12.12 (s, 1H), 7.28-7.21 (m, 3H), 6.83 (s, 2H), 5.24 (t, J=6.0 Hz, 1H), 4.47-4.45 (m, 2H), 4.17-4.11 (m, 2H), 3.86-3.81 (m, 2H), 3.68-3.63 (m, 1H), 2.74-2.69 (m, 1H), 1.69 (s, 3H), 1.38 (s, 9H), 0.89 (d, J=6.6 Hz, 3H), 0.76 (d, J=6.0 Hz, 3H).

KDG-0320 was synthesized using the synthetic route depicted in Scheme 2.

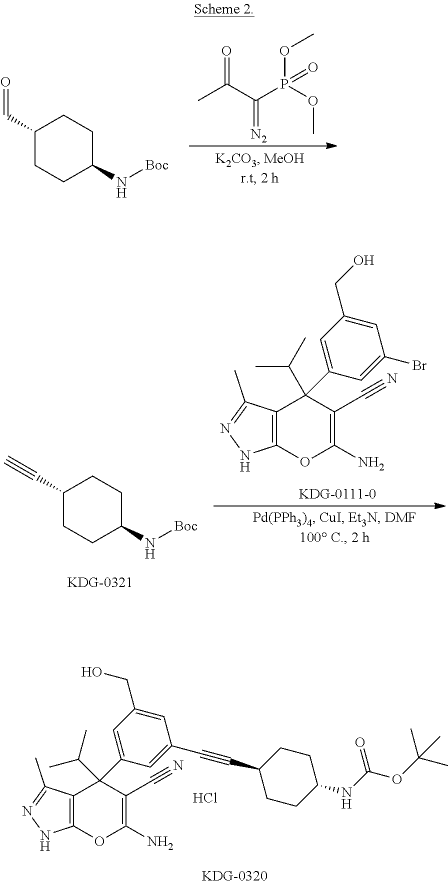

Synthesis of tert-butyl N-[(1r,4r)-4-ethynylcyclohexyl]carbamate. Into a 50-mL, round-bottomed flask was placed tert-butyl N-[(1r,4r)-4-formylcyclohexyl]carbamate (227.00 mg, 0.999 mmol, 1.00 equiv) in MeOH (2.00 mL). K₂CO₃ (275.59 mg, 1.994 mmol, 2.00 equiv) was added. This was followed by the addition of 1-diazo-2-oxopropyl(methoxy)phosphinic acid (287.78 mg, 1.498 mmol, 1.50 equiv) dropwise with stirring at 0° C. The resulting solution was stirred for 2 hr at room temperature. The reaction was then quenched by the addition of 5 mL of water. The resulting solution was extracted with 3×10 mL of ethyl acetate and the organic layers were combined and dried over anhydrous sodium sulfate and concentrated. The residue was applied onto a silica gel column and eluted with ethyl acetate/petroleum ether (1:3). This resulted in 190 mg (85.20%) of tert-butyl N-[(1r,4r)-4-ethynylcyclohexyl]carbamate as colorless oil.

Synthesis of tert-butyl N-[(1r,4r)-4-[2-(3-[6-amino-5-cyano-4-isopropyl-3-methyl-1H-pyrano[2,3-c]pyrazol-4-yl]-5-(hydroxymethyl)phenyl)ethynyl]cyclohexyl]carbamate hydrochloride. Into a 8-mL vial purged and maintained with an inert atmosphere of nitrogen was placed 6-amino-4-[3-bromo-5-(hydroxymethyl)phenyl]-4-isopropyl-3-methyl-1H-pyrano[2,3-c]pyrazole-5-carbonitrile (50.00 mg, 0.124 mmol, 1.00 equiv) in DMF (1.00 mL), tert-butyl N-[(1r,4r)-4-ethynylcyclohexyl]carbamate (33.22 mg, 0.149 mmol, 1.20 equiv), Pd(PPh₃)₄ (14.33 mg, 0.012 mmol, 0.10 equiv), CuI (4.72 mg, 0.025 mmol, 0.20 equiv) and Et₃N (37.64 mg, 0.372 mmol, 3.00 equiv). The resulting solution was stirred for 2 hr at 100° C. The resulting mixture was concentrated. The residue was applied onto a silica gel column and eluted with ethyl acetate/petroleum ether (1:0). The crude product was purified by Preparative HPLC using the following conditions (Prep_HPLC_X03): SunFire Prep C18 OBD 19*150 mm, 5 um, 10 nm, mobile phase, Water (0.05% HCl) and ACN (38% PhaseB up to 52% in 7 min); Detector, UV 220 nm. This resulted in 5 mg (6.93%) of tert-butyl N-[(1r,4r)-4-[2-(3-[6-amino-5-cyano-4-isopropyl-3-methyl-1H-pyrano[2,3-c]pyrazol-4-yl]-5-(hydroxymethyl)phenyl)ethynyl]cyclohexyl]carbamate hydrochloride as a white solid. LC-MS-KDG-0320-0: 544 [M−H—HCl]+. H-NMR-KDG-0320-0: ¹H NMR (300 MHz, DMSO-d₆) δ 7.25 (s, 1H), 7.15 (s, 1H), 7.10 (s, 1H), 4.44 (s, 2H), 3.20-3.16 (m, 1H), 2.74-2.65 (m, 1H), 2.43-2.36 (m, 1H), 1.96 (d, J=10.2 Hz, 2H), 1.77 (d, J=9.9 Hz, 2H), 1.68 (s, 3H), 1.52-1.29 (m, 11H), 1.27-1.08 (m, 2H), 0.89 (d, J=6.6 Hz, 3H), 0.75 (d, J=6.6 Hz, 3H).

KDG-0385-0 and KDG-0388-0 were synthesized using the synthetic route depicted in Scheme 3.

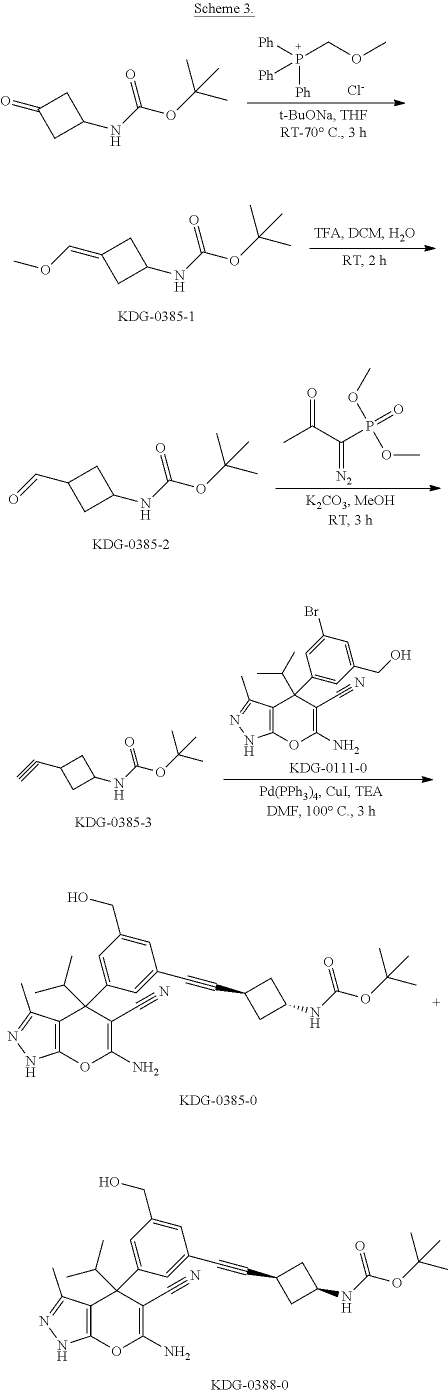

Synthesis of tert-butyl N-[3-(methoxymethylidene) cyclobutyl] carbamate. Into a 250-mL, 3-necked, round-bottomed flask was placed (methoxymethyl)triphenylphosphonium chloride (11.00 g, 31.977 mmol, 1.97 equiv), THE (50.00 mL) and t-BuONa (16 mL, 2.00 equiv). The resulting solution was stirred for 1 hr at room temperature. This was followed by the addition of tert-butyl N-(3-oxocyclobutyl)carbamate (3 g, 16.197 mmol, 1.00 equiv) with stirring at room temperature. The resulting solution was stirred for 2 h at 70° C. in an oil bath. The reaction was then quenched by the addition of 60 mL of water. The resulting solution was extracted with 2×60 mL of ethyl acetate. The resulting mixture was washed with 2×60 mL of brine. The mixture was dried over anhydrous sodium sulfate and concentrated under vacuum. The residue was applied onto a silica gel column with ethyl acetate/petroleum ether (1:5). This resulted in 1.5 g (43.42%) of tert-butyl N-[3-(methoxymethylidene) cyclobutyl] carbamate as a light yellow oil.

Synthesis of tert-butyl N-(3-formylcyclobutyl)carbamate. Into a 100-mL, round-bottomed flask was placed tert-butyl N-[3-(methoxymethylidene)cyclobutyl]carbamate (1.50 g, 7.033 mmol, 1.00 equiv), DCM (30.00 mL), H₂O (2.20 mL) and trifluoroacetic acid (TFA) (1.60 g, 14.032 mmol, 2.00 equiv). The resulting solution was stirred for 2 h at room temperature. The resulting solution was diluted with 60 mL of H₂O. The resulting solution was extracted with 2×60 mL of dichloromethane. The resulting mixture was washed with 2×60 mL of brine. The mixture was dried over anhydrous sodium sulfate and concentrated under vacuum. The residue was applied onto a silica gel column with ethyl acetate/petroleum ether (1:4). This resulted in 1.3 g (92.77%) of tert-butyl N-(3-formylcyclobutyl)carbamate as light yellow oil.

Synthesis of tert-butyl N-(3-ethynylcyclobutyl)carbamate. Into a 100-mL, 3-necked, round-bottomed flask was placed tert-butyl N-(3-formylcyclobutyl)carbamate (1.30 g, 6.524 mmol, 1.00 equiv), MeOH (30.00 mL) and K₂CO₃ (1.80 g, 13.049 mmol, 2.0 equiv). The resulting solution was stirred for 10 minutes (min) at room temperature. This was followed by the addition of Seyferth-Gilbert homologation reagent (1.88 g, 9.787 mmol, 1.5 equiv) dropwise with stirring at 0° C. The resulting solution was stirred for 3 h at room temperature. The solids were filtered out. The resulting mixture was concentrated under vacuum. The residue was applied onto a silica gel column with ethyl acetate/petroleum ether (1:4). This resulted in 900 mg (70.64%) of tert-butyl N-(3-ethynylcyclobutyl)carbamate as an off-white solid.

Synthesis of tert-butyl N-[(1r,3r)-3-[2-(3-[6-amino-5-cyano-4-isopropyl-3-methyl-1H-pyrano[2,3-c]pyrazol-4-yl]-5-(hydroxymethyl)phenyl)ethynyl]cyclobutyl]carbamate & tert-butyl N-[(1s,3s)-3-[2-(3-[6-amino-5-cyano-4-isopropyl-3-methyl-1H-pyrano[2,3-c]pyrazol-4-yl]-5-(hydroxymethyl)phenyl)ethynyl]cyclobutyl]carbamate. Into a 8-mL, sealed tube purged and maintained with an inert atmosphere of nitrogen was placed 6-amino-4-[3-bromo-5-(hydroxymethyl)phenyl]-4-isopropyl-3-methyl-1H-pyrano[2,3-c]pyrazole-5-carbonitrile (100.00 mg, 0.248 mmol, 1.00 equiv), tert-butyl N-(3-ethynylcyclobutyl)carbamate (96.84 mg, 0.496 mmol, 2.00 equiv), DMF (2.00 mL), CuI (9.45 mg, 0.050 mmol, 0.2 equiv), TEA (62.73 mg, 0.620 mmol, 2.5 equiv) and Pd(PPh₃)₄ (28.65 mg, 0.025 mmol, 0.1 equiv). The resulting solution was stirred for 2 h at 100° C. in an oil bath. The solids were filtered out. The crude product was purified by preparative HPLC using the following conditions: Column, XBridge C18 OBD; mobile phase, A: water (0.05% NH₃·H₂O) B: ACN; Gradient: 36-50% B in 7 min; Flow rate: 20 ml/min; Detector, 254 nm. This resulted in 3 mg (2.34%) of tert-butyl N-[(1r,3r)-3-[2-(3-[6-amino-5-cyano-4-isopropyl-3-methyl-1H-pyrano[2,3-c]pyrazol-4-yl]-5-(hydroxymethyl)phenyl)ethynyl]cyclobutyl]carbamate as an off-white solid. This resulted in 6 mg (4.67%) of tert-butyl N-[(1s,3s)-3-[2-(3-[6-amino-5-cyano-4-isopropyl-3-methyl-1H-pyrano[2,3-c]pyrazol-4-yl]-5-(hydroxymethyl)phenyl)ethynyl]cyclobutyl]carbamate as an off-white solid.

LC-MS-KDG-0385-0: (ES, m/z): 518 [M+H]+. ¹H-NMR-KDG-0385-0: (300 MHz, DMSO-d₆, ppm): δ 12.11 (s, 1H), 7.27 (d, J=11.2 Hz, 2H), 7.18 (d, J=11.8 Hz, 2H), 6.82 (s, 2H), 5.23 (t, J=5.8 Hz, 1H), 4.46 (d, J=5.7 Hz, 2H), 4.20 (d, J=8.0 Hz, 1H), 3.16 (s, 1H), 2.71 (d, J=6.8 Hz, 1H), 2.31-2.24 (m, 4H) 1.70 (s, 3H), 1.38 (s, 9H), 0.90 (d, J=6.5 Hz, 3H), 0.77 (d, J=6.4 Hz, 3H).

LC-MS-KDG-0388-0: (ES, m/z): 518 [M+H]+. ¹H-NMR-KDG-0388-0: (300 MHz, DMSO-d₆, ppm): δ 12.11 (s, 1H), 7.25 (s, 1H), 7.22-7.09 (m, 3H), 6.83 (s, 2H), 5.23 (t, J=5.8 Hz, 1H), 4.46 (d, J=5.7 Hz, 2H), 3.87 (d, J=8.6 Hz, 1H), 2.87-2.80 (m, 1H), 2.73-2.69 (m, 1H), 2.58-2.55 (m, 2H), 2.09-1.99 (m, 2H), 1.69 (s, 3H), 1.37 (s, 9H), 0.90 (d, J=6.5 Hz, 3H), 0.76 (d, J=6.4 Hz, 3H).

KDG-0388-0 was separated into its individual enantiomers, depicted in Scheme 4.

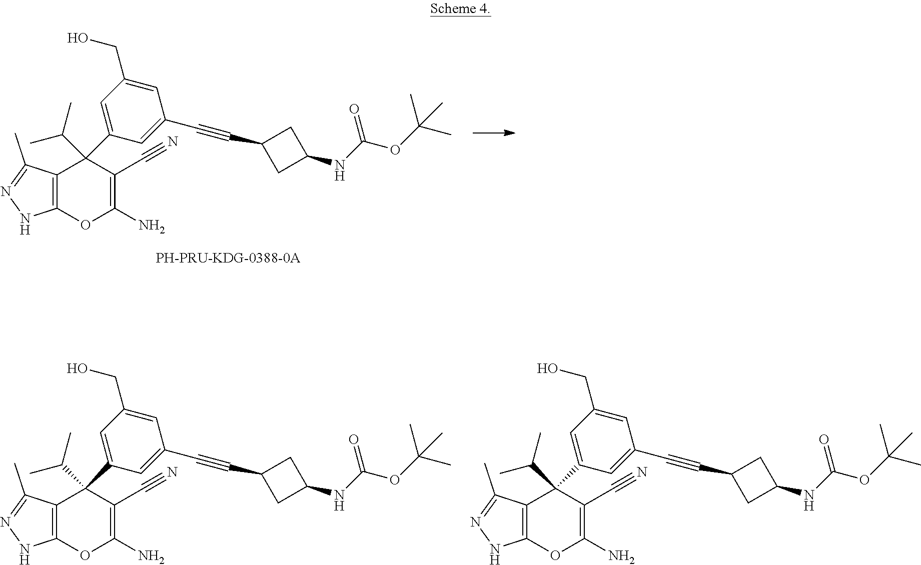

Chiral Separation of Enantiomers of KDG-0388-0. 80 Milligrams of tert-butyl N-[(1s,3s)-3-[2-(3-[6-amino-5-cyano-4-isopropyl-3-methyl-1H-pyrano[2,3-c]pyrazol-4-yl]-5-(hydroxymethyl)phenyl)ethynyl]cyclobutyl]carbamate obtained in accordance with the synthetic route depicted in Scheme 3 was purified by chiral HPLC using the following separation conditions: Column: CHIRALPAK IG, 3*25 cm, Sum; Mobile Phase: A: Hex:DCM=3:1, B: EtOH; Flow rate: 30 mL/min; Gradient: 10% B to 10% B in 10 min; detector: 220/254 nm; P1: 7 min; P2: 8 min; Injection Volumn: 0.3 mL; Number of Runs: 20. 29 Milligrams of tert-butyl N-[(1s,3s)-3-(2-[3-[(4S)-6-amino-5-cyano-4-isopropyl-3-methyl-1H-pyrano[2,3-c]pyrazol-4-yl]-5-(hydroxymethyl)phenyl]ethynyl)cyclobutyl]carbamate (KDG-0388-P1, RT=2.02 min in ANA-Chiral-HPLC IG-3 column) and 30 mg of tert-butyl N-[(1s,3s)-3-(2-[3-[(4R)-6-amino-5-cyano-4-isopropyl-3-methyl-1H-pyrano[2,3-c]pyrazol-4-yl]-5-(hydroxymethyl)phenyl]ethynyl)cyclobutyl]carbamate (KDG-0388-P2, RT=2.53 min in ANA-Chiral-HPLC IG-3 column) were obtained.

LC-MS-KDG-0388-P1: (ES, m/z): 516 [M−H]⁻. ¹H-NMR-KDG-0388-P1: (300 MHz, DMSO-d₆, ppm): δ 12.11 (s, 1H), 7.25 (s, 1H), 7.16-7.13 (m, 3H), 6.83 (s, 2H), 5.22 (t, J=6.0 Hz, 1H), 4.45 (d, J=5.4 Hz, 2H), 3.98-3.79 (m, 1H), 2.95-2.78 (m, 1H), 2.75-2.64 (m, 1H), 2.60-2.55 (m, 2H), 2.12-1.95 (m, 2H), 1.69 (s, 3H), 1.37 (s, 9H), 0.89 (d, J=6.6 Hz, 3H), 0.76 (d, J=6.3 Hz, 3H).

LC-MS-KDG-0388-P2: (ES, m/z): 516 [M−H]⁻. ¹H-NMR-KDG-0388-P2: (300 MHz, DMSO-d₆, ppm): δ 12.11 (s, 1H), 7.25 (s, 1H), 7.16-7.13 (m, 3H), 6.83 (s, 2H), 5.22 (t, J=6.0 Hz, 1H), 4.45 (d, J=5.4 Hz, 2H), 3.98-3.79 (m, 1H), 2.95-2.78 (m, 1H), 2.75-2.64 (m, 1H), 2.60-2.55 (m, 2H), 2.12-1.95 (m, 2H), 1.69 (s, 3H), 1.37 (s, 9H), 0.89 (d, J=6.6 Hz, 3H), 0.76 (d, J=6.3 Hz, 3H).

KDG-0502 was synthesized using the synthetic route depicted in Scheme 5.

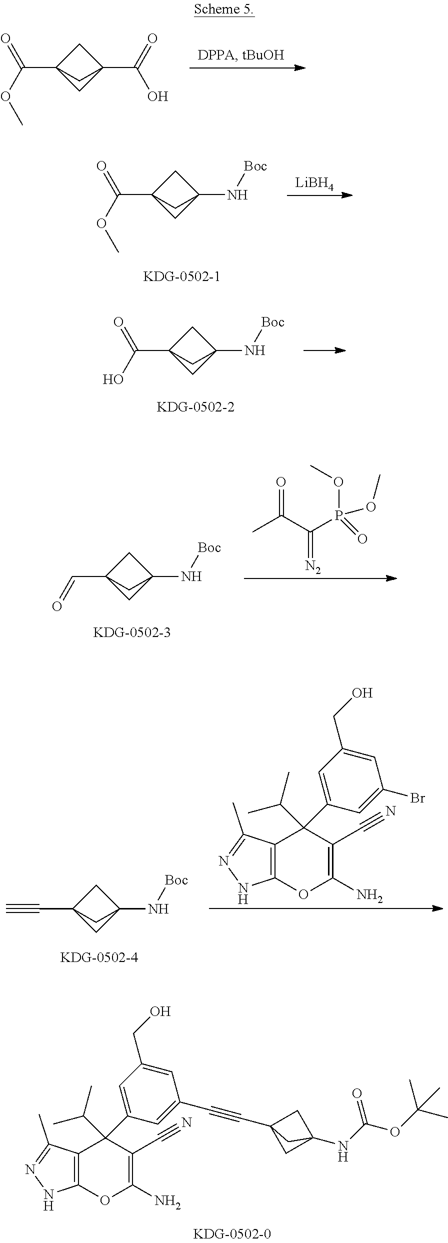

Synthesis of methyl 3-[(tert-butoxycarbonyl)amino]bicyclo[1.1.1]pentane-1-carboxylate. Into a 100-mL, round-bottomed flask was placed t-BuOH (4.00 mL), toluene (20.00 mL), 3-(methoxycarbonyl)bicyclo[1.1.1]pentane-1-carboxylic acid (1000.00 mg, 5.877 mmol, 1.00 equiv), TEA (594.66 mg, 5.877 mmol, 1 equiv) and DPPA (1617.26 mg, 5.877 mmol, 1.00 equiv). The resulting solution was stirred for 4 hr at 25° C. under N₂ atmosphere. The resulting solution was allowed to react, with stirring, for an additional 16 hr at 80° C. The reaction mixture was cooled to 25° C. with a water bath. The reaction was then quenched by the addition of 50 mL of water. The resulting solution was extracted with 3×30 mL of ethyl acetate and the organic layers were combined and dried over anhydrous sodium sulfate and concentrated. The residue was applied onto a silica gel column and eluted with ethyl acetate/petroleum ether (1:1). The collected fractions were combined and concentrated. This resulted in 860 mg (54.59%) of methyl 3-[(tert-butoxycarbonyl)amino]bicyclo[1.1.1]pentane-1-carboxylate as a white solid.

Synthesis of tert-butyl N-[3-(hydroxymethyl)bicyclo[1.1.1]pentan-1-yl]carbamate. Into a 100-mL, 3-necked, round-bottomed flask was placed TUE (20.00 mL) and methyl 3-[(tert-butoxycarbonyl)amino]bicyclo[1.1.1]pentane-1-carboxylate (860.00 mg, 3.564 mmol, 1.00 equiv). This was followed by the addition of LiBH₄ (310.57 mg, 14.257 mmol, 4 equiv) in several batches at 0° C. The resulting solution was stirred for 16 hr at 25° C. The reaction was then quenched by the addition of 50 mL of water. The resulting solution was extracted with 3×30 mL of ethyl acetate and the organic layers were combined and dried over anhydrous sodium sulfate and concentrated. The residue was applied onto a silica gel column and eluted with ethyl acetate/petroleum ether (1:1). The collected fractions were combined and concentrated. This resulted in 700 mg (82.88%) of tert-butyl N-[3-(hydroxymethyl)bicyclo[1.1.1]pentan-1-yl]carbamate as a white solid.

Synthesis of tert-butyl N-[3-formylbicyclo[1.1.1]pentan-1-yl]carbamate. Into a 50-mL, round-bottomed flask was placed DCM (10.00 mL), tert-butyl N-[3-(hydroxymethyl)bicyclo[1.1.1]pentan-1-yl]carbamate (300.00 mg, 1.407 mmol, 1.00 equiv). This was followed by the addition of Dess-Martin Reagent (715.93 mg, 1.688 mmol, 1.20 equiv) in several batches. The resulting solution was stirred for 16 hr at 25° C. The reaction was then quenched by the addition of 50 mL of water. The resulting solution was extracted with 3×30 mL of dichloromethane and the organic layers were combined and dried over anhydrous sodium sulfate and concentrated. The residue was applied onto a silica gel column and eluted with ethyl acetate/petroleum ether (1:1). The collected fractions were combined and concentrated. This resulted in 140 mg (42.40%) of tert-butyl N-[3-formylbicyclo[1.1.1]pentan-1-yl]carbamate as a white solid.

Synthesis of tert-butyl N-[3-ethynylbicyclo[1.1.1]pentan-1-yl]carbamate. Into a 50-mL, round-bottomed flask was placed MeOH (5.00 mL), tert-butyl N-[3-formylbicyclo[1.1.1]pentan-1-yl]carbamate (140.00 mg, 0.663 mmol, 1.00 equiv), K₂CO₃ (274.76 mg, 1.988 mmol, 3 equiv) and Seyferth-Gilbert homologation reagent (190.96 mg, 0.994 mmol, 1.50 equiv). The resulting solution was stirred for 2 hr at 25° C. The reaction was then quenched by the addition of 30 mL of water. The resulting solution was extracted with 3×20 mL of ethyl acetate and the organic layers were combined and dried over anhydrous sodium sulfate and concentrated. The residue was applied onto a silica gel column with ethyl acetate/petroleum ether (1:1). The collected fractions were combined and concentrated. This resulted in 110 mg (72.07%) of tert-butyl N-[3-ethynylbicyclo[1.1.1]pentan-1-yl]carbamate as a white solid.

Synthesis of tert-butyl N-[3-[2-(3-[6-amino-5-cyano-4-isopropyl-3-methyl-1H-pyrano[2,3-c]pyrazol-4-yl]-5-(hydroxymethyl)phenyl)ethynyl]bicyclo[1.1.1]pentan-1-yl]carbamate. Into a 40-mL vial was placed DMF (2.00 mL), tert-butyl N-[3-ethynylbicyclo[1.1.1]pentan-1-yl]carbamate (50.00 mg, 0.241 mmol, 1.00 equiv), 6-amino-4-[3-bromo-5-(hydroxymethyl)phenyl]-4-isopropyl-3-methyl-1H-pyrano[2,3-c]pyrazole-5-carbonitrile (77.83 mg, 0.193 mmol, 0.80 equiv), Pd(PPh₃)₄ (27.88 mg, 0.024 mmol, 0.1 equiv), CuI (9.19 mg, 0.048 mmol, 0.2 equiv) and TEA (73.23 mg, 0.724 mmol, 3 equiv). The resulting solution was stirred for 2 h at 100° C. under N₂ atmosphere. The reaction mixture was cooled to 25° C. with a water bath. The reaction was then quenched by the addition of 20 mL of water. The resulting solution was extracted with 3×15 mL of ethyl acetate and the organic layers were combined and dried over anhydrous sodium sulfate and concentrated. The residue was applied onto a silica gel column and eluted with ethyl acetate/petroleum ether (1:1). The collected fractions were combined and concentrated. The residue was dissolved in 2 mL of MeOH. The crude product was purified by preparative HPLC using the following conditions (Prep-HPLC-006): Column, XBridge Shield RP18 OBD Column, 5 um, 19*150 mm; mobile phase, Water (0.1% NH₃·H₂O) and ACN (36.0% ACN up to 49.0% in 6 min, hold 95.0% in 1 min, hold 36.0% in 1 min); Detector, UV 220 nm. This resulted in 6 mg (4.60%) of tert-butyl N-[3-[2-(3-[6-amino-5-cyano-4-isopropyl-3-methyl-1H-pyrano[2,3-c]pyrazol-4-yl]-5-(hydroxymethyl)phenyl)ethynyl]bicyclo[1.1.1]pentan-1-yl]carbamate as a white solid. LC-MS-KDG-0502-0: (ES, m/z) 530 [M+H]+. H-NMR-KDG-0502-0: ¹H NMR (300 MHz, DMSO-d6): δ 12.12 (s, 1H), 7.63 (bs, 1H), 7.28 (s, 1H), 7.19 (s, 1), 7.14 (s, 1H), 6.84 (s, 2H), 5.23 (m, 1H), 4.47-4.45 (m, 2H), 2.76-2.72 (m, 1H), 2.23 (s, 6H), 1.68 (s, 3H), 1.38 (s, 9H), 0.91-0.88 (m, 3H), 0.77-0.75 (m, 3H).

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A compound of the following structural formula: 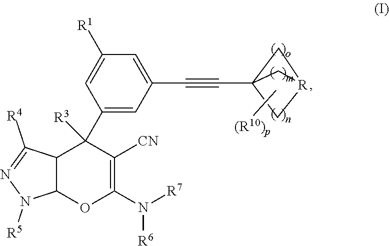
or a pharmaceutically acceptable salt thereof, wherein:
m is 1 or 2;
n is 0, 1 or 2;
is 0, 1, 2 or 3, wherein when o is 0, the ring containing R is monocyclic;
p is 0, 1, 2, 3, 4 or 5;
R is —C(H)(N(R⁸)C(O)R²)—, —C(CH₃)(N(R⁸)C(O)R²)—, —C(H)(N(R⁸)CH₂C(O)R²)—, —C(H)(C(O)R²)—, —N(C(O)R²)—, —C(H)(CH₂N(R⁸)C(O)R²)—, —C(H)(CH₂OC(O)R²)—, —N(CH₂C(O)R²)—, —C(H)(hydroxy(C₁-C₁₀)alkyl)-, —C(CH₃)(OH)—, —N(hydroxy(C₁-C₁₀)alkyl)-, —C(CH₃)(OC(O)(C₁-C₁₀)alkyl)-, —C(O)—, —N(H)—, —O—, —C(H)((C₅-C₆)heteroaryl)-, —C(H)(N(R⁸)(C₅-C₆)heteroaryl)-, —C(H)((C₃-C₇)heterocyclyl)- or —N((C₅-C₆)heteroaryl)- when o is 0, and —C(N(R⁸)C(O)R²)—, —CCH₂(N(R⁸)C(O)R²)—, —C(N(R⁸)CH₂C(O)R²)—, —C(C(O)NR^{a}R^{b})—, —C(hydroxy(C₁-C₁₀)alkyl)-, —C((C₅-C₆)heteroaryl)-, —C(N(R⁸)(C₅-C₆)heteroaryl)- or —C((C₃-C₇)heterocyclyl)- when o is other than 0, wherein the heteroaryl can be substituted with one or more R¹¹, and the heterocyclyl can be substituted with one or more substituents selected from oxo or R¹¹;
R¹ is (C₁-C₆)alkyl, hydroxy(C₁-C₆)alkyl, halo(C₁-C₆)alkyl, (C₃-C₆)cycloalkyl, halo or cyano;
R² is (C₁-C₁₀)alkoxy, (C₁-C₁₀)alkoxy(C₁-C₁₀)alkoxy, hydroxy(C₁-C₁₀)alkoxy, halo(C₁-C₁₀)alkoxy, (C₃-C₆)cycloalkoxy, (C₆-C₁₂)aryl(C₁-C₁₀)alkoxy, (C₅-C₁₂)heteroaryl(C₁-C₁₀)alkoxy, (C₁-C₁₀)alkyl, —NR^{a}R^{b} or —CH₂NR^{a}R^{b}, wherein the cycloalkoxy can be substituted with one or more R¹²;
R^{a} is hydrogen or (C₁-C₁₀)alkyl;
R^{b} is (C₁-C₁₀)alkyl;
R³ is (C₁-C₆)alkyl or (C₁-C₆)cycloalkyl;
R⁴ is (C₁-C₆)alkyl, (C₁-C₆)haloalkyl, hydroxy(C₁-C₆)alkyl or (C₃-C₆)cycloalkyl;
R⁵, R⁶ and R⁷ are each hydrogen;
each R⁸ is independently hydrogen or (C₁-C₁₀)alkyl; and
R¹⁰, R¹¹ and R¹², for each occurrence, are independently halo, hydroxy, cyano, (C₁-C₆)alkyl, halo(C₁-C₆)alkyl, hydroxy(C₁-C₆)alkyl.

2. The compound of claim 1, having the following structural formula: 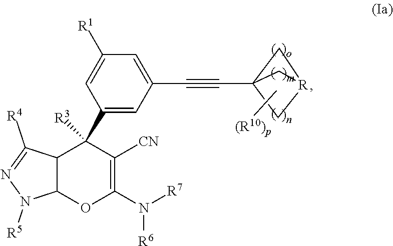
or a pharmaceutically acceptable salt thereof.

3. The compound of claim 1, having the following structural formula: 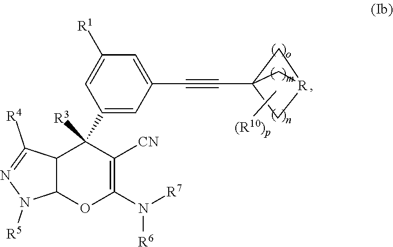
or a pharmaceutically acceptable salt thereof.

4. The compound of any one of claims 1-3, wherein m is 1.

5. The compound of any one of claims 1-3, wherein m is 2.

6. The compound of any one of claims 1-5, wherein n is 1 or 2.

7. The compound of any one of claims 1-6, wherein o is 0 or 1.

8. The compound of any one of claims 1-3, wherein m is 1, n is 1 and o is 0.

9. The compound of any one of claims 1-3, wherein m is 2, n is 2 and o is 0.

10. The compound of any one of claims 1-3, wherein m is 1, n is 1 and o is 1.

11. The compound of any one of claims 1-10, wherein p is 0 or 1.

12. The compound of claim 11, wherein p is 0.

13. The compound of any one of claims 1-12, wherein R is —C(H)(N(R⁸)C(O)R²)—, —C(CH₃)(N(R⁸)C(O)R²)— or —N(C(O)R²)— when o is 0, and —C(N(R⁸)C(O)R²)— when o is other than 0.

14. The compound of any one of claims 1-12, wherein R is —C(H)((C₅-C₆)heteroaryl)-, —C(H)(N(R⁸)(C₅-C₆)heteroaryl)- or —N((C₅-C₆)heteroaryl)- when o is 0, and —C((C₅-C₆)heteroaryl)- or —C(N(R⁸)(C₅-C₆)heteroaryl)- when o is other than 0, wherein the heteroaryl can be substituted with one or more R¹¹.

15. The compound of one of claims 1-14, wherein R¹ is —CH₂OH, —C(CH₃)₂OH, —CH₂F, —CF₃ or —F.

16. The compound of claim 15, wherein R¹ is —CH₂OH.

17. The compound of any one of claims 1-16, wherein R² is (C₁-C₁₀)alkoxy.

18. The compound of claim 17, wherein R² is tert-butoxy.

19. The compound of any one of claims 1-16, wherein R^{a} is hydrogen.

20. The compound of any one of claims 1-16 and 19, wherein R^{b} is tert-butyl.

21. The compound of any one of claims 1-20, wherein R³ is isopropyl, n-propyl, ethyl, methyl, cyclopropyl or cyclobutyl.

22. The compound of claim 21, wherein R³ is isopropyl.

23. The compound of any one of claims 1-22, wherein R⁴ is methyl.

24. The compound of any one of claims 1-23, wherein R⁸ is hydrogen.

25. The compound of any one of claims 1-24, wherein R¹⁰, for each occurrence, is independently halo, (C₁-C₆)alkyl or halo(C₁-C₆)alkyl.

26. The compound of claim 25, wherein R¹⁰ for each occurrence is fluoro, methyl or trifluoromethyl.

27. The compound of any one of claims 1-12 and 14-26, wherein R¹¹, for each occurrence, is independently (C₁-C₆)alkyl.

28. The compound of claim 27, wherein R¹¹, for each occurrence, is independently methyl or isopropyl.

29. The compound of any one of claims 1-13 and 15-28, having the following structural formula: 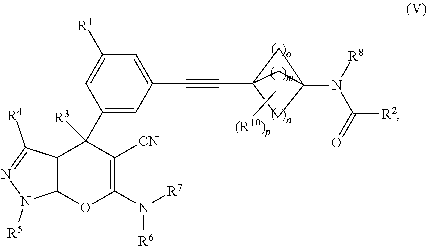
or a pharmaceutically acceptable salt thereof.

30. The compound of claim 29, having the following structural formula. 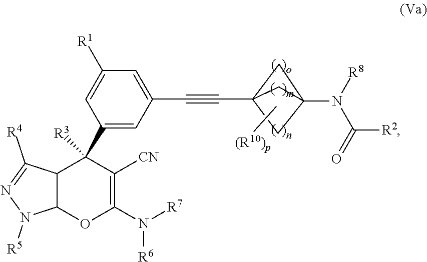
or a pharmaceutically acceptable salt thereof.

31. The compound of claim 29, having the following structural formula: 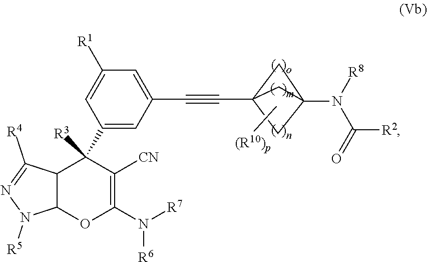
or a pharmaceutically acceptable salt thereof.

32. The compound of any one of claims 1-13 and 15-28, having the following structural formula: 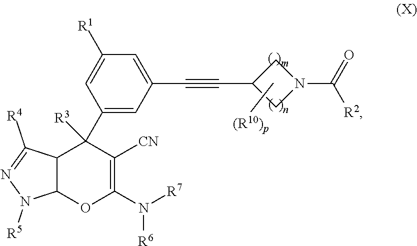
or a pharmaceutically acceptable salt thereof.

33. The compound of claim 32, having the following structural formula: 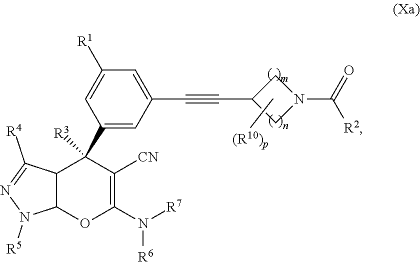
or a pharmaceutically acceptable salt thereof.

34. The compound of claim 32, having the following structural formula: 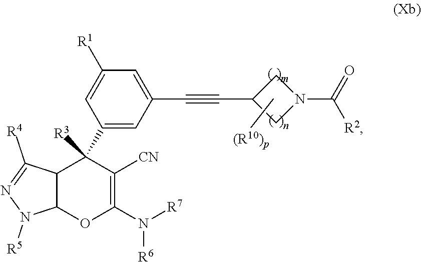
or a pharmaceutically acceptable salt thereof.

35. The compound of claim 1, having the following structural formula: 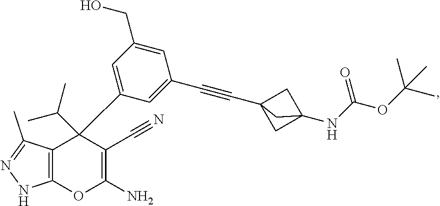 [SMILES: Cc1n[nH]c2c1C(c1cc(C#CC34CC(NC(=O)OC(C)(C)C)(C3)C4)cc(CO)c1)(C(C)C)C(C#N)=C(N)O2]
or a pharmaceutically acceptable salt thereof.

36. The compound of claim 1, having the following structural formula: 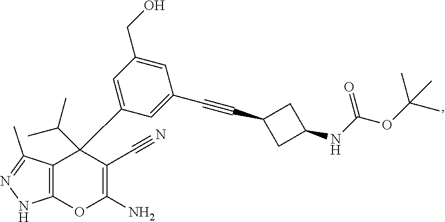 [SMILES: Cc1n[nH]c2c1C(c1cc(C#C[C@H]3C[C@@H](NC(=O)OC(C)(C)C)C3)cc(CO)c1)(C(C)C)C(C#N)=C(N)O2]
or a pharmaceutically acceptable salt thereof.

37. The compound of claim 1, having the following structural formula: 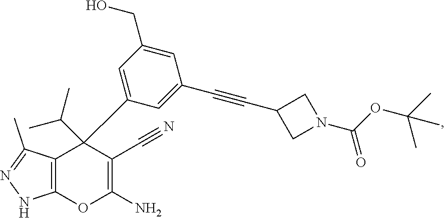 [SMILES: Cc1n[nH]c2c1C(c1cc(C#CC3CN(C(=O)OC(C)(C)C)C3)cc(CO)c1)(C(C)C)C(C#N)=C(N)O2]
or a pharmaceutically acceptable salt thereof.

38. The compound of claim 1, having the following structural formula: 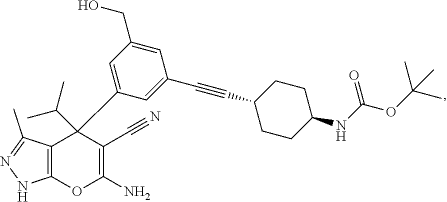 [SMILES: Cc1n[nH]c2c1C(c1cc(C#C[C@H]3CC[C@H](NC(=O)OC(C)(C)C)CC3)cc(CO)c1)(C(C)C)C(C#N)=C(N)O2]
or a pharmaceutically acceptable salt thereof.

39. A compound of any one of the structural formulas of Table 1, or a pharmaceutically acceptable salt thereof.

40. A composition, comprising a compound of any one of claims 1-39, or a pharmaceutically acceptable salt thereof, and a pharmaceutically acceptable carrier.

41. A method of treating a disease, disorder or condition associated with serine hydroxymethyl transferase (SHMT) activity or expression in a subject in need thereof, comprising administering to the subject a therapeutically effective amount of a compound of any one of claims 1-39, or a pharmaceutically acceptable salt thereof.

42. A method of treating a cancer in a subject in need thereof, comprising administering to the subject a therapeutically effective amount of a compound of any one of claims 1-39, or a pharmaceutically acceptable salt thereof.

43. The method of claim 42, wherein the cancer comprises a solid tumor.

44. The method of claim 42, wherein the cancer is a hematologic cancer.

45. A method of treating an autoimmune disease in a subject in need thereof, comprising administering to the subject a therapeutically effective amount of a compound of any one of claims 1-39, or a pharmaceutically acceptable salt thereof.

46. The method of claim 45, wherein the autoimmune disease is rheumatoid arthritis, multiple sclerosis, systemic lupus erythematosus, type 1 diabetes mellitus, Guillain-Barre syndrome, chronic inflammatory demyelinating polyneuropathy, Graves' disease, Hashimoto's thyroiditis, myasthenia gravis, inflammatory bowel disease, Crohn's disease, polymyositis, dermatomyositis, inflammatory myositis, ankylosing spondolytis, ulcerative colitis, psoriasis, vasculitis, sarcoidosis, eczema, vasculitis, Sjogren's disease or transplant rejection.

47. A method of treating fibrosis or a fibrotic disease in a subject in need thereof, comprising administering to the subject a therapeutically effective amount of a compound of any one of claims 1-39, or a pharmaceutically acceptable salt thereof.

48. The method of claim 47, wherein the fibrosis or fibrotic disease is systemic sclerosis, scleroderma, pulmonary fibrosis, idiopathic pulmonary fibrosis, primary biliary cholangitis, liver fibrosis, cirrhosis, fatty liver disease, non-alcoholic fatty liver disease, chronic hepatitis B or C, heart fibrosis, post-myocardial infarction cardiac changes, interstitial fibrosis, replacement fibrosis, mediastinal fibrosis, bone marrow fibrosis, myelofibrosis, pancreas fibrosis, skin fibrosis, nephrogenic systemic fibrosis, keloid formation, renal fibrosis, Peyronie's disease, contractures, arthrofibrosis, Crohn's disease, adhesive capsulitis, excessive surgical scarring or retroperitoneal cavity fibrosis.

49. The method of any one of claims 41-48, further comprising administering to the subject an additional therapeutic agent.

50. The method of claim 49, wherein the additional therapeutic agent is a rescue agent.

51. The method of claim 50, wherein the rescue agent is formate, a formate ester, glycine, leucovorin, or a pharmaceutically acceptable salt of any of the foregoing.
